(19) United States
(12) Patent Application Publication
(10) Pub. No.: US 2023/0032082 A1
(43) Pub. Date: Feb. 2, 2023

(54) SPATIAL BARCODING

(71) Applicant: CANCER RESEARCH TECHNOLOGY LIMITED, London (GB)

(72) Inventors: Gregory HANNON, Cambridge (GB); Dario BRESSAN, Cambridge (GB); Shankar BALASUBRAMANIAN, Cambridge (GB); Giorgia BATTISTONI, Cambridge (GB)

(73) Assignee: CANCER RESEARCH TECHNOLOGY LIMITED, London (GB)

(21) Appl. No.: 17/781,650

(22) PCT Filed: Dec. 11, 2020
(86) PCT No.: PCT/GB2020/053202

§ 371 Date: Jun. 1, 2022

(30) Foreign Application Priority Data
Dec. 12, 2019 (GB) 1918340.9

(51) Int. Cl. C12Q 1/6841; C12N 15/10; G01N 33/58; C12Q 1/6806

(52) U.S. Cl. CPC C12Q 1/6841; C12N 15/1065; G01N 33/58; C12Q 1/6806

(57) ABSTRACT

The present invention relates to a method of spatially barcoding a given location on a substrate, and further to spatially barcoding detection probes present in a sample such as a biological tissue specimen for the purposes of analysing molecular features present in the tissue. Such analysis may include: i) the spatial expression of one or more biological molecules, specifically; ii) the spatial analysis of the transcriptome and/or iii) the spatial analysis of the proteome, including post-translational protein modifications. The invention further relates to various component products for performing such methods that include reagents kits, instrumentation and software.

### FIELD OF THE INVENTION

The present invention relates to a method of spatially barcoding a given location, and further to spatially barcoding detection probes present in a sample such as a biological tissue specimen for the purposes of analysing molecular features present in the tissue. Such analysis may include, for example, one or more of the following: i) the spatial expression of one or more biological molecules, specifically; ii) the spatial analysis of the transcriptome and/or iii) the spatial analysis of the proteome, including post-translational protein modifications. The invention further relates to various component products for performing such methods that include reagents kits, instrumentation and software.

### INTRODUCTION

In situ analysis of the expression of biological molecules is an area of technology that has rapidly developed in recent years. In particular, the development of in situ transcriptomics and multiplexed histochemistry analysis techniques, that allow the determination of what genes are being expressed and/or what biological markers may be present, and to what level in any given location of a given tissue sample, have gained increasing popularity, and enabled a whole new range of biological investigations.

Historically, methods allowing the measurement of many biological molecules at once with high-throughput have provided data on average expression levels in a given sample, but without any context of where the molecules are being expressed. More recently, single-cell analysis techniques have been developed, in which cells from disaggregated tissues are analysed individually. While these method provide more detailed information on the biological processes happening in the sample, and allow the identification of rare cell populations contributing to them, the absence of real spatial information is a significant hurdle to research. Since all of biology happens in space, the function of a given biological molecule in a process can only be fully understood by considering the spatial context in which the molecule itself is acting.

The field has started to address the need for spatial information by the provision of various in situ transcriptomics and proteomics methods, involving a means of spatial detection of gene or protein expression. There are also families of methods for spatial profiling of metabolites and other biological molecules. These methods can be broadly considered as two groups; image-based methods and image-free methods.

In image-based methods for gene expression measurement, the RNAs contained into the biological tissue are first contacted by DNA probes of complementary sequence. In some techniques, the probes are directly used for the detection and identification of each RNA molecule through fluorescence, using a variety of detection schemes which in some cases include signal amplification through branched DNA, hybridization chain reaction, or rolling circle amplification. In other techniques, the probes are used as primers for reverse transcription of the RNA, producing a complementary DNA molecule for each RNA transcript which can be amplified and detected through in-situ sequencing. Such methods include merFISH, seqFISH, starMAP, FISSEQ, ISS, and BARISTAseq. In all of these methods, the identification of multiple types of RNA molecules (corresponding to different genes) is achieved by repeated cycles of fluorescence imaging in which individual molecules are detected as fluorescent spots. These methods are limited in their ability to achieve single molecule imaging, since the image signals can have low intensity, be difficult to discriminate and suffer from auto-fluorescence or background noise. Furthermore, these methods do not allow the identification of very abundant RNA molecules, since these produce signals that overlap spatially and can't be decoded (crowding). The need for repeated imaging cycles is also a significant issue for many of these technologies, as the images from each cycle need to be exactly aligned to within a few nanometres of precision, which is technically challenging. Finally, these methods are time consuming, since they require a very high magnification in order to achieve single-molecule resolution, and can only image a very small area of the tissue each time. The time required for a full experiment scales both with the area of the tissue being analysed, and with the number of features (genes) being detected, which limits the amount of information that can be recovered.

Image-free gene expression measurement methods avoid the limitations that result from imaging the tissue sample, and rely on sequencing techniques to determine location of a given RNA molecule. This requires the fusion of a spatial DNA barcode with the molecule to be detected, achieved by using the spatial barcode as primer for reverse transcription, which produces a spatially barcoded cDNA. These methods are much faster as the imaging time, which is relatively slow overall, is removed and data analysis is much more simple. Such methods include; 10× Visium, SlideSeq, and HDST. However, these methods have a lower efficiency, and are commonly limited to capturing no more than 10% of the RNA content of a cell due to limitations in the reverse transcription step. In addition, they all require some sort of solid support on which the spatial DNA barcode is arrayed prior to adding the sample. This support is expensive to produce, fragile, and often results in low spatial resolution, which does not allow capture of information from single cells. Furthermore, the spatial barcoding does not follow the structure of the tissue, but the spatial addresses are arranged either in a regular square grid or randomly. This results in parts of the tissue not being analysed, and in some spatial barcodes overlapping multiple cells, producing imprecise information.

In-situ proteomics methods use antibodies conjugated with probes that can be fluorescent molecules, heavy metal isotopes bound by a chemical polymer, or DNA molecules. The tissue is contacted with a library of antibodies so that multiple biological markers (typically protein or protein modifications) are bound by the antibody and linked to the probes. These methods include CODEX, Imaging mass cytometry, MIBI, 4i, and Miltenyi MACSima. The probes are then detected by mass spectrometry or by fluorescence imaging (in the latter case, through subsequent imaging cycles as described above for the gene expression measurements). These methods suffer from many of the same issues described above for imaging-based gene expression measurements. Furthermore, in-situ proteomics measurements exist mostly as a separate class of techniques, and have not been successfully integrated with gene expression measurements in a high-throughput way allowing measurements of hundreds of genes and proteins together in the same sample. Related antibody free methods can measure a variety of small molecules and potentially peptides and proteins by direct mass spectrometric imaging.

The present invention aims to solve one or more of the above-mentioned problems by the provision of a novel image-free in situ spatial barcoding method that can be used generally for spatially encoding molecular information, and in particular for the spatial analysis of the transcriptome or proteome of a tissue sample, or indeed any molecule or feature that can be recognized by an affinity reagent in a tissue of interest.

The methods of the present invention are based on a technique of encoding spatial barcodes into nucleic acid molecules through the use of light as a tool to guide spatial barcode assembly onto the molecules, so as to allow the identification of the original spatial position of each molecule following high-throughput sequencing.

Advantageously, the methods of the present invention use simple commonly available instruments such as a light microscope and standard tissue slides to provide a method for spatially labelling biological molecules within an area of tissue, down to single cells or sub-cellular compartments, with a resolution equal or below the diffraction limit of UV light, at high efficiency, and without many of the issues related to single-molecule imaging. The method enables the quantification and spatial localization of genes, proteins and other biological markers individually, or at the same time, and in the same sample, using high-throughput sequencing. The method has single-molecule sensitivity, high throughput, and produces data that can be readily analysed using techniques available in the field and further incorporates features allowing the control of some significant sources of error such as off-target probe binding and background noise. The method of the invention is therefore cheaper, quicker, and more powerful (due to the higher sensitivity, the possibility of analysing gene expression ad protein/marker expression at the same time, and the ease of analysis) than existing methods, whilst still extracting detailed spatial information regarding the molecular make-up of a tissue.

### STATEMENTS OF INVENTION

According to a first aspect of the present invention, there is provided a method of spatially barcoding one or more locations of a substrate, comprising:
(a) Binding one or more root nucleic acid molecules to the or each location on which the spatial barcode will be constructed, wherein the or each root molecule may comprise a photocleavable group;
(b) Optionally, if the or each root molecule does not comprise a photocleavable group, adding a photocleavable group to the or each root molecule;
(c) Illuminating a location of interest on the substrate to be spatially barcoded, wherein the illumination cleaves or alters the photocleavable group of the or each root molecule present within the location;
(d) Adding an index sequence to the or each root molecule within the location illuminated in step (b), wherein the index sequence comprises a photocleavable group;
(e) Repeating steps (c) and (d) until the desired index sequences are added to form a spatial barcode attached to the or each root molecule within the location.

Suitably the substrate may be any surface. Suitably the substrate may be an inert substrate such as glass, plastic, etc. Suitably the substrate may be living, suitably the substrate may be a specimen or tissue sample.

According to a second aspect of the present invention, there is provided a method of spatially barcoding one or more detection probes, comprising:
(a) Providing a tissue with one or more detection probes bound to one or more biological molecules of interest, wherein the or each detection probe may comprise a photocleavable group;
(b) Optionally, if the or each detection probe does not comprise a photocleavable group, adding a photocleavable group to the or each detection probe;
(c) Illuminating an area of interest within the tissue to be spatially barcoded, wherein the illumination cleaves or alters the photocleavable group of the or each detection probe within the area;
(d) Adding an index sequence of the spatial barcode to the or each detection probe within the area illuminated in step (b), wherein the index sequence comprises a photocleavable group;
(e) Repeating steps (c) and (d) until the desired index sequences are added to form a spatial barcode attached to the or each detection probe within the area of interest.

In one embodiment, the biological molecules are selected from: nucleic acids, proteins, post-translational protein modifications, metabolites, small bioactive molecules, nucleotides, or drugs.

In one embodiment, the one or more detection probes are bound to more than one different type of biological molecule.

In one embodiment, the or each detection probe comprises a binding region to bind to a biological molecule. In one embodiment, the binding region may be an aptamer, nucleic acid, nucleic acid mimic, protein, or a mixture thereof.

In one embodiment, the method of the second aspect may comprise a step prior to step (a) of contacting a tissue with one or more detection probes to allow the or each detection probe to bind to one or more biological molecules of interest.

According to a third aspect of the present invention, there is provided a method of analysing one or more transcripts in a tissue, comprising:
(a) Contacting the tissue with one or more detection probes to allow the or each detection probe to bind to a transcript of interest, wherein the or each detection probe comprises a photocleavable group;
(b) Optionally, if the or each detection probe does not comprise a photocleavable group, adding a photocleavable group to the or each detection probe;
(c) Illuminating an area of interest within the tissue to be spatially barcoded, wherein the illumination cleaves or alters the photocleavable group of the or each detection probe within the area;
(d) Adding an index sequence of the spatial barcode to the or each detection probe within the area illuminated in step (b), wherein the index sequence comprises a photocleavable group;
(e) Repeating steps (c) and (d) until the desired index sequences are added to form a spatial barcode attached to the or each detection probe within the area of interest;
(f) Sequencing the one or more spatially barcoded detection probes of step (d) or a derivative thereof.

Suitably any number of transcripts of interest are analysed in the method, suitably one or more transcripts of interest are analysed in the method. In some cases, the entire transcriptome in a tissue may be analysed. In one embodiment, the transcript is RNA, suitably mRNA.

In one embodiment, the method of the third aspect is a method of analysing the transcriptome of a tissue. Suitably therefore, in step (a), the or each detection probe binds to the polyA region of a transcript of interest. Suitably the binding region of the or each detection probe binds to the poly-A region of the or each transcript of interest. Suitably, a detection probe binds to the polyA region of each transcript in the tissue. Suitably therefore each transcript in the tissue is spatially barcoded and subsequently sequenced.

In such an embodiment, the method of the third aspect may further comprise a step of elongating the or each detection probe. Suitably elongating the or each detection probe at the 3′ end. Suitably by reverse transcription. Suitably using any known reverse transcriptase enzyme. Suitably the elongation step produces one or more elongated detection probes wherein the or each modified detection probe comprises, in addition to the elements described hereinbelow, a nucleic acid sequence which is complementary to the transcript of interest, suitably at the 3′ end. Such a sequence may be termed the ‘elongated region’.

Suitably this step takes place between any of the steps of the method prior to the sequencing step. Suitably it takes place between steps (a) and (b) above. Alternatively this step may be performed between steps (e) and (f) above.

Suitably, in addition, the elongation step may further comprise the addition of a sequencing element to the 3′ end of the or each detection probe. Suitably the addition of a 3′ primer, or a 3′ sequencing adaptor. Suitably the addition of the sequencing element is carried out by template switching of reverse transcription. Suitably, the addition of the sequencing element can be also carried out by ligation, optionally following fragmentation of the elongated detection probe, or by PCR. Suitably ligation is carried out when the element is a primer or an adapter. Suitably PCR is carried out when the element is a primer, suitably random hexamer primers comprising a 5′ sequencing element.

In one embodiment, the or each detection probe comprises a binding region, wherein the binding region is a nucleic acid, or a nucleic acid mimic.

According to a fourth aspect of the present invention, there is provided a method of analysing one or more markers in a tissue, comprising:
(a) Contacting the tissue with one or more detection probes to allow the or each detection probe to bind to a marker of interest, wherein the or each detection probe comprises a photocleavable group;
(b) Optionally, if the or each detection probe does not comprise a photocleavable group, adding a photocleavable group to the or each detection probe;
(c) Illuminating an area of interest within the tissue to be spatially barcoded, wherein the illumination cleaves or alters the photocleavable group of the or each detection probe within the area;
(d) Adding an index sequence of the spatial barcode to the or each detection probe within the area illuminated in step (b), wherein the index sequence comprises a photocleavable group;
(e) Repeating steps (c) and (d) until the desired index sequences are added to form a spatial barcode attached to the or each detection probe within the area of interest;
(f) Sequencing the one or more spatially barcoded detection probes of step (d) or a derivative thereof.

In one embodiment, the or each marker is a biological molecule. In one embodiment, the or each marker is selected from: proteins, post-translational protein modifications, metabolites, small bioactive molecules, nucleotides, or drugs. In one embodiment, the or each marker is a protein, in such an embodiment, the method may be a method of analysing one or more proteins in a tissue. Suitably any number of proteins of interest are analysed in the method, suitably one or more proteins of interest are analysed in the method. In some cases, the entire proteome in a tissue may be analysed.

In one embodiment, the or each detection probe comprises binding region, wherein the binding region is a protein, aptamer, nucleic acid, nucleic acid mimic or a mixture thereof. In one embodiment, the binding region is an antibody or a nanobody.

According to a fifth aspect of the present invention, there is provided a method of analysing one or more transcripts and one or more markers in a tissue, comprising:
(a) Contacting the tissue with a plurality of detection probes to allow the detection probes to bind to both a nucleic acid and a marker of interest in the tissue, wherein the or each detection probe comprises a photocleavable group;
(b) Optionally, if the or each detection probe does not comprise a photocleavable group, adding a photocleavable group to the or each detection probe;
(c) Illuminating an area of interest within the tissue to be spatially barcoded, wherein the illumination cleaves or alters the photocleavable group of the or each detection probe within the area;
(d) Adding an index sequence of the spatial barcode to the or each detection probe within the area illuminated in step (b), wherein the index sequence comprises a photocleavable group;
(e) Repeating steps (c) and (d) until the desired index sequences are added to form a spatial barcode attached to the or each detection probe within the area of interest;
(f) Sequencing the one or more spatially barcoded detection probes of step (d) or derivatives thereof.

In one embodiment, the one or more markers that are analysed in addition to the one or more transcripts are selected from: proteins, post-translational protein modifications, metabolites, small bioactive molecules, nucleotides, or drugs. In one embodiment, the one or more markers are proteins. In one embodiment, the method may comprise a method of analysing the transcriptome and the proteome in a tissue.

In one embodiment, the plurality of detection probes comprises: one or more detection probes comprising a binding region which is a nucleic acid, nucleic acid mimic, or aptamer, and one or more detection probes comprising a binding region which is a protein. In one embodiment, the protein binding region is an antibody or a nanobody.

In one embodiment, any of the methods described in the first to the fifth aspects of the invention may further comprise a step of assigning a unique spatial barcode to each location or area of interest. Suitable locations or areas are defined elsewhere herein. Suitably this step occurs prior to step (c).

In one embodiment, the methods of the third, fourth or fifth aspects may further comprise a step of preparing the or each spatially barcoded detection probe for sequencing. Suitably this step occurs prior to step (f). Suitable steps for preparing the or each spatially barcoded detection probe are defined elsewhere herein.

In one embodiment, in the methods of the first to fifth aspects of the invention where the biological molecule is a nucleic acid, the method may further comprise a step of pre-amplification. Suitably a step of pre-amplification of the nucleic acids of interest. Suitably therefore the methods may comprise a step (a) of amplifying one or more nucleic acids of interest from the tissue. Such amplification may be carried out by any known process such as by rolling circle amplification. For example rolling circle amplification on circularised DNA molecules produced using starMAP, padlock probes, circLigase, and/or splint ligation. Optionally the circularisation step may be followed by a processing step, suitably a DNA polymerisation step, suitably by any strand displacing DNA polymerase such as phi29. Suitably in such embodiments, the step of contacting with a plurality of detection probes to allow the detection probes to bind to the or each nucleic acid of interest is performed on the product of the amplification. Suitably such a step may comprise contacting the product of the amplification step with a plurality of detection probes to allow the or each detection probe to bind to the product of amplification. Suitably the product of the amplification includes a plurality of copies of a nucleic acid sequence complementary to each nucleic acid of interest, and a plurality of copies of a unique DNA sequence assigned to each nucleic acid of interest. Suitably the unique DNA sequence is targeted and bound by the or each detection probe(s). Suitably in embodiments where the amplification comprises rolling circle amplification, the amplification product may comprise a DNA concatemer. Suitably the DNA concatemer comprises multiple copies of a nucleic acid sequence complementary to the nucleic acid of interest and multiple copies of a unique DNA sequence, to which the or each detection probe binds.

In one embodiment, in the methods of the first to fifth aspects of the invention where the biological molecule is a nucleic acid, the method may comprise the use of split detection probes. Suitably each split detection probe comprises a first part and a second part. Suitably both the first and second parts bind to a given nucleic acid sequence of interest. Suitably the first and second parts form a whole detection probe upon binding to the nucleic acid sequence of interest and annealing to each other. Suitably the first and second parts form a whole detection probe upon binding to the nucleic acid sequence of interest within annealing distance from each other. A suitable annealing distance may be between 1-100 nucleotides. Suitably the first and second parts form a whole detection probe upon binding to the nucleic acid sequence of interest by annealing to each other. Suitably both the first and second parts of the probe must bind to a nucleic acid sequence of interest within annealing distance of each other in order for the whole detection probe to form, and for the index sequences to be successfully added. Suitably step (d) comprising the addition of the index sequence is dependent on formation of a whole detection probe in step (a).

Suitably therefore step (a) of the methods may comprise contacting the tissue with one or more split detection probes to allow the or each split detection probe to bind to a nucleic acid of interest and form a whole detection probe, wherein the whole detection probe comprises a photocleavable group. Suitably wherein contacting the tissue with the split detection probes comprises contacting the tissue with first and second parts of each detection probe. Suitably wherein the first part and the second part of each split detection probe bind to the nucleic acid of interest. Suitably within annealing distance of each other, suitably at most 100 nucleotides from each other. Suitably wherein forming the whole detection probe comprises annealing of the first and second parts of the detection probe.

Suitably the pre-amplification step and the split detection probes may be used individually or together in the same method. Each of these embodiments increases the specificity of the method of the invention by decreasing background noise from non-specific binding of the detection probes.

In one embodiment, the or each index sequence used in the methods of the first to the fifth aspects is selected from the library of index sequences defined in the eighth aspect.

According to a sixth aspect of the present invention, there is provided a tissue produced by the process of the second, third, fourth or fifth, aspect, wherein the tissue comprises spatially barcoded detection probes.

According to a seventh aspect of the present invention, there is provided a detection probe comprising:
A binding region;
A species barcode; and
A photocleavable group

In one embodiment, the detection probe further comprises a unique molecular identifier (UMI).

In one embodiment, the detection probe further comprises an amplification region.

In one embodiment, the detection probe is suitable for binding to a target biological molecule present within the tissue.

In one embodiment, the biological molecule is selected from: an RNA transcript, a genomic DNA molecule, a protein, a post-transcriptional protein modification, a metabolite, a small bioactive molecule, a nucleotide, or a drug. In one embodiment, the biological molecule is the polyA region of an RNA transcript.

In one embodiment, the binding region is suitable for binding to a target biological a molecule within a tissue. In one embodiment, the binding region is a nucleic acid, a nucleic acid mimic, an aptamer, or a protein.

In one embodiment, the binding region is a nucleic acid, suitably a DNA molecule, suitably a DNA molecule with a complementary sequence to a given RNA transcript or other target DNA molecule. In one embodiment, the binding region is a DNA molecule with a complementary sequence to a polyA region in an RNA transcript.

In one embodiment, the binding region is a protein, suitably an antibody or a nanobody specific for a target marker, suitably a marker selected from: a protein, protein modification metabolite, bioactive molecule, nucleotide, or drug of interest.

In one embodiment, the detection probe comprises a binding region, and a nucleic acid sequence comprising a species barcode, and a photocleavable group. In one embodiment, the detection probe comprises a binding region attached to a nucleic acid sequence comprising a species barcode, and attached to a photocleavable group. Suitably the nucleic acid may further comprise a UMI and/or an amplification region.

In one embodiment, the detection probe comprises a binding region complementary to a polyA region in the RNA transcript, a nucleic acid sequence comprising a species barcode, and a photocleavable group. Optional elements include a unique molecular identifier (random DNA region), a polymerase promoter for amplification such as T7 promoter, and a sequencing element as explained above.

Suitably in such an embodiment, the detection probe may be a modified detection probe. Suitably the modified detection probe is for use in a method of the third aspect. Suitably in a method of analysing the transcriptome of a tissue.

In one embodiment, the modified detection probe may be elongated during the method of the invention, and may then further comprise a nucleic acid sequence which is complementary to a transcript of interest, suitably at the 3′ end. Suitably this additional nucleic acid sequence may be termed an elongation region, and is present at the 3′ end of the binding region of the detection probe after a step of elongation.

According to an eighth aspect of the present invention there is provided a library of index sequences, wherein each index sequence comprises:
a total length of between 5 and 50 nucleotides; and
a photocleavable group bound to one or both of the 5′ or 3′ ends of the molecule.

In one embodiment, each index sequence comprises blunt ends. In an alternative embodiment, each index sequence comprises overhangs at the 5′ and 3′ end thereof. Suitable overhangs are defined elsewhere herein.

In one embodiment, the index sequences are nucleic acid sequences. Suitably comprising a 5′ and a 3′ end. Suitably, the index sequences may be RNA, DNA, or modified backbone nucleic acid sequences, comprised of canonical or non-canonical bases. In one embodiment, the index sequences are DNA. Suitably the DNA is double stranded with the exception of the overhangs if present.

According to a ninth aspect of the present invention, there is provided a spatial barcode comprising a plurality of index sequences, wherein the index sequences are selected from the library of the seventh aspect.

In one embodiment, the spatial barcode comprises between 1 to 50 index sequences. In one embodiment, the spatial barcode is between 10 nucleotides and 250 nucleotides in length. In one embodiment, the index sequences are linked to each other, suitably by a chemical bond, suitably the chemical bond is compatible with processing by DNA and RNA polymerases. In one embodiment, the plurality of index sequences are linked together by phosphodiester bonds.

According to a tenth aspect of the present invention, there is provided a spatially barcoded detection probe comprising a detection probe attached to a spatial barcode as defined in the ninth aspect.

In one embodiment, the detection probe is as defined in the seventh aspect.

According to an eleventh aspect of the present invention there is provided a kit comprising a library of index sequences as defined in the eighth aspect, one or more detection probes as defined in the seventh aspect, optionally a ligase enzyme, and optionally one or more reagents.

In one embodiment, the one or more reagents include: one or more buffers, one or more sequencing reagents, one or more hydrogel monomers.

According to a twelfth aspect of the present invention, there is provided a system for spatial barcoding, the system comprising:
an instrument for viewing a substrate;
a light source for illuminating one or more locations of the substrate;
a microfluidic circuit for delivering one or more index sequences and reagents to the substrate
a processor for implementing software operable to control the instrument, light source, and microfluidic circuit.

In one embodiment, the substrate is a tissue, as described above. In one embodiment, the system is for spatially barcoding one or more detection probes, and/or spatially barcoding one or more markers. Suitably in one or more areas of interest. Suitably in one or more areas of interest of a tissue.

Further features and embodiments of the above aspects will now be defined in the following sections. Each feature may be combined in any order or in any combination with any of the above aspects.

The term ‘nucleic acid’ as used herein refers to any polymer formed of a plurality of nucleotide bases, wherein the bases may be comprised of canonical or non-canonical bases, and wherein the backbone may be modified or unmodified, and wherein the nucleotides may be linked by conventional phosphodiester bonds, or non-conventional bonds such as phosphorothioate bonds or chemical bonds. The term ‘nucleic acid mimic’ as used herein refers to a nucleic acid which is non-natural in some manner, for example, wherein one or more of the nucleotide bases is non-canonical, or wherein the backbone is modified, or wherein the bases are non-conventionally linked. ‘nucleic acids’ and ‘nucleic acid mimics’ may include: bridged nucleic acids, locked nucleic acids, peptide nucleic acids, traditional DNA and RNA, for example.

The term ‘a’ or ‘an’ as used herein may refer to the relevant feature in the singular or plural, and should be taken to mean at least one of the relevant feature, and may refer to one or more of the relevant feature.

Tissue

Some aspects of the present invention involves in situ analysis of gene expression or protein/marker abundance within a biological tissue. The first step of the methods of the invention is to label or provide a labelled tissue with detection probes that bind or are prebound to biological molecules of interest within the tissue.

Suitably the tissue may be from any living source.

Suitably the tissue may be from a human or animal source.

Suitably the tissue may be diseased or healthy tissue.

Suitably the tissue is a sample of tissue. Suitably the sample of tissue is a section. Suitably the section may be obtained by any known means such as a microtome, cryostat, cryomicrotome or vibratome.

Suitably, the tissue section has a thickness ranging from 3 μm to 100 μm. In other embodiments, the tissue section may be thicker depending upon the ability to deliver the required amount of illumination in the area or location of interest with which to cleave or alter the photocleavable groups therein.

Suitably the tissue may be a monolayer of cells.

Suitably the tissue may be stained with one or more stains. Suitably the stains may be any stains known in the art of preparing tissue samples. Suitable stains may include nuclear and/or membrane stains. For example: eosin, DAPI, hematoxylin, phalloidin, WGA, and the like.

Suitably, the tissue may be subjected to one round of immunohistochemistry, or in situ hybridisation according to any method known in the art, for the purpose of visualizing the distribution of certain protein markers using fluorescence imaging. Suitably the methods may comprise a step of staining the tissue. Suitably the methods may comprise a step of immuno-staining the tissue. Suitably the round of immunohistochemistry may be carried out prior to step (a) of the methods of the invention.

Suitably, prior to the methods of the invention, the tissue or substrate is imaged. Suitably therefore the methods may comprise a step of imaging the tissue or substrate. Suitably the tissue is imaged by a camera. Suitably the camera captures one or more images of the tissue.

Optionally the camera may be part of the instrument, suitably the microscope, used to image the tissue.

Suitably software is used to analyse the one or more images of the tissue. Suitably the software described in the twelfth aspect of the invention is operable to analyse one or more images of the tissue. Suitably the software is operable to conduct image analysis. Suitably the software is operable to conduct mosaic imaging analysis. Suitably therefore the method may comprise a step of conducting mosaic imaging analysis of one or more images of the tissue or substrate. Suitably the software is operable to identify individual cells or sub-cellular regions within the or each image, suitably by automated object recognition. Suitably therefore the method may comprise identifying individual cells or sub-cellular regions in one or more images of the tissue. Suitably, the software allows a user to select any number of locations or areas of interest for subsequent spatial barcoding. Suitably therefore the method may comprise a step in which one or more locations or areas of interest are selected, suitably from the one or more images, for spatial barcoding.

In some aspects of the invention, the method involves spatially barcoding one or more locations. In one embodiment, the one or more locations are on a substrate. Suitably the substrate may be an inert substrate such as glass, plastic, etc. Suitably the inert substrate may be a slide, plate, mount, tube, or other item for conducting an assay. Alternatively, the substrate may be living, suitably the substrate may be tissue. Suitably the tissue may be as defined herein. Any features defined herein in relation to an area of the tissue, may equally apply to a location on a substrate.

Area or Location of Interest

The methods of the present invention comprise selecting and illuminating one or more locations or areas of interest, in which locations or areas detection probes or root molecules are to be spatially barcoded.

Suitably, reference to ‘area’ or ‘location’ herein may refer to a two-dimensional region or a three-dimensional region. Suitably to a region of any size. Suitably the maximum size of the region may be determined by the properties of the illumination and/or the particular tissue or substrate used in the method.

Suitably a location of interest may be any region, suitably any region on a substrate. Suitably, a location of interest is a two-dimensional region.

Suitably a location of interest may be between 1 μm²-150 mm² in size, suitably between 1 μm²-1 mm² in size, suitably between 1 μm²-1,000,000 μm² in size, suitably between 1 μm²-200,000 μm² in size, suitably between 1 μm²-20,000 μm² in size, suitably between 1 μm²-1000 μm² in size.

Suitably an area of interest may be any region within a tissue. Suitably, an area of interest is a three-dimensional region within the tissue.

Suitably an area of interest may be between 1 μm³-150 mm³ in size, suitably between 1 μm³-1 mm³ in size, suitably between 1 μm³-1,000,000 um³ in size, suitably between 1 μm³— 200,000 μm³ in size, suitably between 1 μm³-20,000 μm³ in size, suitably between 1 μm³- 1000 μm³ in size.

Suitably an area or location of interest may be a collection of cells, suitably an area or location of interest may comprise from 1 up to 100,000,000 cells, 1,000,000 cells, 1000 cells, 100 cells, 10 cells. Suitably an area or location of interest may comprise a single cell. Suitably an area or location of interest may comprise a sub-cellular region or compartment.

Suitably one or more locations or areas of interest are pre-selected, suitably prior to the methods of the invention. Suitably a user selects the locations or areas of interest, suitably from an image of the tissue. Suitably an area or location may be selected based on pixels or based on features of the image, or both. Suitably image processing aids selection of an area or location from an image.

Suitably software then assigns a unique spatial barcode to each selected location or area of interest. Suitably therefore, the methods of the invention may comprise a step of selecting one or more locations interest of the substrate, or selecting one or more areas of interest of the tissue. Suitably therefore, the methods of the invention may comprise a step of assigning a spatial barcode to each selected location or area of interest.

Suitably multiple locations or areas of interest can be selected. Suitably the locations or areas of interest do not have to be contiguous.

Suitably the number of locations or areas that can be selected is determined by the number of possible unique spatial barcode sequences. The number of unique spatial barcode sequences is in turn determined by the number of different index sequences used and by the number of index sequences included in each spatial barcode.

Suitably, based on a method using 4 different index sequences and 10 index sequences per spatial barcode, up to 1 million locations or areas of interest can be selected.

Detection Probe

The present invention makes use of detection probes which bind to biological molecules in the tissue. The detection probes may be prebound to biological molecules in the tissue, or they may be contacted with the tissue as part of the method of spatial barcoding.

In the methods which comprise a step of contacting a tissue with one or more detection probes to allow the or each detection probe to bind to a biological molecule, suitably the contact is under conditions sufficient to allow binding of the or each detection probe to a biological molecule.

Suitable conditions may include contacting the tissue with the or each detection probe for a sufficient length of time to allow binding to the biological molecule. Suitably a sufficient time is between 1 h and 1 week depending on the size of the tissue sample.

Suitable conditions may include contacting the tissue with a sufficient concentration of the or each detection probe to allow binding to the biological molecule. Suitably a sufficient concentration is between 1 pM and 1 μM depending on the abundance of the biological molecule and the number of detection probes.

Suitable conditions to allow binding of a given detection probe to a given biological molecule of interest will be known or determined by the skilled person.

Suitably the detection probe for use in the methods of the invention may be any probe which is suitable for in situ hybridization methods. Suitably the or each detection probe may comprise, for example, a binding region, a species barcode, optionally a unique molecular identifier (UMI), and, optionally, any the following elements: a nucleic acid sequence complementary to the transcript of interest, a photocleavable group and an amplification region. Suitably therefore, any detection probe known in the art could be used, as long as it comprises a binding region and a species barcode, and, optionally, any the following elements: a nucleic acid sequence complementary to the transcript of interest, a UMI, a photocleavable group and/or an amplification region. In some embodiments, the or each detection probe may comprise a binding region which comprises a species barcode, or alternatively a binding region which also functions as a species barcode.

Suitably, the detection probe for use in the methods of the invention may be any probe which is suitable for immunohistochemistry methods, which further comprises a binding region and a species barcode, optionally a unique molecular identifier (UMI), and, optionally, any the following elements: a nucleic acid sequence complementary to the transcript of interest, a photocleavable group and an amplification region. Suitably therefore, any immunohistochemistry detection probe known in the art could be used, as long as it comprises a binding region and a species barcode, and optionally, any the following elements: a unique molecular identifier (UMI), a nucleic acid sequence complementary to the transcript of interest, a photocleavable group and/or an amplification region.

Suitably the detection probes may not comprise photocleavable groups, in which case the methods comprise step (b) of adding a photocleavable group to the or each detection probe.

In one embodiment, one or more detection probes of the invention are used in the methods of the invention.

A detection probe of the invention comprises:
A binding region;
A species barcode; and
A photocleavable group

In one embodiment, the detection probe further comprises a unique molecular identifier (UMI).

In one embodiment, the detection probe further comprises an amplification region.

In one embodiment, the detection probe further comprises a sequencing element.

Suitably the binding region allows the detection probe to bind to a biological molecule. Suitably wherein the biological molecule is present in the tissue. Suitably the binding region allows the detection probe to bind to a nucleic acid, or to a protein, post-translational protein modification, metabolite, small bioactive molecule, nucleotide, or drug. Suitably the binding region comprises a nucleic acid, nucleic acid mimic, aptamer, or a protein.

In an embodiment where the biological molecule is a nucleic acid, i.e. a RNA transcript or a DNA molecule of interest, suitably the binding region is a nucleic acid or nucleic acid mimic. Suitably the nucleic acid is capable of hybridising to the nucleic acid of interest. Suitably the binding region is DNA. Suitably the binding region is DNA capable of hybridising to a RNA transcript of interest. In one embodiment, the biological molecule may be an RNA transcript. In one embodiment, the biological molecule is the polyA region of an RNA transcript. In such an embodiment, the binding region is a nucleic acid, suitably DNA, capable of hybridising to the polyA region of a RNA transcript of interest.

In one embodiment, the detection probe may be split. Suitably each split detection probe comprises a first part and a second part. Suitably both the first and second parts bind to a given nucleic acid sequence of interest. Suitably the first and second parts form a whole detection probe upon binding to the nucleic acid sequence of interest, and annealing to each other. Suitably both the first and second parts of the probe must bind to a nucleic acid sequence of interest and anneal together in order for the whole detection probe to form, and for the index sequences to be successfully added. Suitably the first and second parts together comprise the features of the detection probe described above.

Suitably, the first part of the split detection probe comprises: a binding region, a species barcode, a region for annealing to the second part, and a photocleavable group. Suitably, the first part of the split detection probe can also optionally include a unique molecular identifier (random DNA region), a polymerase promoter for amplification such as T7 promoter, and a sequencing element. Suitably the second part of the split detection probe comprises: a binding region, and a region for annealing to the first part.

In one embodiment there is provided a split detection probe comprising a first part and a second part, wherein the first part comprises:
a binding region;
a species barcode;
a region capable of annealing to the second part; and
a photocleavable group;

wherein the second part comprises:
a binding region; and
a region capable of annealing to the first part.

In one embodiment, the first part further comprises a unique molecular identifier (UMI).

In one embodiment, the first part further comprises an amplification region.

In one embodiment, the first part further comprises a sequencing element.

Suitably other features of the split detection probe are the same as described herein for the typical detection probe.

Suitably the binding region of both the first and second parts is capable of binding to a nucleic acid of interest, suitably the same nucleic acid of interest. Suitably the binding region of the first part is capable of binding to a nucleic acid of interest within an annealing distance of the second part. Suitable annealing distance may be less than 100 nucleotides, suitably less than 50 nucleotides, suitably less than 20 nucleotides, suitably less than 10 nucleotides, suitably less than 5 nucleotides.

Suitably when the first part and the second part are bound to a nucleic acid sequence of interest within annealing distance, the region of the first part capable of annealing to the second part anneals to the region of the second part capable of annealing to the first part. Suitably a whole detection probe is formed to which an index sequence can bind.

Suitably, the binding region is linked to the remaining components of the detection probe by a covalent bond.

In an embodiment where the biological molecule is a marker, for example a protein of interest, a post-translational protein modification, a metabolite, a small bioactive molecule, a nucleotide or a drug, suitably the binding region is a protein or aptamer. Suitably the protein or aptamer is capable of specifically binding to the marker of interest. Suitably the binding region is an antibody, Fab, single-chain antibody, nanobody or the like.

Suitably, the binding region is linked to the remaining components of the probe by a covalent bond.

In one embodiment, the detection probe comprises a binding region, and a nucleic acid sequence comprising at least a species barcode, and a photocleavable group. In one embodiment, the detection probe comprises a binding region linked to a nucleic acid sequence comprising at least a species barcode, and a photocleavable group. Suitably the nucleic acid may further comprise a UMI and/or an amplification region.

In one embodiment, the detection probe comprises a binding region and a nucleic acid sequence linked thereto, wherein the nucleic acid sequence comprises: a species barcode, a UMI, an amplification region, and a photocleavable group.

In one embodiment, the detection probe comprises a binding region complementary to a polyA region in the RNA transcript, a nucleic acid sequence comprising a species barcode, and a photocleavable group. Optional elements include a unique molecular identifier (random DNA region), a polymerase promoter for amplification such as T7 promoter, and a sequencing element as explained above.

Suitably in such an embodiment, the detection probe may be a modified detection probe. Suitably the modified detection probe is for use in a method of the third aspect. Suitably in a method of analysing the transcriptome of a tissue.

In one embodiment, the modified detection probe may be elongated during the method of the invention, and may then further comprise a nucleic acid sequence which is complementary to a transcript of interest, suitably at the 3′ end. Suitably this additional nucleic acid sequence may be termed an elongation region, and is present at the 3′ end of the binding region of the detection probe after a step of elongation.

Suitably the binding region may be linked to the remaining components, which may comprise a nucleic acid sequence, by a covalent bond. Suitably, when the binding region comprises a nucleic acid itself, it is linked to the remaining components, which may comprise a nucleic acid sequence, by a phosphodiester bond. Suitably, when the binding region comprises a protein, it is linked to the remaining components, which may comprise a nucleic acid sequence, by a chemical bond.

Suitable means for linking proteins, such as a binding region protein, with nucleic acid sequences for forming detection probes are known in the art. Suitably a linker may be used.

Suitably the amplification region is a nucleic acid or nucleic acid mimic. Suitably the amplification region is DNA.

Suitably the amplification region comprises a promoter for a polymerase. Suitably the promoter is for an RNA polymerase. In one embodiment, the promoter is the T7 RNA polymerase promoter or that of another single subunit polymerase.

Suitably the species barcode is also a nucleic acid or nucleic acid mimic. Suitably the species barcode is DNA. Suitably, the species barcode is separate from the spatial barcode of the invention. Suitably the species barcode allows identification of the biological molecule that the detection probe binds to. Suitably, during sequencing, the species barcode identifies the biological molecule that the detection probe was bound to in the tissue.

Suitably the UMI is also a nucleic acid or nucleic acid mimic. Suitably the UMI is DNA. Suitably the UMI is unique to each detection probe. Suitably the combination of the individual UMI and each detection probe molecule is unique. Suitably therefore, the UMI allows quantification of the detection probes by counting the number of different UMI sequences. Suitably the UMI thereby facilitates quantification of the biological molecule that the probe binds to. Suitably, during sequencing, the UMI identifies the detection probe and allows collapsing of reads that represent a single event of a detection probe binding to its target biological molecule. Suitably the number of different detection probe molecules bound to a biological molecule gives an indication of the expression of that biological molecule.

Suitably the photocleavable group is defined elsewhere herein.

Suitably the or each detection probe may further comprise a stabiliser. Suitably the stabiliser is a nucleic acid or nucleic acid mimic. Suitably a double-stranded nucleic acid. Suitably the stabiliser produces a double-stranded region compatible with dsDNA ligase enzymes. Suitably the stabiliser is between 4 and 50 nucleotides in length. Suitably, in some embodiments where a split detection probe is used, a stabiliser is present. Suitably, the stabiliser is formed by annealing between the first part and second part of the detection probe to form a double stranded region. Suitably the first part and the second part of the detection probe anneal to form a stabiliser if they are both bound to a nucleic acid sequence of interest. Suitably such annealing of the first part and second part forms a whole detection probe as described above. Suitably the first part and the second part of the detection probe must be bound to the nucleic acid sequence within annealing distance of each other for this to occur. Suitably within 1 nucleotide and 100 nucleotides, suitably between 1 and 50, suitably between 1 and 20 nucleotides of each other.

Suitably the or each detection probe may further comprise one or more sequencing elements. Suitably the or each sequencing element aids later sequencing of the detection probe. Suitably at least one of the sequencing elements is a primer. Suitably a primer for sequencing library amplification. Suitably the primer is a forward primer, suitably a forward primer used for a sequencing library amplification.

In one embodiment, the detection probe may comprise the following structure:

3′-[binding region]-[nucleic acid sequence]-[photocleavable group]-5′

Wherein the nucleic acid sequence comprises at least a species barcode, and optionally an amplification region, UMI, sequencing element, and stabiliser.

In one embodiment, therefore, the detection probe may comprise the following structure:

3′-[binding region]-[amplification region]-[species barcode]-[UMI]-[stabiliser]-[photocleavable group]-5′

In one embodiment, the detection probe may be a modified detection probe, and may comprise the following structure:

3′-[binding region complementary to a polyA region in the transcript]-[nucleic acid sequence]-[photocleavable group]-5′

Wherein the nucleic acid sequence comprises at least a species barcode, and optionally an amplification region, UMI, sequencing element and stabiliser.

Suitably wherein the binding region is a nucleic acid.

Root Molecule

In the first aspect of the invention, a method of spatially barcoding one or more locations of a substrate is recited in which the first step comprises binding one or more root nucleic acid molecules to the or each location on which the spatial barcode will be constructed.

Suitably the root molecule is a nucleic acid or nucleic acid mimic, suitably the root molecule is DNA. Suitably the root molecule comprises a first end and a second end. Suitably a first end of the root molecule is able to bind to a substrate. Suitably a second end of the root molecule is able to bind to a bridge molecule or an index sequence.

Suitably the root molecule may comprise a photocleavable group, suitably at the non-bound end thereof, suitably at the second end thereof. Suitably when the root molecule comprises a photocleavable group, it is able to bind to an index sequence and step (b) of the method is not required.

Suitably the or each root molecule may comprise the same features as a detection probe, however the binding region is suitable for binding to a substrate.

Bridge Molecule

The methods of the invention require the presence of a photocleavable group on the or each of the root nucleic acid molecules or detection probes bound to a specimen that is subjected to the method. The photocleavable group allows control of which root nucleic acid molecules or which detection probes, and later which index sequences, are available for further index sequences to be added. In this way, the photocleavable groups allow control of where and when spatial barcodes are formed.

Suitably, the photocleavable group can either be a component of the or each root molecule or detection probe, or it can be added onto the or each root molecule or detection probe.

Suitably, a photocleavable group may be added to the or each root molecule or detection probe by the addition of a molecule defined as a bridge. The use of a bridge molecule is advantageous in that it allows a large diversity of root molecules or detection probes to be used on the specimen, without the need to modify each different molecule with a photocleavable group during chemical synthesis. This reduces the cost and complexity involved in the production of a library of detection probes or root molecules which may be used in the methods of the invention.

Suitably, the bridge molecule is a nucleic acid or mimic. Suitably the bridge molecule is DNA.

Suitably, the DNA bridge molecule is a double stranded DNA molecule. Suitably the bridge molecule is between 5 and 40 nucleotides in length and comprises a photocleavable group at the 5′ end or the 3′ end of the molecule, or both.

Suitably, in the cases in which a photocleavable group is not already present on the root molecule or on the detection probe, a photocleavable group is added to the or each root molecule or detection probe in step (b) of the methods.

Suitably, a photocleavable group may be added to a library of detection probes, or a library of root molecules before they are used in the methods of the invention. Suitably before the library is contacted with the substrate or tissue sample.

Alternatively, a bridge molecule may be added to the or each of the root molecules or the or each of the detection probes in step (b) of the methods of the invention. Suitably the bridge molecule is added to the or each root molecule or the or each detection probe by ligation. Suitably ligation of the bridge molecule is carried out by the same process of ligation as for the index sequences. Suitably by a ligase enzyme. Suitable ligases are described elsewhere herein.

Suitably, the bridge molecule may further comprise one or more sequencing elements, or purification elements to aid purification of the or each detection probe or root molecule. Suitably the or each sequencing element aids later sequencing of the or each root molecule or detection probe. Suitably at least one of the sequencing elements is a primer. Suitably the primer is a forward primer used for a sequencing library amplification.

Biological Molecule

The methods of the invention allow the situ analysis of the expression of markers or biological molecules in a tissue. In particular, the methods allow the spatial analysis of the expression of markers or biological molecules in a tissue.

In one embodiment, the or each marker is a biological molecule.

Suitably the one or more biological molecules can be any molecule indicative of gene expression.

Suitably the or each biological molecule may be selected from: a nucleic acid, a protein, a covalently modified nucleic acid, a covalently modified protein, a post-transcriptional protein modification, a metabolite, a small bioactive molecule, a nucleotide, and a drug.

Suitably the or each biological molecule may be a transcript, suitably a mRNA molecule, large or small non-coding RNA, circular RNA, or other expressed transcript, including alternatively spiced forms of mRNAs. Suitably, the or each biological molecule may be a covalently modified transcript bearing a modifying chemical group.

In one embodiment, the or each biological molecule is an RNA transcript.

Suitably, the or each biological molecule may be a DNA molecule, suitably a genomic DNA molecule or a heterologous DNA molecule. Suitably the or each biological molecule may be a circular DNA molecule or a DNA concatemer. Suitably, the or each biological molecule may be a covalently modified DNA molecule bearing a modifying chemical group, suitably a methyl, hydroxymethyl or formyl group.

Suitably, the or each biological molecule may be a protein, suitably a polypeptide.

Suitably, the or each biological molecule may be a post-translationally modified protein bearing a post-transcriptional modification known in the art, for instance a glycosylation, phosphorylation, acetylation, or the like.

Suitably, the or each biological molecule may be a metabolite, a small bioactive molecule, a nucleotide or nucleoside, a chemically modified nucleotide or nucleoside, or a drug.

Suitably the methods of the invention may allow analysis of one or more transcripts in a tissue, suitably any number of transcripts of interest are analysed in the method, suitably one or more transcripts of interest are analysed in the method. In some cases, the entire transcriptome in a tissue may be analysed. Suitably in such methods the or each biological molecule is a nucleic acid, suitably a transcript, suitably mRNA.

Suitably the methods of the invention may allow analysis of one or more proteins in a tissue, suitably any number of proteins of interest are analysed in the method, suitably one or more proteins of interest are analysed in the method. In some cases, the entire proteome in a tissue may be analysed. Suitably in such methods the or each biological molecule is a protein or a post-translationally modified protein, suitably a polypeptide or covalently modified polypeptide

Suitably the methods of the invention may also allow analysis of one or more transcripts and one or more markers in a tissue. In one embodiment, the one or more markers that are detected and quantified in addition to the one or more transcripts are selected from: proteins, post-translational protein modifications, metabolites, small bioactive molecules, nucleotides, or drugs. In one embodiment, the one or more markers are proteins. Suitably in such methods a plurality of biological molecules are bound by the detection probes, suitably the plurality of biological molecules comprise both nucleic acids and one or more other type of marker.

Suitably the methods of the invention may also allow analysis of the transcriptome and proteome of a tissue, suitably in such methods a plurality of biological molecules are bound by detection probes, suitably the plurality of biological molecules comprise both nucleic acids and proteins, suitably both transcripts and polypeptides, or covalently modified transcripts and polypeptides.

Suitably the methods of the invention may also allow the detection of DNA molecules, their copy number, and the presence or absence of single nucleotide variants or the length of simple repeats.

Photocleavable Group

The present invention utilises root nucleic acid molecules, bridge molecules, detection probes and index sequences that each may comprise a photocleavable group. The photocleavable group allows control of which root molecules, which detection probes, and later which index sequences, are available for further index sequences to be added. In this way, the photocleavable groups allow control of where and when spatial barcodes are formed.

Suitably the or each root molecule may comprise a photocleavable group. Suitably the or each detection probe may comprise a photocleavable group. Suitably, the or each bridge molecule comprises a photocleavable group. Suitably each index sequence comprises a photocleavable group. In one embodiment, the or each detection probe comprises a photocleavable group. In one embodiment, the or each root molecule comprises a photocleavable group.

Alternatively, a photocleavable group may be added to the or each root molecule or the or each detection molecule by using a bridge molecule as described elsewhere herein.

Suitably the photocleavable group may be located at the 5′ end of the or each root molecule, bridge molecule, detection probe, or index sequence. Suitably, the photocleavable group may alternatively be located at the 3′ end of the or each root molecule, bridge molecule, detection probe, and index sequence.

Suitably the photocleavable group may be bound to the 5′ phosphate of the or each root molecule, bridge molecule, detection probe, and index sequence.

Suitably, the photocleavable group may be bound to the 3′ hydroxyl of the or each root molecule, bridge molecule, detection probe and index sequence.

Suitably the photo-cleavable group is a light-sensitive group which protects the 5′ or 3′ end of a nucleic acid sequence. Suitably the photo-cleavable group protects the 5′ or 3′ end of a nucleic acid sequence from addition of further nucleic acid sequences, suitably in the context of the present invention, the photocleavable group prevents the addition of an index sequence.

In one embodiment of the invention, the photocleavable groups when present, prevent a reaction from occurring, and when removed or altered permit a reaction to occur.

Suitably the photocleavable group prevents any hybridisation or ligation of nucleic acids to a root molecule, bridge molecule, detection probe or index sequence. Suitably, in the case of a root molecule, bridge molecule, or detection probe, the photocleavable group prevents ligation of an index sequence thereto. Suitably, in the case of an index sequence, the photocleavable group prevents hybridisation or ligation of a further index sequence thereto.

Suitably the photocleavable group comprises a cage. Suitably the cage protects the 5′ phosphate or the 3′ hydroxyl of a nucleic acid.

Suitably the photocleavable group is further attached to a fluorescent moiety. Suitably the fluorescent moiety allows detection of the photocleavable group and is suitably removed after removal or alteration of the photocleavable group.

Suitably, the photocleavable group may include a nitrobenzyl group, dimethoxy-nitrobenzyl group, nitrophenyl group, or nitroveratryl group.

Suitably the photocleavable group may be a PC-spacer or photocleavable spacer. Suitably the photocleavable spacer may comprise a structure according to formula I as noted in the examples.

Suitably the photocleavable group may be cleaved or altered by illumination. Suitably cleavage or alteration of the photocleavable group in response to illumination exposes the 5′ or 3′ end of the relevant nucleic acid. Suitably the cleavage or alteration of the photocleavable group allows the addition of further nucleic acid sequences, suitably index sequences, to the exposed 5′ or 3′ end of the nucleic acid; which may be a root molecule, a detection probe, a bridge molecule or an index sequence.

Suitably the photocleavable group may be altered by changing conformation in response to illumination, suitably by changing three-dimensional conformation in response to illumination.

Alternatively, the photocleavable group may be cleaved in response to illumination.

Suitably, the photocleavable group may be cleaved through a one-photon or two-photon mechanism. Suitably, in the one-photon mechanism, one single photon of light is on average absorbed by each photocleavable molecule resulting in photorelease. Suitably, illumination needed for this reaction is the range from 300 nm to 600 nm. Suitably, in the two-photon mechanism, two distinct photons of light are on average absorbed by each photocleavable molecule resulting in photorelease. Suitably, the two photons of light are absorbed within a femtosecond time period. Suitably, illumination needed for this reaction is in the range from 680 nm to 900 nm.

Suitable illumination which will act to cleave the photocleavable group is discussed elsewhere herein.

Illumination

The methods of the invention rely on illumination of selected locations or areas of interest in a sequential manner to control the order in which index sequences are added to detection probes bound in those areas. The order in which index sequences are added to the detection probes forms a unique spatial barcode corresponding to each location or area of interest.

Suitably illuminating a location or area of interest comprises illuminating a location or area of interest that has been selected by a user. Suitably the or each location or area of interest is selected by a user using software. Suitably this selection of locations or areas takes place prior to illuminating step (c)

Suitably illumination cleaves or alters photocleavable groups. Suitably illuminating a location or area of interest cleaves or alters the photocleavable groups present in that location or area. Suitably illuminating a location or area of interest cleaves or alters the photocleavable groups on the root molecules, the detection probes and/or the index sequences in that location or area. Suitably, in the first cycle of the methods, illuminating a location or area of interest cleaves or alters the photocleavable groups from each of the root molecules or detection probes in that location or area. Suitably, in subsequent cycles of the methods, illuminating a location or area of interest cleaves or alters the photocleavable groups from each of the bound index sequences in that location or area.

Suitably illumination cleaves or alters photocleavable groups from the root molecules, bridge molecules, detection probes and/or index sequences such that the 5′ end or 3′ end is exposed, and optionally available for reaction. Suitably illumination cleaves or alters photocleavable groups from the root molecules, bridge molecules, detection probes and/or index sequences such that the 5′ phosphate or 3′ hydroxyl is exposed, and optionally available for reaction. Suitably illumination allows the addition of an index sequence to the 5′ end or the 3′ end of the root molecules, bridge molecules, detection probes and/or index sequences.

Suitably illuminating an area of interest allows index sequences to be added to the root molecules, bridge molecules, detection probes and/or bound index sequences in that location or area. Suitably, in the first cycle of the methods, illuminating a location or area of interest allows an index sequence to be added to each of the root molecules, bridge molecules, or detection probes in the location or area. Suitably, in subsequent cycles of the methods, illuminating a location or area of interest allows a further index sequence to be added to each of the bound index sequences in the location or area.

Suitably, illumination determines in which locations or areas of interest a given index sequence will be added.

Suitably, multiple locations or areas of interest may be illuminated at once. Suitably, step (c) may comprise illuminating multiple locations or areas of interest.

Suitably therefore step (c) may comprise creating a pattern of illumination. Suitably therefore step (c) may comprise creating a pattern of illumination on the substrate or tissue, wherein the pattern of illumination comprises multiple locations or areas of interest. Suitably the same index sequence is added to each location or area of interest within a given pattern of illumination.

Suitably, the locations or areas of interest that are illuminated in step (c) change in each round of steps (c) and (d). Suitably therefore the pattern of illumination changes in each round of steps (c) and (d).

Suitably, in each ‘round’ of steps (c) and (d), all of the areas/locations of interest that have the same index sequence for that position are illuminated and the relevant index sequence is contacted and suitably added.

Suitably the methods comprise multiple rounds of steps (c) and (d) until each of the different index sequences is contacted to the areas/locations of interest, suitably added to the areas/locations, to fulfil the relevant position of the spatial barcode.

Suitably, a cycle is complete after one round has been performed for each of the different index sequences used in the spatial barcodes. Suitably a method using 4 different index sequences will have 4 rounds per cycle.

Suitably therefore a ‘cycle’ corresponds to completing a position of the spatial barcode for each area/location of interest. Suitably a ‘cycle’ corresponds to contacting the locations/areas with each of the index sequences to be used in the method.

Suitably the first cycle comprises a plurality of rounds of steps (c) and (d) to contact, suitably to add, the relevant index sequence corresponding to a first position in the spatial barcodes, to bound root molecules, bridge molecules and/or detection probes in the selected locations/areas.

Suitably after the first cycle, all index sequences in the first position of the allocated spatial barcodes have been contacted, suitably added.

Suitably the second cycle comprises a plurality of rounds of steps (c) and (d) to contact, suitably to add, the relevant index sequence, corresponding to a second position in the spatial barcodes, to bound index sequences in the selected locations/areas.

Suitably after the second cycle, all index sequences in the second position of the spatial barcodes have been contacted, suitably added.

Suitably any number of rounds per cycle may occur depending on the number of different index sequences to be used. Suitably any number of cycles may occur depending on the length of the spatial barcode to be added and therefore the number of index sequences comprised in each spatial barcode.

For example, each spatial barcode may comprise 10 positions and therefore 10 index sequences, and 4 different index sequences may be used in the method. Therefore the methods of the invention would comprise 4 rounds per cycle and 10 cycles in order to form the complete spatial barcodes.

Suitably, when referring to addition of ‘all’ index sequences in each cycle, and to ‘each’ of the different index sequences being added in a round, it will be appreciated that not every index sequence will always be added to every bound root molecules, bridge molecules and/or detection probes, or every bound index sequence. Some index sequences may not be added due to expected inefficiencies in the method, for example ligase enzymes are not 100% efficient.

Suitably in some cases, only some of the index sequences are added. Suitably, only some index sequences are added to the bound root molecules, bridge molecules and/or detection probes, or bound index sequences. Suitably, the index sequences are at least contacted with the relevant areas/positions for addition. Suitably a round or cycle is regarded as complete when all the required index sequences have been contacted with the relevant areas/locations.

Suitably illumination is not restricted to visible light, suitably use of the term ‘illumination’ of ‘illuminating’ herein refers to any wavelength of light, either visible or non-visible.

Suitably illumination of the or each location or area of interest is achieved by using a light source, suitably a light source of a constant wavelength, suitably by using a LED or a laser.

Suitably, illumination may be directed to each location or area of interest. Suitably by using a refractive or reflective optical system. Suitably the refractive or reflective optical system may have a resolution of 200 nm or above. Suitably the optical system may be comprised within a microscope, such as any microscope described in the art. Suitably the light source may also be comprised within a microscope. Suitably, the optical system includes an element to direct illumination to the or each location or area of interest. Suitably, the optical system includes an element to direct illumination from the light source to the or each location or area of interest.

In some embodiments, the element is a movable mirror, for example a galvanometric mirror. In some embodiments, the element is a digital micromirror device (DMD chip). In some embodiments, the element is a spatial light modulator.

Suitably the or each location or area of interest may be illuminated by light having a wavelength between 300-600 nm, suitably between 310 nm-570 nm, suitably between 320 nm-550 nm, suitably between 330 nm-520 nm, suitably between 340 nm-480 nm, suitably between 350 nm-450 nm, suitably between 360 nm-420 nm. Suitably, these wavelengths of light result in a one-photon photorelease process.

Alternatively, the or each location or area of interest may be illuminated by light having a wavelength between 680 nm and 900 nm, suitably between 700 and 850 nm, suitably between 720 and 800 nm. Suitably, these wavelengths of light result in a two-photon photorelease process.

Suitably the light may be UV or violet light or infrared light

In one embodiment, the or each location or area of interest is illuminated by light having a wavelength of between 350 nm-410 nm, for the one photon process, or 710 to 800 nm for the two-photon process. In one embodiment, the or each location or area of interest is illuminated with the same wavelength of light. Suitably the same wavelength of light is used throughout the methods of the invention.

Alternatively, a first location/area of interest may be illuminated by a first wavelength of light and a second location/area of interest may be illuminated by a second wavelength of light. Suitably, in this case one wavelength of light is in the 300 nm-450 nm range and a second wavelength of light is in the 500-600 nm range, using the one-photon photorelease process. Suitably the first and second locations/areas may be illuminated at the same time but by different wavelengths of light. Suitably, this may apply to multiple locations/areas of interest, which may be illuminated at the same time, but with different wavelengths of light.

Suitably each location/area of interest is illuminated with light of a sufficient power to cleave or alter the photocleavable groups in the given location or area. Suitably, each location/area of interest is illuminated with a light with an average power ranging from 10 mW/cm² to 30 W/cm², suitably from 20 mW/cm² to 20 W/cm², suitably from 50 mW/cm² to 10 W/cm², suitably from 100 mW/cm² to 5 W/cm², suitably from 200 mW/cm² to 1 W/cm². Suitably each location/area of interest is illuminated for a sufficient period of time to cleave or alter the photocleavable groups in that location/area. Suitably each location/area of interest is illuminated for between 1 seconds and 10 minutes, suitably between 5 seconds and 5 minutes, suitably between 10 seconds and 3 minutes, suitably between 30 seconds and 2 minutes. The time of illumination is dependent of the intensity of illumination. The skilled person will know how to adjust the time of illumination to achieve sufficient cleavage or alteration of the photocleavable groups.

In one embodiment, each location/area of interest is illuminated for 5 minutes. Suitably, therefore, step (c) comprises illuminating a location/area of interest for 5 minutes.

In one embodiment, each location/area of interest is illuminated for 30 seconds. Suitably, therefore, step (c) comprises illuminating a location/area of interest for 30 seconds.

Addition of Index Sequences

The methods of the invention comprise the addition of index sequences in order to form the spatial barcode attached to the or each root molecule, bridge molecule, or detection probe. Index sequences are added to a location or area that has been illuminated, and which therefore comprises root molecules, detection probes, bridge molecules or bound index sequences with exposed 5′ or 3′ ends. Suitably, exposed 5′ or 3′ ends are reactive.

Suitably an index sequence is added to any exposed, or reactive, 5′ or 3′ end present in the location or area illuminated in step (c). Suitably, in a first cycle of the methods, an index sequence is added to any exposed, or reactive, 5′ or 3′ end of a root molecule, bridge molecule, or detection probe present in the location or area illuminated in step (c). Suitably, in a subsequence cycle of the methods, an index sequence is added to any exposed, or reactive, 5′ or 3′ end of a bound index sequence present in the location or area illuminated in step (c).

Suitably the or each index sequence is added by ligation, which may be chemical or enzymatic. Suitably by ligation onto the 5′ or 3′ end of a root molecule, bridge molecule, or detection probe present in the location or area illuminated in step (c). Suitably in a first cycle of the methods. Suitably by ligation onto the 5′ or 3′ end of a bound index sequence present in the location or area illuminated in step (c). Suitably in a subsequent cycle of the methods. Suitably the or each index sequence is ligated by a ligase enzyme. Suitably the ligase enzyme may be selected from any ligase, such as: T4 ligase, T3 ligase, Taq ligase.

In one embodiment, the or each index sequence is ligated by T4 DNA ligase.

Suitably the or each bridge molecule is ligated to a detection probe by the same means.

Suitably, the ligase may be added to the methods of the invention during step (d) to ligate the or each index sequence. Suitably therefore step (d) may comprise ligating an index sequence of the spatial barcode to the or each root molecule or detection probe within the location or area illuminated in step (c).

Alternatively, the ligase may be added to the methods of the invention after step (e) to ligate all of the index sequences that have been added to the or each root molecule or detection probe. Suitably, in this embodiment, step (c) may comprise hybridising an index sequence of the spatial barcode to the or each root molecule or detection probe within the location or area illuminated in step (c). Suitably, the method further comprises a step after step (e) of ligating the index sequences to the or each root molecule or detection probe.

Index Sequence

The methods of the invention employ index sequences which when added together in various different orders form spatial barcodes. These spatial barcodes indicate where in a tissue sample a given detection probe was bound, and therefore where a relevant biological molecule or marker is expressed.

Suitably a spatial barcode is formed of a plurality of index sequences. Suitably, a spatial barcode comprises a plurality of index sequences. Suitably the index sequences are sequentially added together to form a spatial barcode, suitably by repeating steps (c) and (d) of the method. Suitably during each cycle of the methods, an index sequence is added to each root molecule, detection probe or bound index sequence. Suitably during the first cycle of the methods, a first index sequence is added to each root molecule, detection probe or bound index sequence, during a second cycle of the methods, a second index sequence is added to each root molecule, detection probe or bound index sequence and during subsequent cycles of the method, a third, fourth, etc. index sequence is added to each root molecule, detection probe or bound index sequence.

Suitably during the first cycle of the methods, a first index sequence is added to each detection probe or root molecule. Suitably during subsequent cycles of the methods, subsequent index sequences are added to each bound index sequence.

Each index sequence comprises:
a total length of between 5 and 50 nucleotides; and
a photocleavable group bound to one or both of the 5′ or 3′ ends of the molecule.

In one embodiment, the index sequences are nucleic acid sequences or nucleic acid mimics. Suitably comprising a 5′ and a 3′ end. Suitably, the index sequences may be RNA, DNA, or modified backbone nucleic acid sequences, comprised of canonical or non-canonical bases. In one embodiment, the index sequences are DNA. In one embodiment, each index sequence is a double stranded DNA. Suitably, each index sequence has a total length of between 10-40 nucleotides, suitably between 14-30 nucleotides, suitably between 15-25 nucleotides.

In one embodiment, each index sequence has a total length of 19-20 nucleotides.

Suitably the total length is the total length of the double stranded portion of the index sequence, suitably excluding any overhangs if present.

Suitably, each index sequence is produced by the annealing of nucleic acid strands having a total length of between 10-40 nucleotides, suitably between 14-30 nucleotides, suitably between 15-25 nucleotides.

In one embodiment, each index sequence is produced by the annealing of nucleic acid strands having a total length of 19-20 nucleotides.

Suitably each index sequence may comprise blunt ends.

Alternatively, each index sequence may comprise overhangs, suitably at both the 5′ and 3′ ends. Suitably the overhangs are complementary, suitably, the overhangs are complementary to overhangs on other index sequences. Suitably each overhang is partly or fully complementary to an overhang on another index sequence.

Suitably each overhang comprises a length of between 1-15 nucleotides, suitably 3-9 nucleotides. Suitably each overhang comprises a length selected from 3, 4, 5, 6, 7, 8 and 9 nucleotides. Suitably each overhang is 6 or 7 nucleotides in length.

Suitably each index sequence comprises a first overhang and a second overhang. Suitably the first and second overhangs may be independently located at the 5′ or 3′ ends of each index sequence.

In one embodiment, the overhangs located at the 5′ and 3′ end of the or each index sequence have the same length.

In one embodiment, the overhangs located at the 5′ and 3′ end of the or each index sequence have different lengths. Suitably each index sequence comprises a longer and a shorter overhang, located at either end of the molecule. Suitably a first longer overhang and a second shorter overhang. Suitably a longer overhang is located at a first end of the index sequence and a shorter overhang is located at a second end of the index sequence.

Suitably each index sequence comprises a first overhang of 6 nucleotides in length and a second overhang of 7 nucleotides in length. Suitably when the index sequences are added together to form a spatial barcode, the overhangs of the index sequences alternate. Suitably the overhangs alternate between 6 nucleotides in length and 7 nucleotides in length.

Suitably each index sequence comprises one or more photocleavable groups. The or each photocleavable group is as defined elsewhere herein.

Suitably, each index sequence comprises a central region having a unique nucleotide sequence distinct from that of all other index molecules.

Suitably each index sequence comprises a high GC content. Suitably each index sequence comprises a GC content of between 30% and 80%.

Suitably, each index sequence does not form any AA or TT dimers. Suitably when an index sequence is a double stranded DNA, it does not comprise any AA or TT dimers.

The present invention further provides a library of index sequences.

Suitably the library of index sequences comprises index sequences to be used in the methods of the invention. Suitably the library of index sequences comprises all of the index sequences to be used in the methods of the invention.

Suitably there are at least 4 different index sequences used in the method of the present invention. Suitably between 1-100 different index sequences may be used in the methods of the present invention. In one embodiment, 4 different index sequences are used in the present invention. Suitably a higher number of index sequences allows longer spatial barcodes to be generated, and therefore a higher number of unique barcodes to be generated, and therefore more locations/areas of interest to be labelled.

Suitably the index sequences may be classified into groups. Suitably the index sequences in each group have the same nucleotide sequence. Suitably the library may comprise a plurality of groups of index sequences.

Suitably therefore the library may comprise a plurality of index sequences, suitably a plurality of groups of index sequences. Suitably the library may comprise at least 2 groups of index sequences, wherein the index sequences in each group share the same nucleotide sequence. Suitably the library may comprise up to 100 groups of index sequences, wherein the index sequences in each group share the same nucleotide sequence.

For example, the library of the invention may comprise 4 groups of index sequences; group A, group B, group C, group D, wherein the index sequences in each group share the same nucleotide sequence.

In one embodiment, an index sequence may comprise a sequence according to any of SEQ ID NO: 17-25, 27-30, 33-36 and 38-77. In one embodiment, an index sequence may comprise a pair of sequences selected from any of SEQ ID NO: 17-25, 27-30, 33-36 and 38-77, suitably wherein the pair of sequence are capable of annealing to each other.

In one embodiment, the library of index sequences may comprise any of SEQ ID NO: 17-25, 27-30, 33-36 and 38-77. In one embodiment, the library of index sequences may comprise a plurality of any of SEQ ID NO: 17-25, 27-30, 33-36 and 38-77. In one embodiment, the library of index sequences may comprise any pair of sequences selected from SEQ ID NO: 17-25, 27-30, 33-36 and 38-77, suitably wherein the pair of sequence are capable of annealing to each other. In one embodiment, the library of index sequences may comprise a plurality of pairs of sequences selected from SEQ ID NO: 17-25, 27-30, 33-36 and 38-77, suitably wherein each pair of sequence are capable of annealing to each other.

Suitable pairs of sequences within SEQ ID NO: 17-25, 27-30, 33-36 and 38-77 which may anneal to form an index sequence are identified in the examples herein. Any such pair forming an index sequence is an embodiment of the invention.

Spatial Barcode

The present invention provides methods of spatial barcoding. These methods comprise the addition of a spatial barcode to root nucleic acid molecules or detection probes, optionally through a bridge molecule, in order to label where each root molecule or detection probe is bound.

The invention further provides a spatial barcode comprising a plurality of index sequences, wherein the index sequences are selected from the library as defined elsewhere herein.

As described above, each spatial barcode is formed of a plurality of index sequences. Suitably the index sequences in each spatial barcode are arranged in a unique order. Suitably, therefore, each spatial barcode is unique.

Suitably, the individual index sequences forming a spatial barcode are linked by a covalent chemical bond. Suitably the covalent chemical bond is compatible with polymerase enzymes, and compatible with high-throughput sequencing chemistry. Suitably the covalent chemical bond is compatible with polymerase enzymes.

Suitably, the individual index sequences forming a spatial barcode are linked by a phosphodiester bond.

Suitably one spatial barcode is allocated per each location or area of interest. Suitably a spatial barcode is unique to a selected location or area. Suitably, the same spatial barcode is added to each root molecule, bridge molecule or detection probe within the same location or area of interest. Suitably therefore each spatial barcode indicates a given location or area of interest.

Suitably the or each spatial barcode comprises at least one index sequence. Suitably the or each spatial barcode comprises between 4-50 index sequences. Spatial barcodes comprising a higher number of index sequences have a higher encoding capacity and can label more unique locations/areas of interest. Suitably each index sequence within a spatial barcode may be the same or different.

Suitably the index sequences are added to the or each root molecule, bridge molecule or detection probe in a specific order to build up the spatial barcode. Suitably one index sequence is added to the or each root molecule, bridge molecule or detection probe in a first cycle of steps (c) and (d). Suitably one index sequence is then added to the or each detection probe per subsequent cycle of steps (c) and (d). Suitably by adding to the bound index sequences. Suitably steps (c) and (d) are repeated in cycles until the spatial barcode is fully formed and attached to the or each detection probe. Suitably therefore, the number of cycles of steps (c) and (d) is determined by the length of the or each spatial barcode.

Suitably, the order of index sequences in each spatial barcode is optimised to reduce errors during sequencing.

The present invention further provides a library of spatial barcodes.

Suitably, each spatial barcode in the library comprises a plurality of index sequences, wherein the index sequences are selected from the library of index sequences as defined elsewhere herein. Suitably, each spatial barcode in the library is unique. Suitably, each spatial barcode in the library comprises a unique combination of index sequences.

Suitably, the library of spatial barcodes may be designed in order to reduce mis-identification errors after sequencing. Suitably, the library of spatial barcodes forms an error-correcting code. Many methods of producing error-correcting codes are known in the art

Suitably, the combination of index sequences in each spatial barcode included in the library may be chosen so that each spatial barcode has a Hamming distance of 1 from all other spatial barcodes included in the library. Suitably each spatial barcode has a Hamming distance of 1 from all other spatial barcodes used in a method of the invention.

Suitably, the Hamming distance between a pair of spatial barcodes is defined as the number of elements (in this case index sequences) in the first spatial barcode that have to be replaced with other index sequences in order to transform the first spatial barcode into a copy of the second spatial barcode.

Suitably, the combination of index sequences in each spatial barcode included in the library of spatial barcodes may be chosen so that each spatial barcode has a Hamming distance of 3, 5, or 7 from all other spatial barcodes included in the library. Suitably each spatial barcode has a Hamming distance of 3, 5, or 7 from all other spatial barcodes used in a method of the invention.

Suitably, the combination of index sequences in each spatial barcode included in the library of spatial barcodes may be chosen according to an error-correcting encoding scheme capable of correcting at least one, at least two or at least three substitution, deletion or insertion errors. Suitably the methods of the invention may comprise a step of assigning a spatial barcode to each location or area of interest within the tissue. Suitably this step occurs prior to step (c). Suitably assigning a spatial barcode to each location or area of interest is carried out using software. Suitably assigning a spatial barcode to each location or area of interest is automatically carried out by software, suitably when a location or area of interest is selected by a user.

Suitably an assigned spatial barcode comprises a plurality of units. Suitably each unit corresponds to an index sequence. Suitable units may be any form of code, for example numbers or letters wherein each index sequence has a corresponding unit. For example, in an embodiment where 4 different index sequences are being used and the spatial barcode has a length of 4 units, units A, B, C and D may each correspond to a different index sequence. In such an embodiment, examples of assigned spatial barcodes may be: ABCD, ACBD, ADBC and the like.

Sequencing

After the complete spatial barcodes are added to the root nucleic acid molecules, bridge molecules or detection probes, a step of sequencing may then take place.

Suitably, sequencing may not take place immediately after the spatial barcodes are added. In some embodiments, the substrate or tissue comprising the spatial barcodes attached to root molecules, bridge molecules, or detection probes may be stored prior to sequencing. The present invention therefore provides a tissue comprising spatially barcoded detection probes. The present invention further provides a substrate comprising spatially barcoded root molecules.

Suitably, when the complete spatial barcode has been added to a detection probe, the detection probe is then known as a spatially barcoded detection probe. Similarly, when a complete spatial barcode has been added to a root molecule, the root molecule is then known as a spatially barcoded root molecule.

Suitably the or each spatially barcoded root molecule or detection probe is sequenced. Suitably therefore, the or each root molecule or detection probe and the attached spatial barcode are sequenced as a single nucleic acid, optionally further comprising a bridge molecule.

Suitably the detection probes provide information on what biological molecules are expressed and to what level in the tissue.

Suitably the spatial barcodes provide information on where the biological molecules are expressed in the tissue. Suitably, in which areas of interest the biological molecules are expressed.

Suitably therefore, in sequencing a single nucleic acid produced by the methods of the invention, identification, quantification and spatial information is provided for each biological molecule of interest.

Suitably the methods of the invention may further comprise a step of preparing the one or more spatially barcoded detection probes or root molecules for sequencing. Suitably this step occurs prior to the sequencing step.

In one embodiment, this includes removing the spatially barcoded detection probes or root molecules from the substrate or tissue. In another embodiment, a portion or all of the spatially barcoded detection probes or root molecules are amplified in situ, prior to preparation for sequencing.

Suitably, preparing the one or more spatially barcoded detection probes or root molecules for sequencing may comprise adding modifiers to the or each spatially barcoded detection probe or root molecule. Suitable modifiers may be those required to conduct sequencing, for example a primer or a PCR handle.

Suitably, a sequencing element, such as a sequencing primer required for sequencing library preparation, may be added to the end of each spatial barcode. Suitably to the 5′ end, or suitably to the 3′ end. Suitably the sequencing elements are added by PCR, enzymatic ligation, or by template switching of reverse transcription. Suitably in the case of adding to the 5′ end, the sequencing elements are added by ligation. Suitably in the case of adding to the 3′ end, the sequencing elements are added by template switching of reverse transcription, or by PCR or by ligation. Suitably addition of sequencing elements by PCR may comprise using random hexamer oligonucleotides comprising the sequence element at the 5′ end thereof. Suitably addition of sequencing elements by ligation may comprise a step of fragmentation of an elongated detection probe, suitably prior to ligation. Suitably using any ligase enzyme known in the art, or by using any reverse transcriptase known in the art, or by using any DNA polymerase enzyme known in the art. Suitably, the ligase enzyme used may one of the ligase enzymes described elsewhere herein. Suitably, the addition of a sequencing element is performed before step (f) of the methods of this invention. Alternatively, if the sequencing element is a 3′ primer, then the addition of the sequencing element can be performed at the same time as the detection probe is elongated, suitably between steps (a) and (b) of the methods of the invention. Suitably between steps (a) and (b) of a method of the third aspect which may comprise an elongation step as described hereinabove.

Suitably, one or more spatially barcoded detection probes or root molecules may be extracted from the tissue or specimen by any DNA extraction method known in the art, and the resulting pool of molecules may be stored prior to sequencing.

Suitably, preparing the one or more spatially barcoded detection probes or root molecules for sequencing may comprise a step of transcription. Suitably transcribing the or each spatially barcoded detection probe or root molecule into RNA.

Suitably, preparing the one or more spatially barcoded detection probes or root molecules for sequencing may comprise a step of isolating the or each spatially barcoded detection probe or root molecule. Suitably a step of isolating the or each spatially barcoded detection probe RNA.

Suitably, preparing the one or more spatially barcoded detection probes or root molecules for sequencing may comprise a step of reverse transcription. Suitably reverse transcribing the or each spatially barcoded detection probe RNA.

Suitably, preparing the one or more spatially barcoded detection probes or root molecules for sequencing may comprise a step of amplifying the one or more spatially barcoded detection probes or root molecules. Suitably, amplifying the or each reverse transcribed spatially barcoded detection probe.

Suitably, the one or more spatially barcoded detection probes or root molecules may be amplified by an enzymatic process using the amplification region included in each detection probe or root molecule. Suitably, this amplification can happen while the spatially barcoded detection probes or root molecules are still embedded in the tissue, or after they have been extracted as described above. In one embodiment, the amplification is performed by RNA transcription, in one embodiment, the enzyme used for amplification is T7 RNA polymerase.

Alternatively, the amplification may be carried out by any other known amplification processes, for example rolling circle amplification. Suitably in such embodiments, the spatially barcoded detection probe is first circularised, suitably by a telomerase enzyme, suitably teIN polymerase. Suitably the circularised spatially barcoded detection probe is then amplified, suitably by a strand-displacement polymerase, suitably by Phi29 DNA polymerase.

Suitably, the amplification process produces multiple copies of each spatially barcoded detection probe or root molecule, replicating the sequence of the detection probe or root molecule and of the spatial barcode. In one embodiment, such copies are RNA molecules.

Suitably therefore, preparing the one or more spatially barcoded detection probes or root molecules for sequencing may comprise: adding modifiers to the or each spatially barcoded detection probe or root molecule, transcribing the or each spatially barcoded detection probe or root molecule into RNA, isolating the or each spatially barcoded detection probe or root molecule RNA, reverse transcribing the or each spatially barcoded detection probe or root molecule RNA into DNA, and amplifying the or each reverse transcribed spatially barcoded detection probe or spatially barcoded root molecule DNA.

Suitably after reverse transcription, the spatially barcoded detection probes or spatially barcoded root molecules form a sequencing library ready for sequencing.

System

The present invention further provides an integrated system to perform the methods of spatial barcoding described herein.

The integrated system comprises:
An instrument for viewing a substrate;
a light source for illuminating one or more locations of the substrate;
a microfluidic circuit for delivering one or more index sequences and reagents to the substrate;
a processor for implementing software operable to control the microscope, light source, and microfluidic circuit.

In one embodiment, the substrate is a tissue, as described above. In one embodiment, the system is for spatially barcoding one or more detection probes, and/or one or more root molecules, and/or spatially barcoding one or more markers. Suitably in one or more areas of interest. Suitably in one or more areas of interest of a tissue sample. Suitably the instrument is for viewing a substrate. Suitably the instrument is for viewing a tissue sample. Suitable tissue samples are described elsewhere herein. Suitably, the instrument is further used for directing the illumination, suitably for directing illumination from the light source, suitably onto the substrate.

In one embodiment, the instrument may be a microscope.

Suitably, the microscope is a light microscope. Suitably, the microscope may have a low magnification. Suitably, the microscope may have a diffraction limited resolution or above. Suitably the microscope may have a resolution of 200 nm or above. Suitably, the microscope may have a resolution of 300 nm or above.

Suitably, the microscope design can be any design known in the art, including commercial instruments, as long as this can work in conjunction with the light source, illumination path and fluidic system described herein.

Suitably the microscope may comprise an objective compatible with the illumination to be used, suitably with infrared, visible, or UV light. Suitably, the microscope is compatible with wavelengths of light that are to be used for photocleaving as described elsewhere herein.

Suitably, the microscope system may include a motorized stage controlled by software. Suitably, the microscope can include a motorized focusing turret controlled by software. Suitably, the microscope can include an automated closed-loop focusing system to track the substrate to be processed by the methods of this invention.

Suitably, the microscope may comprise the light source.

Suitably, the light source can produce illumination as described elsewhere herein (in the “illumination” section). Suitably, the light source is a lamp, laser or a LED. In one embodiment, the laser may be a high-power laser.

Suitably, the illumination may be directed to each location or area of interest. Suitably by using a refractive or reflective optical system. Suitably the refractive or reflective optical system may have a diffraction limited resolution or above. Suitably the microscope may have a resolution of 200 nm or above. Suitably the optical system may be comprised within a microscope, such as any microscope described in the art. Suitably, the optical system includes an element to direct illumination to the or each location or area of interest. Suitably, the optical system includes an element to direct illumination from the light source to the or each location or area of interest.

In some embodiments, the element is a movable mirror, for example a galvanometric mirror. In some embodiments, the element is a digital micromirror device (DMD chip). In some embodiments, the element is a spatial light modulator.

Suitably, the optical system may further comprise elements such as a beam expander, alignment mirrors, and light intensity regulators.

Suitably the processor implements software which is operable to:
(i) conduct image processing of the tissue;
(ii) assign a spatial barcode to each selected location or area of interest of the substrate or tissue;
(iii) control illumination of the selected locations or areas of interest; and/or
(iv) control fluid flow through the microfluidic circuit.

Suitably the processor implements software which is operable to carry out all functions of the system. Suitably the processor implements software which is operable to carry out each of functions (i) to (iv).

Suitably the software is operable to conduct image processing of images of the tissue. Suitably the images of the tissue are obtained using the microscope and a camera. Suitably image processing may comprise one or more of the following steps: Pre-processing, Local thresholding, Pixel classification, Watershed segmentation and Object classification. Suitably image processing of the images allows a user to more easily select one or more areas of interest from an image, especially when the image is of tissue. Suitably image processing of the images allows a user to more easily select one or more areas of interest from an image, such as biological features, collections of cells, individual cells, or subcellular compartments.

Suitably the microfluidic circuit transports fluids through the system. Suitably the microfluidic circuit transports reagents and index sequences through the system. Suitably microfluidic circuit transports reagents and index sequences through the system to contact the tissue. Suitably therefore the microfluidic circuit is for delivering reagents and index sequences to the tissue.

Suitably the microfluidic circuit may comprise channels. Suitably the channels deliver index sequences and reagents to the tissue. Suitably the channels are in fluid communication with the tissue.

Suitably the microfluidic circuit comprises storage chambers. Suitably the storage chambers are for storing the index sequences and reagents. Suitably the microfluidic circuit further comprises channels connecting the storage chambers to the tissue. Suitably the channels are in fluid communication with the storage chambers.

Suitably the microfluidic circuit may further comprise a flow cell. Suitably the flow cell comprises the tissue. Suitably the flow cell may comprise a mount or stage for the tissue, suitably the stage may be motorised as described above. Suitably the flow cell may be located within the field of view of the microscope. Suitably the channels are in fluid communication with the flow cell. Suitably the channels are in fluid communication with the flow cell and with the storage chambers.

Suitably reagents and/or index sequences can flow from the storage chambers to the tissue/substrate via the channels of the microfluidic circuit.

Suitably the microfluidic circuit may further comprise an outlet to allow waste reagents and index sequences to exit the system. Suitably the microfluidic circuit may further comprise valves to control the movement of fluid through the circuit. Suitably the valves and outlet may also be controlled by the processor.

Further features and embodiments of the present invention will now be described by reference to the following figures in which:

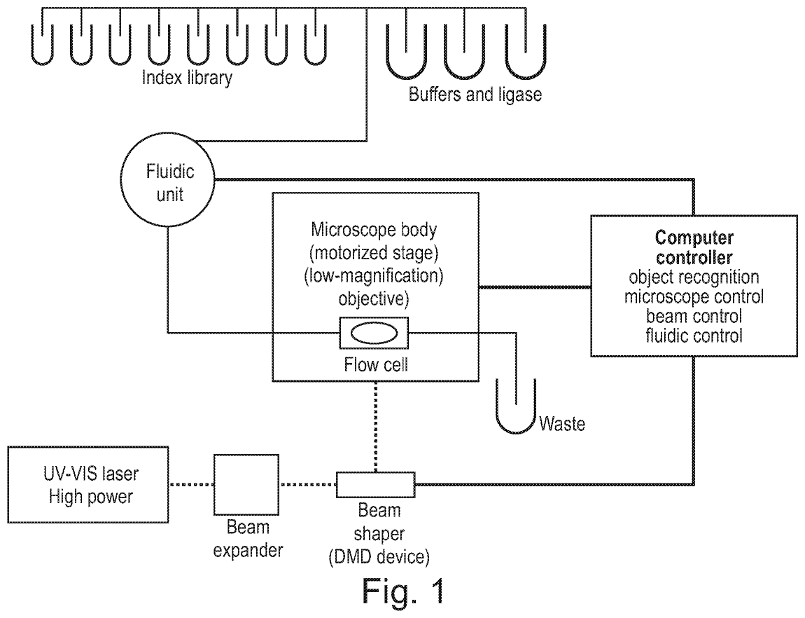

FIG. 1 shows: A schematic of an embodiment of the system of the invention. The system includes a microscope body, which can include a low-magnification objective, motorized stage, and motorized focus; a fluidic circuit connected to several reservoirs for index molecules, reagents, buffers and ligase enzymes, a flow cell in which the specimen is placed, a light source comprising a high-power LED or laser in the UV, visible or IR spectrum, an optical path used to direct light from the light source into the microscope, and a beam shaper used to produce patterned illumination (i.e. a digital micro-mirror device). All of the components of the system are controlled by a computer implementing an integrated control software;

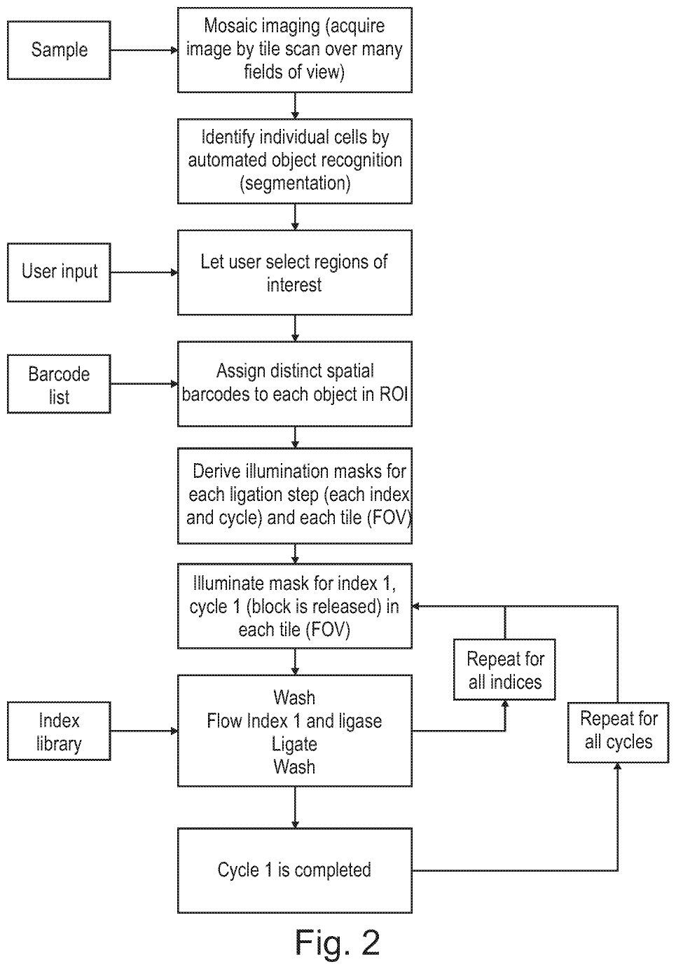

FIG. 2 shows: A flow-chart of an embodiment of the method of the invention. The process includes imaging of the specimen, identification of individual areas of interest by automated segmentation, selection of a subset of the areas of interest for spatial barcoding, assignment of individual spatial barcodes to each area of interest, and a cyclical spatial barcoding process in which 1) illumination is applied to some of the areas of interest to release a photocleavable group, making the DNA molecules there contained compatible with extension by ligase, 2) flowing an index over the sample, 3) using an enzymatic process to link the enzyme to the DNA molecules in the area(s) of interest that were illuminated, 4) wash and repeat;

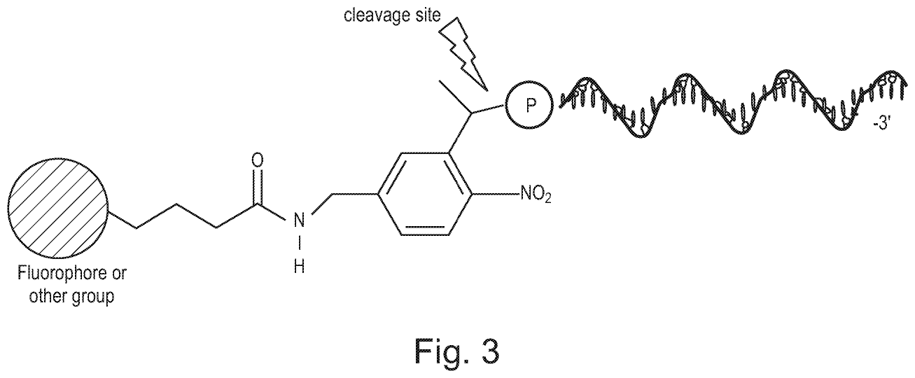

FIG. 3 shows: The structure of an embodiment of a photocleavable 5′ block on an oligonucleotide. The photocleavable group is released by illumination in the UV or violet range (340 nm to 410 nm) and yields an accessible 5′ phosphate group on the oligonucleotide, which can be targeted by ligation. The photocleavable group can be linked, on the side opposite to the protected phosphate, to a fluorophore group or to another nucleotide. This allows detection of the 5′ block and its release via fluorescence microscopy;

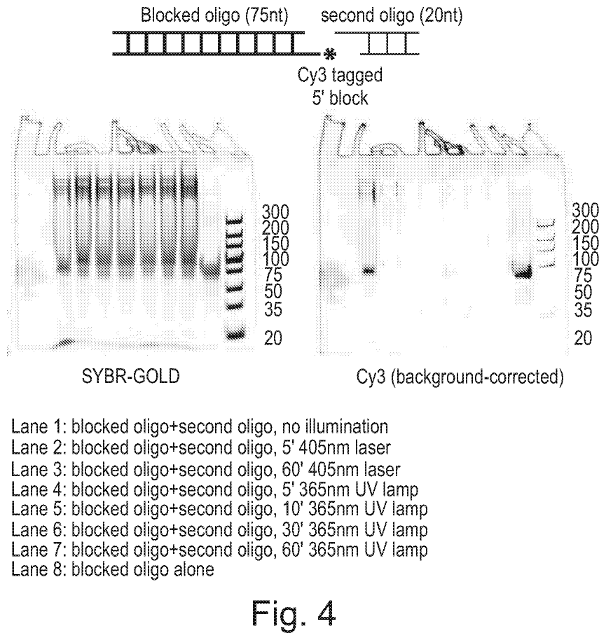

FIG. 4 shows: Proof of concept of light-triggered ligation. A 75 bp duplex oligonucleotide with a photo-cleaved 5′ end (in which the photocleavable group was tagged with a fluorophore) was irradiated with light of different intensities and wavelength and incubated with a second shorter oligonucleotide (20 bp) and T4 ligase. In all the irradiated samples, the photocage was completely removed (see loss of cy3 fluorescence) and the two oligos were ligated, producing a novel species at 95 bp. The high molecular weight bands in lanes 1-7 correspond to the two oligonucleotides still bound to the ligase enzyme (which affects their electrophoretic mobility);

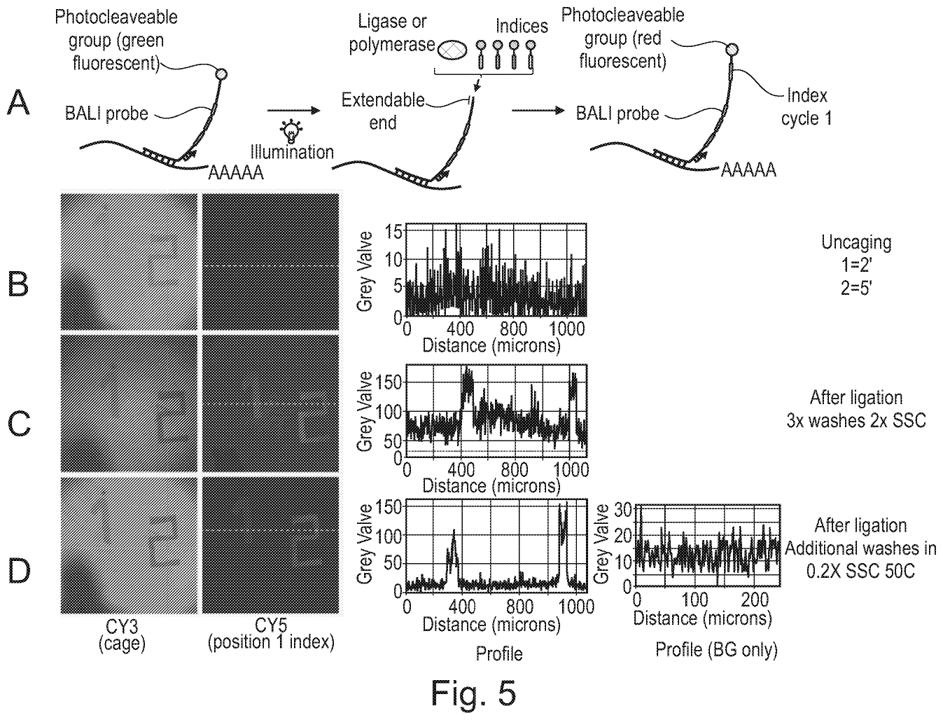

FIG. 5 shows: Proof of concept for the attachment of an index molecule to a detection probe attached to a solid surface (glass slide). The schematics (A) indicate the process happening during the experiment. The photocleavable group at the 5′ of a detection probe (BALI probe) is cleaved by illumination, and an index is ligated. In this case, the photocleavable group is labelled with the cy3 fluorescent dye, and the index with the cy5 fluorescent dye. The image on the bottom indicates: (B) first row: fluorescence image of the specimen surface after illumination has been applied to cleave the photocleavable group in two areas of interest with the shape of a “1” and “2” numbers. The “1” area was irradiated for 2 minutes, and the “2” area for 5 minutes. The Cy3 signal is decreased in the irradiated areas in a way proportional to irradiation time, indicating removal of the photocleavable group. There is no cy5 signal. (C) fluorescence image after ligation of a cy5 labelled index and initial washing of unligated indices. The irradiated areas are now positive for cy5 signal, while the non-irradiated areas show some background signal indicating a certain amount of indices bound a specifically. (D) fluorescence image of the same sample after extended washes. The cy5 signal in the non-irradiated areas is now back to the values that it had prior to the ligation, indicating removal of the index from all areas except the irradiated ones, where the index has been added to the growing spatial barcode. The plots on the right of each image correspond to the intensity profile of the cy5 image over the dotted line;

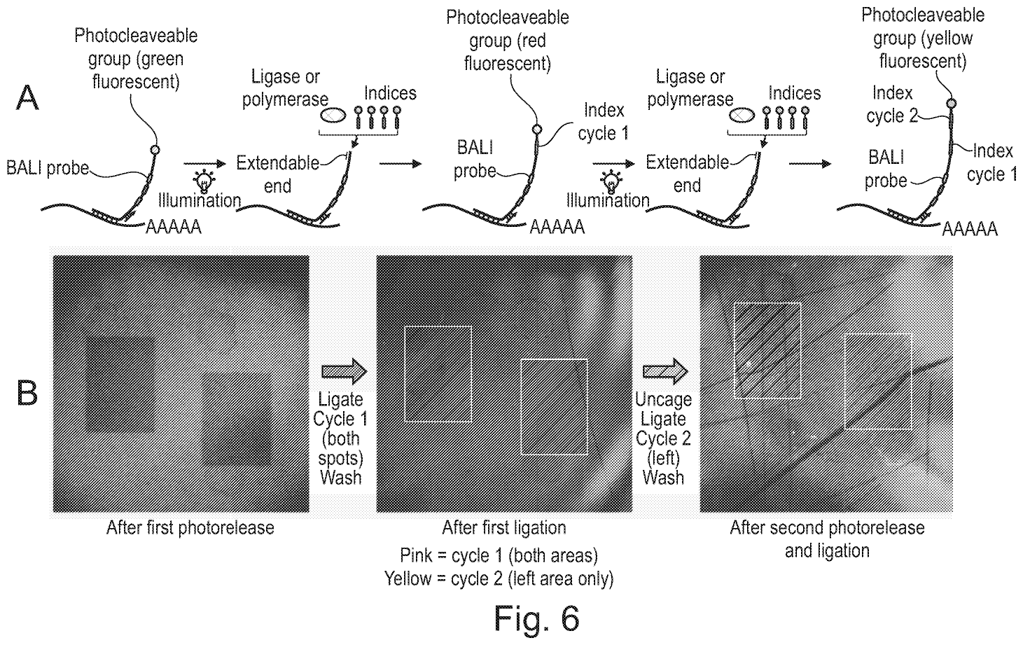

FIG. 6 shows: Proof of concept of spatial barcoding on a solid surface with two cycles of index ligation plus DNA bridge ligation. The schematics on top (A) indicate the process happening during the experiment. The detection probe (BALI probe) is ligated to a DNA bridge molecule bearing a photocleavable group labelled with the Alexa 488 fluorophore (cyan). The photocleavable group is cleaved by illumination, and a first index is ligated which bears a second photocleavable group labelled with the cy5 fluorophore (violet). The second photocleavable group is again removed by illumination, and a third index, labelled with Atto 568, is ligated (yellow). The images on the bottom (B) show the slide surface after the first photorelease step, after the ligation of the first index, and after the ligation of the second index. Two areas with reduced Alexa 488 signal (cyan) are visible on the left image, corresponding to the areas that have lost the first photocleavable group on the DNA bridge molecule. In the middle image, both areas show cy5 signal (pink), indicating successful ligation of the first index. In the right image, the leftmost area shows a reduced cy5 signal, due to the removal of the second photocleavable group, and an Atto568 signal (yellow), indicating successful ligation of the second index;

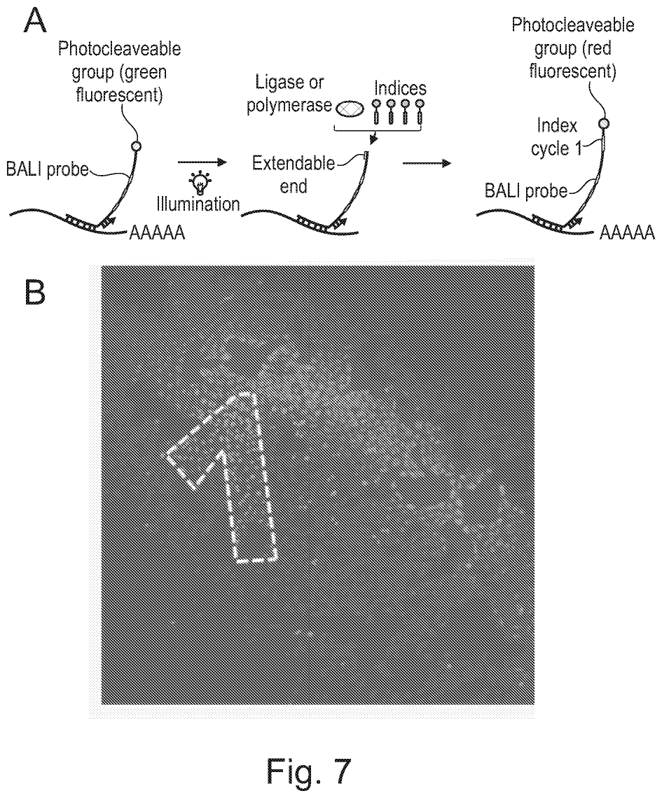

FIG. 7 shows: Proof of concept of light control of index ligation on cells. The schematic on top (A) indicates the process happening during the experiment. The photocleavable group at the 5′ of a detection probe (BALI probe) is cleaved by illumination, and an index is ligated. In this case, the photocleavable group is labelled with the cy3 fluorescent dye, and the index with the cy5 fluorescent dye. The image below (B) corresponds to the cy5 channel (in red) image collected on the fluorescence microscope after the ligation of the tagged index. The shape of the area of interest (a “1”) can be seen as an area of increased cy5 signal. In the top left of the image a red halo is visible, corresponding to a reflection of the 647 nm laser on the surface of the coverslip (not real signal);

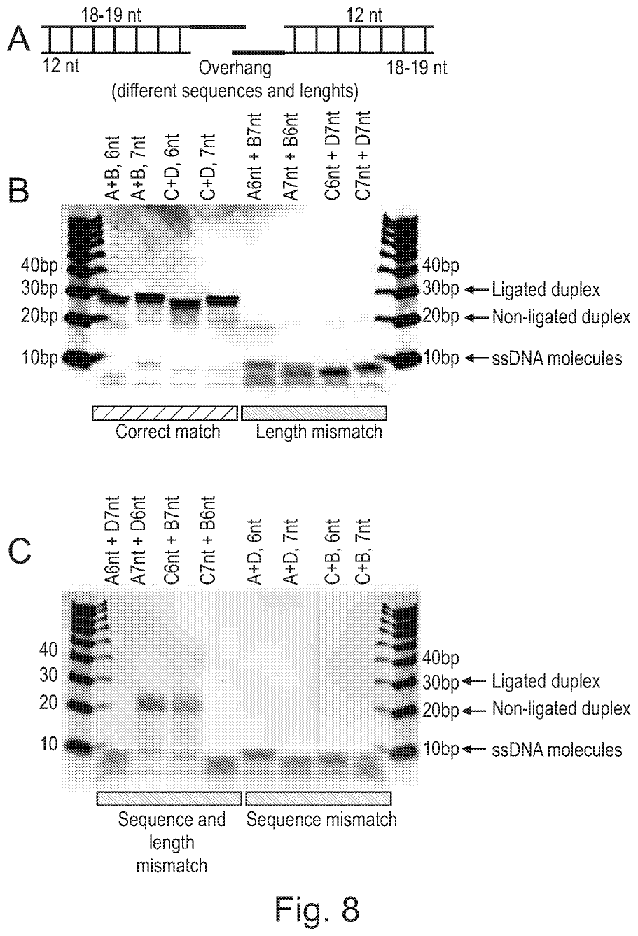

FIG. 8 shows: Index sequence optimization by comparison of different index overhang sequences and length for the purpose of maximizing ligation efficiency and minimizing cross-talk between barcodes. The schematic on top (A) describes the process happening during the experiment: a first DNA duplex (corresponding to the last portion of a detection probe, bridge DNA molecule, or root DNA molecule, or to an index) is ligated to a second DNA duplex representing an index sequence. Each duplex is produced by the annealing of a 12 nt and a 18-19 nt DNA oligonucleotides. The 5′ overhang of both molecules is different between different lanes in the experiment. There are four overhang sequences, named A-D. The overhang of sequence A is complementary to the overhang of sequence B, and the overhang of sequence C is complementary to the overhang of sequence D. Furthermore, each overhang can have two different lengths, 6 nt or 7 nt. DNA duplexes containing different combinations of the overhangs are mixed and ligated by T4 ligase. The images on the bottom correspond to a non-denaturing agarose gel in which the molecular species produced by the ligation reaction. The lanes are loaded as follows: GEL 1 (B): 1: DNA length ladder, 2: overhang A+B, both 6 nt, 3: overhang A+B, both 7 nt, 4: overhang C+D, both 6 nt, 5: overhang C+D, both 7 nt, 6: overhang A6 nt+B7 nt, 7: overhang A7 nt+B6 nt, 8: overhang Clint+D7 nt, 9: overhang C7 nt+D6 nt, 10: DNA length ladder, GEL 2 (C): 1: DNA length ladder, 2: overhang A6 nt+D7 nt, 3: overhang A7 nt+D6 nt, 4: overhang C6 nt+B7 nt, 5: overhang C7 nt+B6 nt, 6: overhang A+D, both 6 nt, 7: overhang A+D, both 7 nt, 8: overhang C+B, both 6 nt, 9: overhang C+B, both 7 nt, 10: DNA length ladder. Bands are visible at ˜30 bp (ligated duplex, 30 or 31 bp), ˜20 bp (non-ligated duplex, 19 or 20 bp with overhangs), and below 10 nt (single stranded DNA species, running lower than the corresponding dsDNA band). The non-ligated duplex band is sometimes absent as the conditions under which the gel was ran (in particular temperature) could cause its denaturation in some cases. A band corresponding to the ligated duplex is only present when both the length and the sequence of the overhangs are compatible, indicating no cross-talk between different overhangs. The ligation efficiency is in the range of 80%/90%;

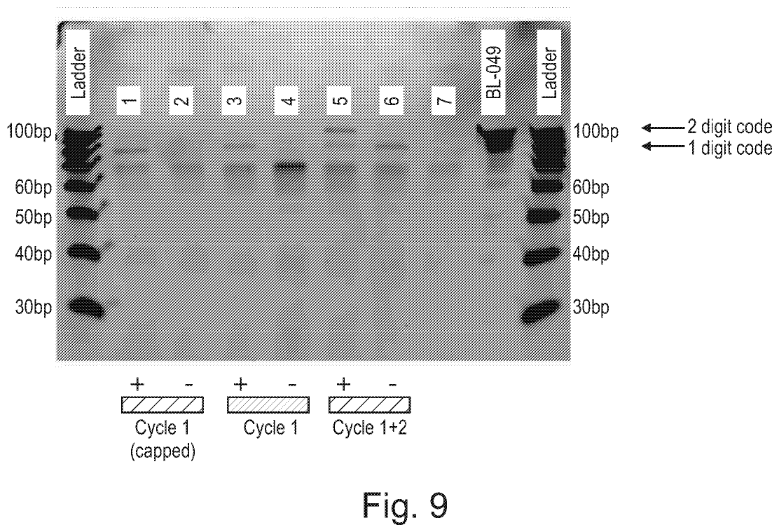

FIG. 9 shows: Proof of concept of the production of a spatial barcode by sequential ligation of index molecules on a detection probe bound to a solid surface (magnetic bead), using the optimized index molecule design tested in example 5. Two cycles using indices without photocleavable groups. The image represents a denaturing TBE-urea poly-acrylamide gel on which different samples are loaded according to Table 6. For samples 1-7, the material loaded on the gel is the RNA produced from T7 transcription of the detection probe linked to the spatial barcode. The detection prove length is 100 nt, and the RNA produced by the detection probe alone is 70 nt. Each index adds approx. 20 nt to the molecule. Therefore, the expected RNA length for the non-extended detection probe is 70 nt, for the detection probe+1 index is 90 nt, and for the detection probe+2 indices is 110 nt. The 100 nt BALI_26 oligo is loaded in the second to last lane for size reference. The first and last lane are loaded with DNA size ladder.

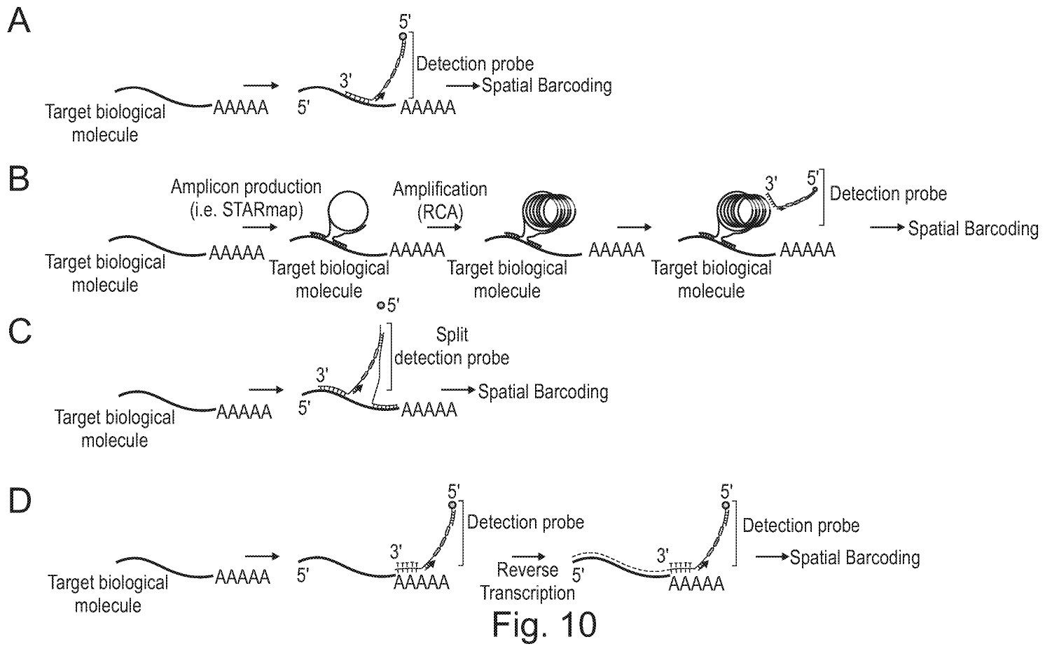

FIG. 10 shows: schematic diagrams of various options encompassed within the methods of spatial barcoding of the invention relating to the detection probe; (A) typical spatial barcoding process showing a detection probe bound to a nucleic acid of interest, where the detection probe comprises a binding region at the 3′ end, a species barcode, and a photocleavable group at the 5′ end; (B) a spatial barcoding process which further comprises a pre-amplification step in which an amplification product is produced by rolling circle amplification from the nucleic acid of interest, prior to binding of the detection probe; (C) a spatial barcoding process which uses a split detection probe comprising a first part and a second part which both bind to a nucleic acid of interest and anneal to each other to form a whole detection probe; (D) a spatial barcoding process in which the detection probe binds to the polyA region of a nucleic acid of interest, typically for transcriptome analysis, which includes a step of elongating the detection probe by reverse transcription at the 3′ end to form a detection probe comprising a sequence complementary to the transcript of interest.

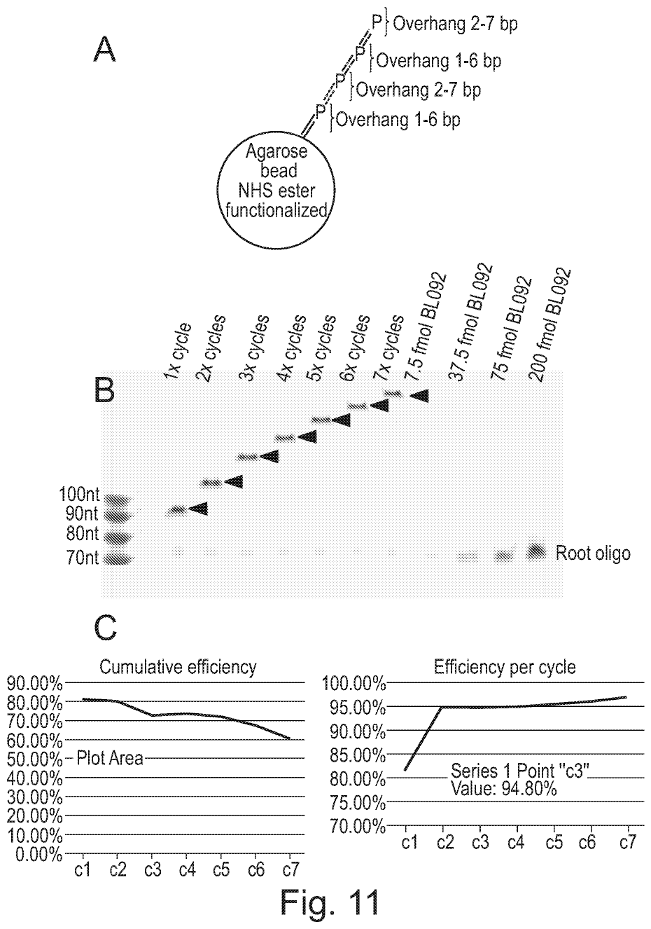

FIG. 11 shows: results of an experiment performing cyclic barcoding on gel beads to produce a spatial index of length from 1 to 7 bits (example 7). (A) scheme of the experiment: a double strand DNA root molecule modified with a fluorescent group is attached to an agarose bead and extended by several cycles of ligation using different index sequences and two alternating ligation overhangs. (B) results of experiment detected by denaturing poly-acrylamide gel electrophoresis. Lane 1 is a DNA length marker, lane 2 is the molecule after 1 cycle of ligation, lane 3-8 are the molecule after 2-7 cycles of ligation, lane 9-12 are increasing concentrations of the root molecule alone used to quantify molecular abundance by densitometry (C) left: estimate of the cumulative ligation efficiency after 1-7 cycles, defined as the fraction of fully extended root molecule over the total root molecule present in the experiment. Right: estimate of the per-cycle ligation efficiency for each of the ligation steps, defined as the fraction of extended root molecule for that cycle over the total amount of root molecule elongated to at least the step immediately before the one being measured. The first cycle has lower efficiency, presumably due to steric hindrance of the gel bead.

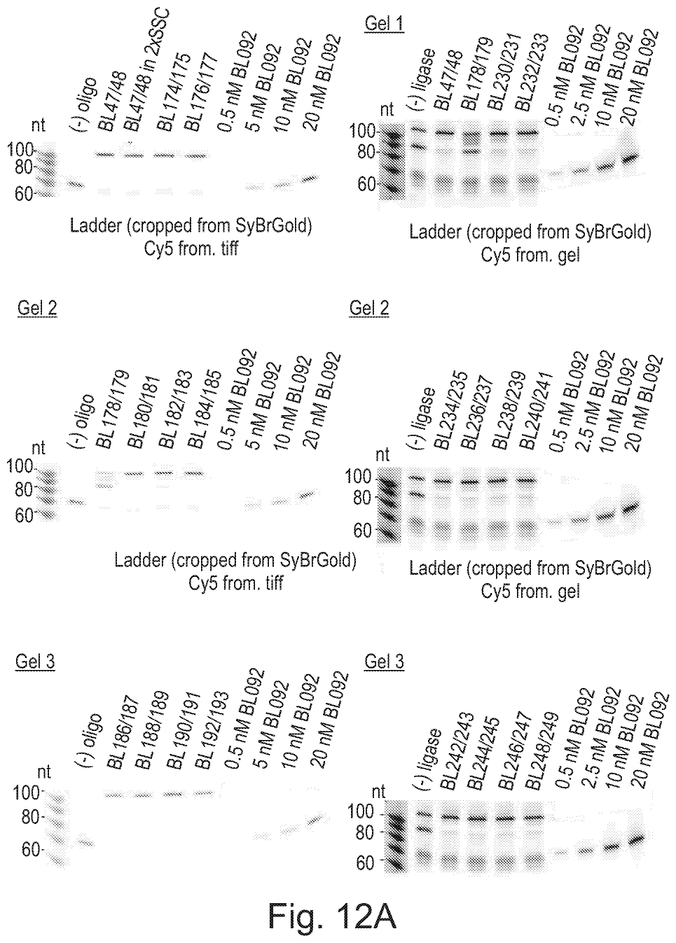

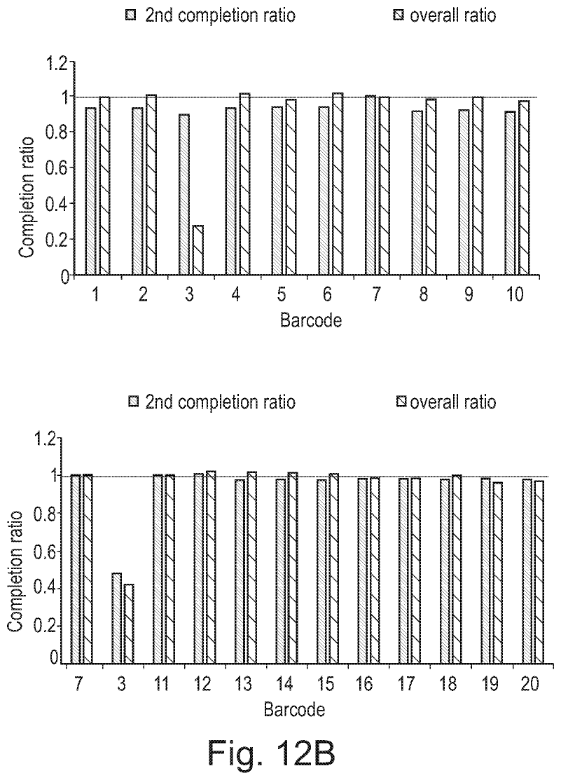

FIG. 12 shows: results of an experiment (example 8) comparing different index sequences for their ligation efficiency in conditions similar to the ones described in example 7. Specifically, the different sequences were tested for ligation in position 2 of an elongating spatial barcode, in order to avoid the reduced efficiency due to the steric hindrance of the gel bead shown in FIG. 11. (A) poly-acrylamide gel electrophoresis showing the root molecule (approx. 70 nucleotides) and the ligation product with each of the index sequence (over 100 nucleotides). Sequence IDs are described in example 8. Each gel also includes several lanes loaded with increasing concentration of the root oligo used to calibrate densitometry quantification. (B) estimate of the ligation efficiency for each index sequence, normalised by the efficiency of index sequence 7 (barcode 7 in example 8), chose as reference. The efficiency was calculated as the ratio between the abundance of the elongated root molecule (over 100 nucleotides) over the total abundance of the root molecule used in the experiment.

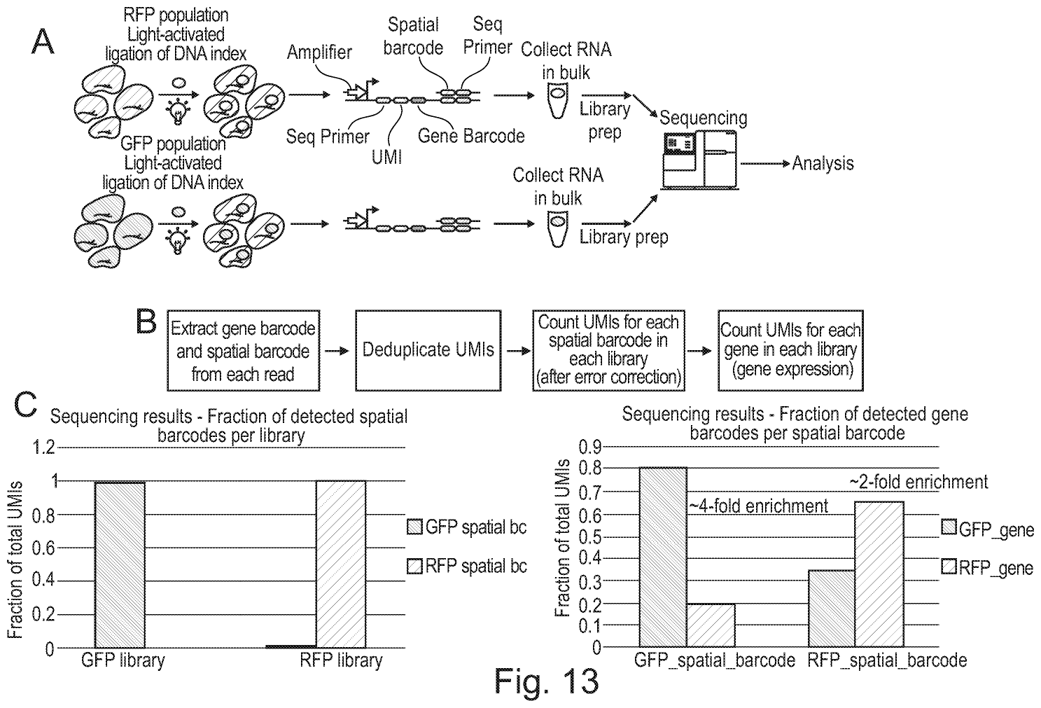

FIG. 13 shows: results of a proof of concept experiment (example 9) aimed at measuring gene expression in cultured cells using the methods of this invention and illumina DNA sequencing as quantification tool. For this experiment we used a pool of detection probes targeting two genes, green fluorescent protein and red fluorescent protein, expressed in two separate cell populations. Each population was barcoded with a different index sequence. The two populations were deconvolved after sequencing by matching the spatial barcodes assigned by the methods of this invention, and the abundance of the detection probes targeting each gene quantified. In a successful experiment, the gene abundance measured by the experiment should correspond to the fluorescent gene expressed in each cell population. (A) scheme of the experiment (B) computational analysis pipeline used to calculate results. (C) left: fraction of reads mapped to each spatial barcode (“GFP population” or “RFP population”) detected into the library produced from GFP or RFP cells. Essentially all reads bear the correct spatial barcode. Right: gene abundance measured for the GFP and RFP detection probes in each cell population. The GFP detection probes are predominantly detected in the GFP population and viceversa, indicating that the protocol can detect gene expression. Detection of RFP proves in the GFP population and viceversa is due to a specific binding of the detection probes

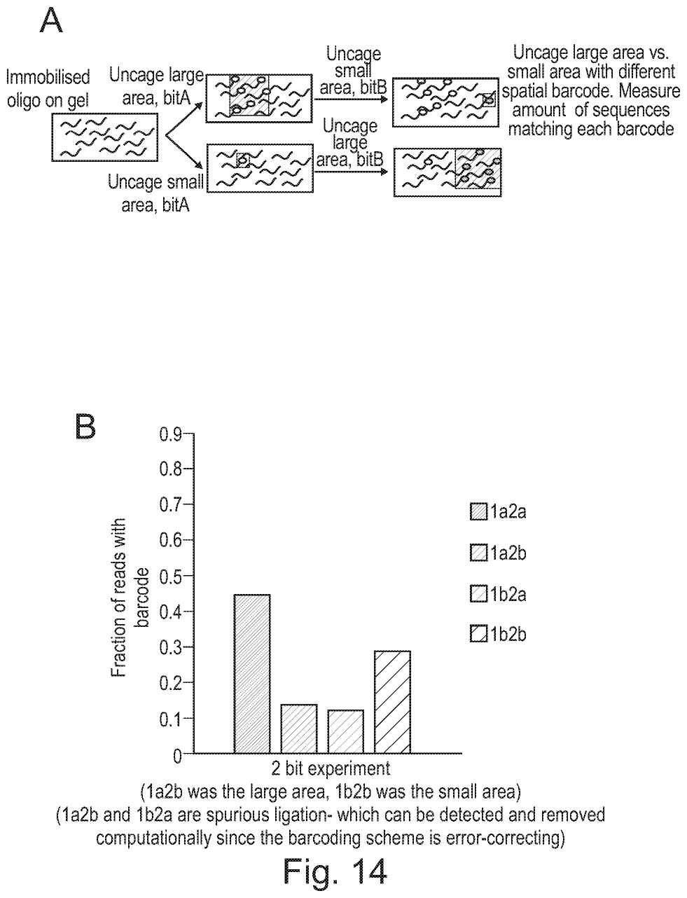

FIG. 14 shows: results on an experiment (example 10) aimed at showing that spatial barcoding can successfully measure the abundance of detection probes bound to different areas of the same tissue (example 9 was done on separate cell populations). A functionalised hydrogel bearing root molecules designed to resemble detection probes is used for this experiment. Areas of different sizes are uncaged and barcoded with a 2-bit spatial barcode using the methods of this invention. The abundance of detection probes in each spatially barcoded area is measured by illumina sequencing by mapping the spatial barcode present in each read. In a successful experiment, the abundance of root molecules measured in each spatially barcoded area should match the area size. (A) scheme of the experiment. (B) results of the quantification. The 2-bit spatial barcodes assigned to the “large” and “small” area were “1a2a” and “1b2b”. The experiment correctly measures more molecules for 1a2a. The other combinations (“1a2b” and “1b2a”) are presumably products of spontaneous uncaging and ligation produced by stray light in the experiment (since the proof of concept experiment could not be done in completely light-proof conditions)

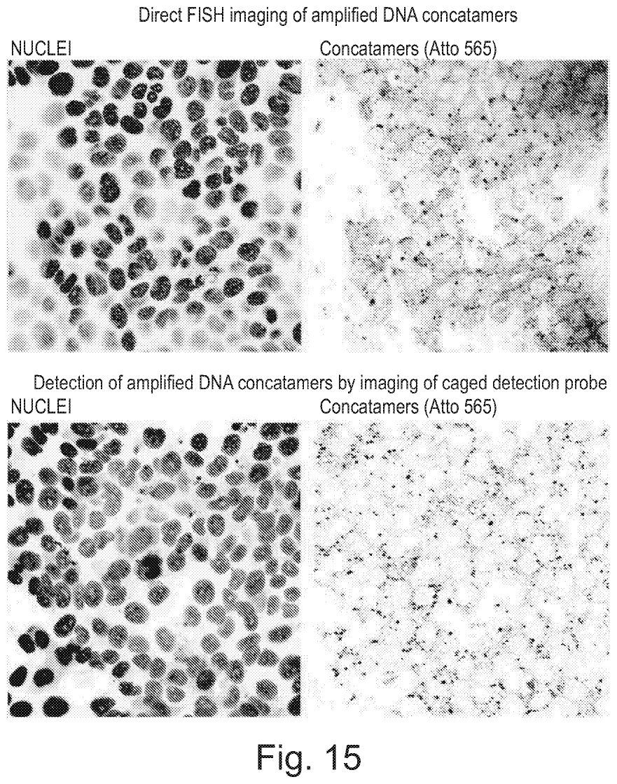

FIG. 15 shows: results of an experiment (example 11) aimed at demonstrating a method of signal amplification. A cell population bearing an expressed barcode in their genome (and therefore expressing a mRNA transcript with the barcode sequence) were subjected to the “STARmap” protocol, producing a DNA concatemer specific for the barcode itself. Detection probes designed according to the methods of this invention were then hybridized to the concatemer and detected by ligation with a photocaged index bearing a fluorescent group. The concatamers were also detected directly by fluorescence in-situ hybridization (FISH) in a separate coverslip. The expected binding pattern is a punctate staining around the cell nucleus. TOP: nuclear stain and concatemer signal from direct FISH binding, BOTTOM: nuclear stain and concatemer signal from the detection probes ligated with a fluorescent index. The binding pattern corresponds, indicating that it's possible to target binding probes (and therefore perform the methods of this invention) on an amplified target produced by methods such as STARmap.

### EXAMPLES

Methods are specified below when used. All oligonucleotides sequences were obtained from Integrated DNA technologies, AtdBio or Biomers, and all the chemicals (unless otherwise specified) from Sigma-Aldrich.

The term ‘cage’ or ‘PC spacer’ throughout refers to a photocleavable spacer modification with the following structure (formula I) as is shown in FIG. 3:

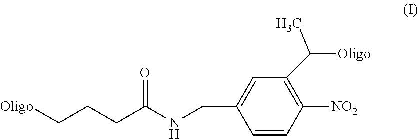

### Example 1

A 75np DNA duplex with a fluorescent 5′ phosphate block capping an 8 nt overhang was produced by mixing the BALI_01 and BALI_02 primers at 10 μM final concentration in 2×SSC buffer, incubating the solution at 95° C. for 2 minutes, and letting it cool down at room temperature (20° C.) for 30 minutes. A second, shorter DNA duplex was produced by the same procedure annealing the BALI_03 and BALI_04 primers.

Immediately after dimerization, the longer duplex was split into several samples and irradiated (or not) with different wavelength of light for increasing durations. Irradiation was produced either by a collimated solid state 405 nm laser with intensity of approximately 100 mw/mm², or by a UV crosslinker (UVP-CL1000) equipped with 365 nm fluorescent bulbs, with the samples at approx. 2 cm from the emitter.

After irradiation, 2 μl of the duplex (corresponding to 20 μmol) were combined with one molar equivalent of the shorter duplex, 10 μl of NEB quick ligase buffer (see below), and 2000 U of T4 ligase (NEB) in a 20 μl reaction for 30 minutes at room temperature (21° C.). After ligation, the samples (plus a control sample including the first duplex alone) were ran on a non-denaturing 12% acrylamide gel in Tris-Borate EDTA buffer. The gel was stained using SYBR-Gold (Thermo Fischer scientific) at 1:10000 dilution in 1×TBS for 30 minutes, and imaged on an Amersham Typhoon imager in the cy2 and cy3 channel. The background/corrected image was produced by dividing the cy3 channel image by the cy2 channel image, in order to remove the bleed-through signal from sybr-gold.

TABLE 1

| | | |
|---|---|---|
| BALI_01 | [Cy3] [cage] CTAGGTCG | SEQ ID |
| | ATGTCGAGAG CAATTAGAGT | NO: 1 |
| | CGCGCCTTAAGATACAGATC | |
| | GGAAGAGCGTCGTGTAG | |
| | CGCGCCTTAAGATAC | |
| BALI_02 | GTATCTTAAGGCGCGACTC | SEQ ID |
| | TAATTGCTCTCGACAT | NO: 2 |
| BALI_03 | GGTAGTAT AGCTACCATG | SEQ ID |
| | | NO: 3 |
| BALI_04 | CGACCTAG CATGGTAGCT | SEQ ID |
| | | NO: 4 |

(“Cage” refers to the photocleavable spacer oligo modification, as shown in FIG. 3, “cy3” refers to a cyanine 3 fluorescent group bound to the 5′ of the molecule)

### Example 2

A solid surface labelled with a detection probe was produced as follows: the BALI_05 oligonucleotide was diluted to 1 μM final concentration in PBS buffer (250 μl per slide). A 1:100 dilution of a 10 mM solution of BS(PEG)9 crosslinker (Pierce) in DMSO was added to the mix, and the resulting solution was spread on a glass slide coated with aminoalkylsilane (Sigma, Silane-Prep) using a coverslip. The slide was incubated for 2 h at 30° C. in a humid chamber, washed for 10 minutes with 0.1% glycine in PBS, and washed several times in PBS.

In order to produce a double-stranded end on the detection probe, the BALI_06 oligonucleotide was diluted to a final 1 μM concentration in 2×SSC and incubated on the slide surface for 5 minutes at 95° C. temperature, followed by 30 minutes at room temperature. The slide was washed three times for 5′ washes in 2×SSC.

The slide functionalised with the double-stranded molecule was imaged on a Leica SP5 confocal microscope equipped with a 30 mW 405 nm solid state laser, an argon laser line at 514, a He—Ne laser at 543 nm, and a solid state 647 nm laser. Cy3 was excited using the 514 and 543 nm laser lines, and the fluorescence signal was captured by a PMT after a 550-600 nm bandpass filter. Cy5 was excited by the 647 nm laser and the relative fluorescence signal captured by a PMT after a 660-750 nm bandpass filter. Once the surface of the slide was identified by detecting the plane of maximum cy3 signal, photorelease was produced by illuminating two region of interest with 100% power of the 405 nm laser for 2 minutes and 5 minutes, respectively. After photorelease, the slide was washed three times for 5′ in 2×SSC.

The BALI_07 and BALI_08 oligos were mixed to a 5 μM final concentration in 2×SSC buffer, heated at 95° C. for 5 minutes, and allowed to cool down at room temperature for 30 minutes. A ligation solution was prepared by mixing: 107.5 μl of ultra-pure water, 125 μl 2× quick ligation mix (NEB), 12.5 ul T4 ligase, high concentration (NEB), and 5 μl (final 100 uM) of BALI_07/08 oligos. The ligation solution was incubated on the slide for 30 minutes at room temperature, followed by three 5′ washes in 2×SSC.

After the first series of washes, the slide was imaged again using the same parameters of the first imaging. Following imaging, the slide was washed further twice for 10 minutes in 0.2×SSC at 50° C., and once in 0.2×SSC at room temperature. The slide was then imaged a third time with the same settings.

TABLE 2

| | | |
|---|---|---|
| BALI_05 | [Cy3] [cage] CTAGGTCG | SEQ ID |
| | ATGTCGAGAG CAATTAGAGT | NO: 5 |
| | CGCGCCTTAAGATAC | |
| | AGATCGGAAGAGCGTCGTGTAG | |
| | CGCGCCTTAAGATAC | |
| | [Aminolink C6] | |
| BALI_06 | GTATCTTAAGGCGCGACTC | SEQ ID |
| | TAATTGCTCTCGACAT | NO: 6 |
| BALI_07 | [Cy5] GGTAGTAT | SEQ ID |
| | AGCTACCATG | NO: 7 |
| BALI_08 | CGACCTAG CATGGTAGCT | SEQ ID |
| | | NO: 8 |

(“Cage” refers to the photocleavable spacer oligo modification as shown in FIG. 3, “cy3” refers to a cyanine 3 fluorescent group bound to the 5′ of the molecule, “cy5” refers to a cyanine 5 fluorescent group bound to the 5′ end of a molecule, ‘aminolink C6’ refers to an NH2 group)

### Example 3

A solid surface labelled with a detection probe was produced as follows: the BALI_09 oligonucleotide was diluted to 1 μM final concentration in PBS buffer (250 ul per slide). A 1:100 dilution of a 10 mM solution of BS(PEG)9 crosslinker (Pierce) in DMSO was added to the mix, and the resulting solution was spread on a glass slide coated with aminoalkylsilane (Sigma, Silane-Prep) using a coverslip. The slide was incubated for 2 h at 30° C. in a humid chamber, washed for 10 minutes with 0.1% glycine in PBS, and washed several times in PBS.

In order to produce a double-stranded end on the detection probe, the BALI_09 oligonucleotide was diluted to a final 1 μM concentration in hybridization buffer (10% ethylene carbonate in 2×SSC) and incubated on the slide surface for 15 minutes at room temperature, followed by two 5′ washes in hybridization solution at room temperature and three washes in 2×SSC at room temperature.

The detection probe bound to the slide was extended by a DNA bridge molecule bearing a photocleavable group and the Alexa-488 fluorophore as follows: the BALI_10 and BALI_11 primers were diluted to a final concentration of 5 μM in 5×SSC buffer, heated at 95 C for 5 minutes, and gradually cooled down to 30° C. on a PCR cycler using a temperature gradient of

−1° C./30″. A ligation solution was prepared by mixing: 107.5 μl of ultra-pure water, 125 ul 2× quick ligation mix (NEB), 12.5 μl T4 ligase, high concentration (NEB), and 5 μl (final 100 μM) of BALI_10/11 oligos. The ligation solution was incubated on the slide for 30 minutes at room temperature, followed by three 5′ washes in 2×SSC

The slide bearing the detection probe extended by the photocleaved DNA bridge molecule was imaged on a Leica SP5 confocal microscope equipped with a 30 mW 405 nm solid state laser, an argon laser line at 488 and 514 nm, a He—Ne laser at 543 nm, and a solid state 647 nm laser. Alexa 488 was excited using the 488 nm laser, and the relative fluorescence signal captured by a PMT after a 510-540 nm bandpass filter. Atto 568 was excited by the 543 nm laser line and the relative fluorescence signal captured by a PMT after a 560-600 nm bandpass filter. Cy5 was excited by the 647 nm laser and the relative fluorescence signal captured by a PMT tube after a 660-750 nm bandpass filter. Once the surface of the slide was identified by detecting the plane of maximum Alexa 488 signal, photorelease was produced by illuminating two rectangular region of interest with 100% power of the 405 nm laser for 5 minutes each. After photorelease, the slide was washed three times for 5′ in 2×SSC.

For the first spatial barcoding step, a double-stranded index composed of the BALI_12 and BALI_13 primers was produced by annealing the two oligonucleotides at a final concentration of 5 μM as described before. A second ligation reaction was prepared as described before and incubated on the slide for 30′ at room temperature. After the ligation, the slide was washed for three times in 2×SSC at room temperature. The slide was imaged as above. Light was used to photorelease the photocleavable group only on one of the two barcoded areas for the same time and using the same power described above.

For the second spatial barcoding step, a double-stranded index composed of the BALI_13 and BALI_14 primers was produced by annealing the two oligonucleotides at a final concentration of 5 μM as described before. A third ligation reaction was prepared as described before and incubated on the slide for 30′ at room temperature. After the ligation, the slide was washed for three times in 2×SSC at room temperature and for three times for 5′ in 0.2×SSC at 50° C. The slide was imaged as above for a third time with the same settings

TABLE 3

| | | |
|---|---|---|
| BALI_09 | GGTAGTAT | SEQ ID |
| | ATGTCGAGAGCTAGC | NO: 9 |
| | CGCGCCTTAAGATAC CAATTAGAGT | |
| | AGATCGGAAGAGCGTCGTGTAG | |
| | CTCCCTATAGTGAGTCGTATTA | |
| | CTAGCTAGCG [Aminolink C6] | |
| BALI_10 | GCTAGCTCTCGACAT | SEQ ID |
| | | NO: 10 |
| BALI_11 | [Atto488] [cage] | SEQ ID |
| | GTACCTGT | NO: 11 |
| | CAGCTACCATG | |
| BALI_12 | [5′ PHOS] ATACTACC | SEQ ID |
| | CATGGTAGCTG | NO: 12 |
| BALI_13 | [Cy5] [cage] GTATCGAG | SEQ ID |
| | CTCTATACAC | NO: 13 |
| BALI_14 | [5′ PHOS] ACAGGTAC | SEQ ID |
| | GTGTATAGAG | NO: 14 |
| BALI_15 | [Atto565] [cage] | SEQ ID |
| | GTGAGCGT | NO: 15 |
| | CGGACACCTAC | |
| BALI_16 | [5′ PHOS] CTCGATAC | SEQ ID |
| | GTAGGTGTCCG | NO: 16 |

(“Cage” refers to the photocleavable spacer oligo modification as shown in FIG. 3, “Atto488” refers to a the atto488 green fluorescent group bound to the 5′ of the molecule, “Atto565” refers to the atto565 red fluorescent group bound to the 5′ end of a molecule, “cy5” refers to a cyanine 5 fluorescent group bound to the 5′ end of a molecule, ‘aminolink C6’ refers to an NH2 group, 5′PHOS refers to phosphate)

### Example 4

A cell monolayer bound to a detection probe was produced as follows: U2OS cells (ATCC® HTB-96) were grown until confluence on a circular #1.5 coverslip of 40 mm diameter, previously coated with 10 mg/ml poly-L-lysine in PBS for 12 h. Cells were grown in Dulbecco's modified Eagle's medium (DMEM) supplemented with 10% Fetal Bovine Serum and 1% Penicillin/Streptomycin antibiotics. Prior to the experiment, cells were fixed in 4% paraformaldehyde in PBS for 15 minutes at room temperature and washed 3 times for 5 minutes at room temperature.

To crosslink a detection probe to the cell surface, the BALI_05 oligonucleotide was diluted to 1 μM final concentration in PBS buffer (250 μl per slide). A 1:100 dilution of a 10 mM solution of BS(PEG)9 crosslinker (Pierce) in DMSO was added to the mix, and the resulting solution was spread on the coverslip containing the cells. The slide was incubated for 12 h at room temperature (21° C.), washed for 10 minutes with 0.1% glycine in PBS, and washed twice for 5 minutes in 2×SSC.

In order to produce a double-stranded end on the detection probe, the BALI_06 oligonucleotide was diluted to a final 1 μM concentration in hybridization buffer (10% ethylene carbonate in 2×SSC) and incubated on the slide surface for 15 minutes at room temperature, followed by two 5′ washes in hybridization solution at room temperature and three washes in 2×SSC at room temperature.

The slide functionalised with the double-stranded molecule was imaged on a Leica SP5 confocal microscope equipped with a 30 mW 405 nm solid state laser, an argon laser line at 514, a He—Ne laser at 543 nm, and a solid state 647 nm laser. Cy3 was excited using the 514 and 543 nm laser lines, and the fluorescence signal was captured by a PMT after a 550-600 nm bandpass filter. Cy5 was excited by the 647 nm laser and the relative fluorescence signal captured by a PMT after a 660-750 nm bandpass filter. Once the surface of the slide was identified by detecting the plane of maximum cy3 signal, photorelease was produced by illuminating a region of interest with 100% power of the 405 nm laser for 5 minutes. After photorelease, the slide was washed three times for 5′ in 2×SSC.

The BALI_07 and BALI_08 oligos were mixed to a 5 μM final concentration in 2×SSC buffer, heated at 95° C. for 5 minutes, and allowed to cool down at room temperature for 30 minutes. A ligation solution was prepared by mixing: 107.5 μl of ultra-pure water, 125 ul 2× quick ligation mix (NEB), 12.5 ul T4 ligase, high concentration (NEB), and 5 μl (final 100 uM) of BALI_07/08 oligos. The ligation solution was incubated on the slide for 30 minutes at room temperature, followed by three 5′ washes in 2×SSC.

After the first series of washes, the slide was imaged again using the same parameters of the first imaging, only in the cy5 channel.

### Example 5

Two index molecules were produced by annealing each of the following oligonucleotides:

TABLE 4

| | | | |
|---|---|---|---|
| BALI_017 | [phosphate] | Overhang A | SEQ ID |
| | GTGCGTGCACCACAGTCG | 6 nt | NO: 17 |
| BALI_018 | [phosphate] | Overhang C | SEQ ID |
| | GTGACCGCACCACAGTCG | 6 nt | NO: 18 |
| BALI_019 | ACGCACCGGGACTCGTGC | Overhang B | SEQ ID |
| | | 6 nt | NO: 19 |
| BALI_020 | GGTCACCGGGACTCGTGC | Overhang D | SEQ ID |
| | | 6 nt | NO: 20 |
| BALI_021 | [phosphate] | Overhang A | SEQ ID |
| | GTGACGTGCACCACAGTCG | 7 nt | NO: 21 |
| BALI_022 | [phosphate] | Overhang C | SEQ ID |
| | GTGGACCGCACCACAGTCG | 7 nt | NO: 22 |
| BALI_023 | ACGTCACCGGGACTCGTGC | Overhang B | SEQ ID |
| | | 7 nt | NO: 23 |
| BALI_024 | GGTCCACCGGGACTCGTGC | Overhang D | SEQ ID |
| | | 7 nt | NO: 24 |

With the BALI_025 oligonucleotide:

| | | |
|---|---|---|
| BALI_025 | GCACGAGTCCCG | SEQ ID NO: 25 |

In each case, the forward and reverse oligonucleotides were diluted to a final concentration of 5 μM in TE buffer, incubated for 5 minutes at 95° C., and cooled down to 25° C. in a PCR cycler using a temperature gradient of −1 C/30 seconds.

A ligation mix was prepared by mixing the following: 7 μl ultrapure water, 10 μl 2× quick ligation mix (NEB), 1 μl T4 ligase, high concentration (NEB), and 1 μl each of the two index molecules to be tested (final concentration 200 nM)

The reaction was incubated for 30 minutes at room temperature. The samples were then diluted in loading buffer and ran on a non-denaturing acrylamide gel. The gel was stained using SYBR-Gold (Thermo Fischer scientific) at 1:10000 dilution in 1×TBS for 30 minutes, and imaged on an Amersham Typhoon imager in the cy2 channel.

### Example 6

Magnetic beads were functionalised with a detection probe as follows. The BALI_26 oligonucleotide was desalted using a GE life sciences Illustra microspin G-25 column according to the supplier instructions. 50 μl of a 100 μM oligo were used for the desalting. 200 μl of Dynabeads M270 carboxylic acid (Thermo Scientific) were washed twice in 25 mM MES buffer at pH 4.7 and resuspended in 50 μl of 100 mM MES buffer at pH 4.7. The bead slurry was supplemented with 30 μl of the desalted BALI_26 oligo and 20 μl of ultrapure water. This mix (100 μl) was added to 100 ul 25 mM MES buffer at pH 4.7 in which 1 mg EDC (1-ethyl-3-(3-dimethylaminopropyl)carbodiimide hydrochloride) had been previously resuspended. The reaction was incubated for 12 h at 4° C. on a tube rotator, and the beads were washed 4 times for 5′ each in 50 mM tris pH 7.4+0.1% Tween 20 to quench the reaction.

The BALI_26 oligonucleotide encodes a detection probe ending with a “A” overhang, 6 nt.

In order to produce a double-stranded molecule at the end of the detection probe, 140 μl of the functionalised beads were resuspended in 2×SSC and supplemented with 14 μl of 100 uM BALI_10 oligo (see examples above). The resulting mixture was incubated at 95° C. for 5 minutes and allowed to cool down to room temperature for 30 minutes on a rotator.

Different index molecules were produced by annealing the oligonucleotides specified below. In each case, the oligos were annealed by mixing them at a final concentration of 5 uM in TE buffer, heating them to 95° C. for 5 minutes, and cooling them down to 25° C. in a PCR cycler with a thermal gradient of −1° C./30 seconds.

TABLE 5

| Sample | BALI_19 | Index molecule exposing the following barcodes: |
|---|---|---|
| 1 | and | Side facing the existing spatial barcode: B 6 nt |
| | BALI_25 | (match for the BALI_26 oligo) |
| | | Side exposed to the addition of new indices: no |
| | | overhang |
| Sample | BALI_24 | Index molecule exposing the following barcodes: |
| 2 | and | Side facing the existing spatial barcode: D 7 nt |
| | BALI_25 | (no match) |
| | | Side exposed to the addition of new indices: no |
| | | overhang |
| Sample | BALI_27 | Index molecule exposing the following barcodes: |
| 3 | and | Side facing the existing spatial barcode: B 6 nt |
| | BALI_28 | (match for the BALI_26 oligo) |
| | | Side exposed to the addition of new indices: C 7 nt |
| Sample | BALI_29 | Index molecule exposing the following barcodes: |
| 4 | and | Side facing the existing spatial barcode: D 7 nt (no |
| | BALI_30 | match) Side exposed to the addition of new indices: |
| | | A 6 nt |
| Sample | As sample | |
| 5 | 3 | |
| Sample | As sample | |
| 6 | 3 | |

The functionalised beads with the annealed BALI_10 oligo were captured on a magnetic tube rack and resuspended in a ligation solution comprising: 8 μl ultrapure water, 10 μl 2× quick ligation mix (NEB), 1 μl T4 ligase, high concentration (NEB), and 1 μl of 5 μM annealed oligo as per scheme above (final 100 nM). A seventh reaction was assembled as negative control without any index molecule. Each reaction was incubated for 30 minutes at room temperature with rotation.

Following the first ligation reaction, the beads were washed 3 times for 5′ in 2×SSC.

A second ligation mix was then assembled for samples 5 and 6 according to the scheme below

TABLE 7

| | | |
|---|---|---|
| Sample | BALI_29 | Index molecule exposing the following barcodes: |
| 5 | and | Side facing the existing spatial barcode: D 7 nt |
| | BALI_30 | (match for the index added during the first ligation) |
| | | Side exposed to the addition of new indices: A 6 nt |
| Sample | BALI_27 | Index molecule exposing the following barcodes: |
| 6 | and | Side facing the existing spatial barcode: B 6 nt (no |
| | BALI_28 | match) Side exposed to the addition of new indices: |
| | | C 7 nt |

The ligation reaction was assembled as indicated above and incubated for the same time with rotation. After ligation, the beads were washed for 3 times for 5′ each in 2×SSC.

Following the second ligation on samples 5 and 6, all seven samples were subjected to signal amplification using T7 RNA in-vitro transcription. For each sample, the beads were captured using a magnet tube rack and resuspended in 100 μl of hybridization buffer (10% ethylene carbonate in 2×SSC) supplemented with 1 μM final of T7 promoter oligo. The beads were incubated in this solution for 30 minutes at room temperature with rotation and washed 3 times for 5 minutes in hybridization solution.

Following the last wash, beads from each sample were resuspended in 50 μl of T7 transcription solution comprising: 10 μl of 5× transcription buffer (Promega), 2 μl of RNAseOUT nuclease inhibitor (Thermo Fisher), 2 μl of T7 polymerase (Promega), 5 μl 100 mM DTT, 10 μl of 2.5 mM NTP mix, and 21 μl ultrapure water. The reaction was incubated for 3 h at 37° C. with shaking.

Following the reaction, the beads from each sample were immobilized using a magnetic tube rack, and the supernatant containing the amplified detection probes connected to the spatial barcode was collected, mixed with 2× denaturing RNA loading buffer, and ran on a 15% TBE-Urea poly-acrylamide gel.

TABLE 8

| | | | SEQ ID |
|---|---|---|---|
| Name | Sequence | Notes | NO |
| BALI_26 | [phosphate] GTGCGT | | SEQ ID |
| | ATGTCGAGAGCTAGC | | NO: 26 |
| | CGCGCCTTAAGATAC | | |
| | CAATTAGAGT | | |
| | AGATCGGAAGAGCGT | | |
| | CGTGTAGCTCCCTAT | | |
| | AGTGAGTCGTATTA | | |
| | CTAGCTAGCG | | |
| | [aminolink C6] | | |
| BALI_27 | ACGCACCGGGACTCG | Overhang | SEQ ID |
| | TGC | B 6 nt | NO: 27 |
| BALI_28 | [phosphate] GTGG | Overhang | SEQ ID |
| | ACCGCACGAGTCCCG | C 7 nt | NO: 28 |
| BALI_29 | GGTCCACCGACTG | Overhang | SEQ ID |
| | TGGTGC | D 7 nt | NO: 29 |
| BALI_30 | [phosphate] GTGC | Overhang | SEQ ID |
| | GTGCACCACAGTCG | A 6 nt | NO: 30 |
| T7 | TAATACGACTCAC | | SEQ ID |
| promoter | TATAGGGAG | | NO: 31 |

(‘aminolink C6’ refers to an NH2 group)

### Example 7

Cyclic Barcoding on Solid Gel Beads.

TABLE 9

| | SEQ ID | Name in | |
|---|---|---|---|
| Name | NO. | figure | Sequence |
| BALI_31 | 32 | BL_91 | [NH2] CTACTACGGCTAGCC |
| | | | TAATACGACTCACTATAGGGAG |
| | | | ACTCTAATTG |
| | | | GTATAGAATTCCGCG |
| | | | GCTAGCTCTCGACAT |
| BALI_32 | 33 | BL_92 | [phosphate] GTGCGT |
| | | | ATGTCGAGAGCTAGC |
| | | | CGCGGAATTCTATAC |
| | | | CAATTAGAGT |
| | | | CTCCCTATAGTGAGT |
| | | | CGTATTA GGC [cy5] |
| BALI_33 | 34 | BL045 | ACGCACCGGGACTCGTGC |
| BALI_34 | 35 | BL046 | [phosphate] |
| | | | GTGGACCGCACGAGTCCCG |
| BALI_35 | 36 | BL047 | [phosphate] |
| | | | GTGCGTGCACGTATGGCG |
| BALI_36 | 37 | BL048 | GGTCCACCGCCATACGTGC |

(“cy5” refers to a cyanine 5 fluorescent group)

This protocol mimics the process of producing a spatial barcode on detection probes. A double stranded DNA root molecule bearing a fluorophore is attached to an agarose gel bead, which has mechanical features compatible with those of the gel produced during the in-situ labelling protocol. Multiple cycles of ligation are then performed using different index sequences. The efficiency of each ligation step is measured by densitometry on denaturing acrylamide electrophoresis.

Oligo-modified agarose beads were prepared by reacting NHS-modified sepharose beads (GE Healthcare) with the BALI_31 oligo ad a final concentration of 25 uM in 50 mM Sodium Borate buffer, pH 8.5, for 4 h at room temperature. The reaction was stopped by adding ⅕th volume of 1M Tris-HCl pH 8, followed by several washes in Tris-Edta buffer (100 mM Tris-HCl pH 8, 2.5 mM EDTA). For every wash, beads were pelleted by centrifuging them at

Oligos BALI_32, BALI_34 and BALI_35 were phosphorylated by incubating them at 37 C for 30 minutes, at a concentration of 10 uM, in a reaction buffer composed of 200 uM ATP, 1×PNK reaction buffer (NEB), and 10 U T4 polynucleotide kinase (NEB), and purified through a G25 sepharose spin column (Illustra microspin). Following this, oligos BALI_33 and BALI_34 and oligos BALI_35 and BALI_36 were annealed by mixing them in Tris-EDTA buffer (TE) at a final concentration of 5 uM, heating up at 95 C for 2 minutes, and cooling down to RT for 30 minutes.

The oligo-conjugated agarose beads (20 ul of 25% bead slurry for each sample) were hybridized with the root oligo BALI_32 by incubating them in hybridization buffer (10% Ethylene Carbonate, 2×SSC), supplemented with the root oligo at 1 uM final concentration, at room temperature for 30 minutes. After this, the beads were washed three times for 10 minutes in hybridization buffer, and three times for 5 minutes in 2×SSC.

The first cycle of ligation was performed by incubating the bead sample in 20 ul a reaction buffer composed by 1× T4 ligase buffer (NEB), 0.75 uM annealed oligos BALI_33 and BALI_34, and 100/ul U T4 DNA ligase (NEB) for 30 minutes at room temperature. Following the ligation, samples were washed twice in 2×SSC for 5 minutes each. After this, more cycles of ligation (up to seven in total) were performed as above, alternating annealed oligos BALI_35/36 and BALI_33/34.

The final ligated product was purified by washing the bead samples twice in 2×SSC for 5 minutes, resuspending them in 20 ul 2×SSC, and adding 20 ul of 2× denaturing RNA loading buffer (95% Formamide, 5% TBE, 10 mg/ml bromophenol blue). The samples were heated at 95 C for 5 minutes, spun quickly to pellet beads, and the supernatant was collected and loaded on a 8% denaturing polyacrylamide gel for analysis. Beads subjected to one, two, three, four, five, six or seven ligation cycles were compared, and quantified by densitometry after imaging of the gel, measuring the ligation efficiency. Results are shown in FIG. 11.

### Example 8

Comparison of Ligation Efficiency for Different Index Sequences.

TABLE 10

| | SEQ ID | Name in | | |
|---|---|---|---|---|
| Name | NO: | figure | Sequence | notes |
| BALI_37 | 38 | BL174 | [phosphate] GTGCGTGCACCACAGTCG | Barcode 1 |
| BALI_38 | 39 | BL175 | GGTCCACCGACTGTGGTGC | Barcode 1 |
| BALI_39 | 40 | BL176 | [phosphate] GTGCGTGCACCGACCTCG | Barcode 2 |
| BALI_40 | 41 | BL177 | GGTCCACCGAGGTCGGTGC | Barcode 2 |
| BALI_41 | 42 | BL178 | [phosphate] GTGCGTGCACCGTGTACG | Barcode 3 |
| BALI_42 | 43 | BL179 | GGTCCACCGTACACGGTGC | Barcode 3 |
| BALI_43 | 44 | BL180 | [phosphate] GTGCGTGCACCTAGATCG | Barcode 4 |
| BALI_44 | 45 | BL181 | GGTCCACCGATCTAGGTGC | Barcode 4 |
| BALI_45 | 46 | BL182 | [phosphate] GTGCGTGCACGAGTCCCG | Barcode 5 |
| BALI_46 | 47 | BL183 | GGTCCACCGGGACTCGTGC | Barcode 5 |
| BALI_47 | 48 | BL184 | [phosphate] GTGCGTGCACGGACGACG | Barcode 6 |
| BALI_48 | 49 | BL185 | GGTCCACCGTCGTCCGTGC | Barcode 6 |
| BALI_49 | 50 | BL186 | [phosphate] GTGCGTGCACGTATGGCG | Barcode 7 |
| BALI_50 | 51 | BL187 | GGTCCACCGCCATACGTGC | Barcode 7 |
| BALI_51 | 52 | BL188 | [phosphate] GTGCGTGCACTATCTGCG | Barcode 8 |
| BALI_52 | 53 | BL189 | GGTCCACCGCAGATAGTGC | Barcode 8 |
| BALI53 | 54 | BL190 | [phosphate] GTGCGTGCACTCAGGTCG | Barcode 9 |
| BALI_54 | 55 | BL191 | GGTCCACCGACCTGAGTGC | Barcode 9 |
| BALI_55 | 56 | BL192 | [phosphate] GTGCGTGCAGAGATGACG | Barcode 10 |
| BALI_56 | 57 | BL193 | GGTCCACCGTCATCTCTGC | Barcode 10 |
| BALI_57 | 58 | BL230 | [phosphate] GTGCGTGCCACACATCCG | Barcode 11 |
| BALI_58 | 59 | BL231 | GGTCCACCGGATGTGTGGC | Barcode 11 |
| BALI_59 | 60 | BL232 | [phosphate] GTGCGTGCCACATGCTCG | Barcode 12 |
| BALI_60 | 61 | BL233 | GGTCCACCGAGCATGTGGC | Barcode 12 |
| BALI_61 | 62 | BL234 | [phosphate] GTGCGTGCCACGGTAGCG | Barcode 13 |
| BALI_62 | 63 | BL235 | GGTCCACCGCTACCGTGGC | Barcode 13 |
| BALI63 | 64 | BL236 | [phosphate] GTGCGTGCCAGCCGATCG | Barcode 14 |
| BALI_64 | 65 | BL237 | GGTCCACCGATCGGCTGGC | Barcode 14 |
| BALI_65 | 66 | BL238 | [phosphate] GTGCGTGCCAGTATAGCG | Barcode 15 |
| BALI_66 | 67 | BL239 | GGTCCACCGCTATACTGGC | Barcode 15 |
| BALI_67 | 68 | BL240 | [phosphate] GTGCGTGCTACACCGGCG | Barcode 16 |
| BALI_68 | 69 | BL241 | GGTCCACCGCCGGTGTAGC | Barcode 16 |
| BALI_69 | 70 | BL242 | [phosphate] GTGCGTGCTACTCGTCCG | Barcode 17 |
| BALI_70 | 71 | BL243 | GGTCCACCGGACGAGTAGC | Barcode 17 |
| BALI_71 | 72 | BL244 | [phosphate] GTGCGTGCTAGACTCCCG | Barcode 18 |
| BALI_72 | 73 | BL245 | GGTCCACCGGGAGTCTAGC | Barcode 18 |
| BALI73 | 74 | BL246 | [phosphate] GTGCGTGCTAGCTGAGCG | Barcode 19 |
| BALI_74 | 75 | BL247 | GGTCCACCGCTCAGCTAGC | Barcode 19 |
| BALI_75 | 76 | BL248 | [phosphate] GTGCGTGCTCACTCACCG | Barcode 20 |
| BALI_76 | 77 | BL249 | GGTCCACCGGTGAGTGAGC | Barcode 20 |

This experiment was performed to compare the relative ligation efficiency for 20 pair of different spatial indexes. The experiment was performed by ligating each pair of oligonucleotides forming a barcode in position “2” of a growing spatial barcode. The overhang sequences used for ligation are identical for all barcodes, and corresponding to those used for oligos BALI_35 and BALI_36.

Ligation was performed on beads using the same protocol described for “cyclic barcoding on solid gel beads” above. Agarose beads conjugated to BALI_31 and hybridized with BALI_32 were first ligated with annealed oligos BALI_33/34, and then (for the second cycle) with a the pair of annealed oligos corresponding to each barcode (i.e. BALI_37/38 for barcode 1).

Following the second ligation, the samples were analysed by denaturing polyacrylamide gel electrophoresis and quantified by densitometry as described above, and the ligation efficiency of the second ligation cycle measured for each barcode. Results are shown in FIG. 12.

### Example 9

Light-Dependent Barcoding Gene Expression Measurements Through BALI on Cells.

In this experiment, cultured cells expressing either green fluorescent protein (GFP) or red fluorescent protein (RFP) were plated on two separate coverslips, and subjected to our protocol for light-dependent barcoding and gene expression measurement. This was done with a library of detection probes including sequences targeting both the GFP and RFP genes (BALI_77 to BALI_84), and using light to barcode such probes with one of two different spatial barcodes. Spatial barcode 1 was used to label GFP cells, whereas spatial barcode 2 was used to label RFP cells. Illumina sequencing was then used to measure how many detection probes targeting GFP/RFP were present in each spatially barcoded population.

4t1 mouse tumour cells expressing GFP or RFP were cultured on #1.5 thickness glass coverslips functionalised first with BIND-silane (GE Healthcare), and then overnight with 0.01% poly-L-lysine in complete culture medium (DMEM, 10% fetal bovine serum). Prior to the experiment, cells were fixed in 4% paraformaldehyde for 15 minutes, washed in PBS, and permeabilised in 0.5% Triton X-100 in phosphate-buffered saline (PBS) for 10 minutes.

The detection probes were diluted in encoding hybridization buffer (2×SSC buffer, 30% formamide, 10% dextran sulphate, 1 mg/ml yeast tRNA, 1:100 NEB murine ribonuclease inhibitor) at a final concentration of 1 uM, and the sample was diluted in the resulting mix for 48 h at 37 C in a humidified chamber. After the hybridization, the sample was washed twice at 47 C for 30 minutes in encoding wash buffer (2×SSC, 30% formamide), and twice at room temperature for 5 minutes in 2×SSC.

A thin hydrogel was cast over the cells by coating the coverslips with a 80 ul drop of degassed hydrogel buffer (4% 19:1 acrylamide:bis-acrylamide mix, 0.3M NaCl, 60 mM Tris-HCl pH 8, 0.05% TEMED, 0.05% Ammonium persulfate) and incubating for 1 h at room temperature. The samples were then digested in digestion buffer (2% SDS, 50 mM tris-HCl pH 8, 0.5% Triton X-100, 1:100 NEB Proteinase K enzyme) overnight at 37 C in a humidified chamber. After the clearing, the coverslips were washed three times for 1 h in 2×SSC, then washed in secondary hybridization buffer (10% Ethylene Carbonate, 2×SSC) for 5 minutes, and hybridized with the BALI_85 oligo (10 nM final concentration, diluted in secondary hybridization buffer) for 15 minutes at room temperature. Finally, samples were washed once in secondary hybridization buffer and once in SSC for 5 minutes each.

Uncaging of the detection probes was performed on a leica SP5 confocal microscope equipped with a 30 mW 405 nm laser, using a 10× objective and 100% laser power. Uncaging was done for 5 minutes on 5 field of views (approx. 1 mm2 each) per sample. Following uncaging, samples were ligated with either spatial barcode 1 or spatial barcode 2 by first annealing the BALI_86 and BALI_87 barcodes or BALI_88 and BALI_89 barcodes (by diluting them in 5×SSC at 5 uM concentration, heating at 95 C for 5 minutes and cooling down slowly to room temperature over 30 minutes), and then incubating them for 30 minutes at room temperature in a ligation mix composed by 1×NEB quick ligation buffer, 100 U/ul T4 DNA ligase, and 100 nM annealed spatial barcode.

Following the ligation step, the hydrogel including the cells was scraped from the coverslips, transferred to a 1.5 ml tube, and diluted in 500 ul 0.4M NaCl. DNA was released by vortexing for 1 h at high speed and purified by ethanol precipitation.

The precipitated DNA (including the barcoded detection probes) was used to produce an illumina sequencing library by two successive rounds of PCR, first using the BALI_90 and BALI_91 primer and the Q5 enzyme from NEB (standard protocol) and the using the Illumina universal forward truseq primer and indexed DNA LT reverse truseq primers (indexes 006 and 012) and the NEB phusion enzyme (standard protocol).

The libraries were sequenced using an Illumina MiSeq sequencer (paired end 150 reads) and analysed through a bioinformatic pipeline developed in the python programming language, which is briefly schematised in additional FIG. 13B.

TABLE 11

| | | SEQ ID |
|---|---|---|
| Name | Sequence | NO: |
| BALI_80 | [CAGE] GTGACC ATGTCGAGAGCTAGC NNNNNNNNNNNNNNN CATCGTGAGT | SEQ |
| | AGATCGGAAGAGCGTCGTGTAG CTCCCTATAGTGAGTCGTATTA CTAGCTAGCG | ID |
| | TTGAAGAAGATGGTGCGCTCCTGGACGTAGCCTTCG | NO: 78 |
| BALI_80 | [CAGE] GTGACC ATGTCGAGAGCTAGC NNNNNNNNNNNNNNN CATCGTGAGT | SEQ |
| | AGATCGGAAGAGCGTCGTGTAG CTCCCTATAGTGAGTCGTATTA CTAGCTAGCG | ID |
| | CTTGAAGTCGATGCCCTTCAGCTCGATGCGGTTCAC | NO: 79 |
| BALI_80 | [CAGE] GTGACC ATGTCGAGAGCTAGC NNNNNNNNNNNNNNN CATCGTGAGT | SEQ |
| | AGATCGGAAGAGCGTCGTGTAG CTCCCTATAGTGAGTCGTATTA CTAGCTAGCG | ID |
| | TGCTTGTCGGCCATGATATAGACGTTGTGGCTGTTG | NO: 80 |
| BALI_80 | [CAGE] GTGACC ATGTCGAGAGCTAGC NNNNNNNNNNNNNNN CATCGTGAGT | SEQ |
| | AGATCGGAAGAGCGTCGTGTAG CTCCCTATAGTGAGTCGTATTA CTAGCTAGCG | ID |
| | CATGTGATCGCGCTTCTCGTTGGGGTCTTTGCTCAG | NO: 81 |
| BALI_81 | [CAGE] GTGACC ATGTCGAGAGCTAGC NNNNNNNNNNNNNNN GGTACAGATG | SEQ |
| | AGATCGGAAGAGCGTCGTGTAG CTCCCTATAGTGAGTCGTATTA CTAGCTAGCG | ID |
| | GATGGCCATGTTATCCTCCTCGCCCTTGCTCACCAT | NO: 82 |
| BALI_82 | [CAGE] GTGACC ATGTCGAGAGCTAGC NNNNNNNNNNNNNNN GGTACAGATG | SEQ |
| | AGATCGGAAGAGCGTCGTGTAG CTCCCTATAGTGAGTCGTATTA CTAGCTAGCG | ID |
| | CTCGGGGAAGGACAGCTTCAAGTAGTCGGGGATGTC | NO: 83 |
| BALI_80 | [CAGE] GTGACC ATGTCGAGAGCTAGC NNNNNNNNNNNNNNN GGTACAGATG | SEQ |
| | AGATCGGAAGAGCGTCGTGTAG CTCCCTATAGTGAGTCGTATTA CTAGCTAGCG | ID |
| | CTTGTAGGTGGTCTTGACCTCAGCGTCGTAGTGGCC | NO: 84 |
| BALI_84 | [CAGE] GTGACC ATGTCGAGAGCTAGC NNNNNNNNNNNNNNN GGTACAGATG | SEQ |
| | AGATCGGAAGAGCGTCGTGTAG CTCCCTATAGTGAGTCGTATTA CTAGCTAGCG | ID |
| | CTGTTCCACGATGGTGTAGTCCTCGTTGTGGGAGGT | NO: 85 |
| BALI_85 | GCTAGCTCTCGACAT | SEQ |
| | | ID |
| | | NO: 86 |
| BALI_86 | AGACGTGTGCTCTTCCGATCTCAGCTACCATG | SEQ |
| | | ID |
| | | NO: 87 |
| BALI_87 | GGTCACCATGGTAGCTGAGATCGGAAGAGCACACGTCT | SEQ |
| | | ID |
| | | NO: 88 |
| BALI_88 | AGACGTGTGCTCTTCCGATCTGACAATGAGGC | SEQ |
| | | ID |
| | | NO: 89 |
| BALI_89 | GGTCACGCCTCATTGTCAGATCGGAAGAGCACACGTCT | SEQ |
| | | ID |
| | | NO: 90 |
| BALI_90 | AGACGTGTGCTCTTCCGATCT | SEQ |
| | | ID |
| | | NO: 91 |
| BALI_91 | TAATACGACTCACTATAGGGAGCTACAC | SEQ |
| | | ID |
| | | NO: 92 |
| Illumina | AATGATACGGCGACCACCGAGATCTACACTCTTTCCCTACACGA | SEQ |
| universal | CGCTCTTCCGATCT | ID |
| FW | | NO: 93 |
| Truseq | | |
| Illumina | CAAGCAGAAGACGGCATACGAGATATTGGCGTGACTGGAGTTCA | SEQ |
| Truseq | GACGTGTGCTCTTCCGATC | ID |
| LT rev. | | NO: 94 |
| index | | |
| 006 | | |
| Illumina | CAAGCAGAAGACGGCATACGAGATTACAAGGTGACTGGAGTTCA | SEQ |
| Truseq | GACGTGTGCTCTTCCGATC | ID |
| LT rev. | | NO: 95 |
| index | | |
| 012 | | |

(“cage” refers to the Photocleavable spacer modification as shown in FIG. 3, ‘N’ refers to any nucleotide of A, T, G, or C)

### Example 10

Spatial Indexing and Assessment of Quantification Capability on Functionalised Hydrogel.

TABLE 12

| | SEQ ID | | |
|---|---|---|---|
| Name | NO: | Sequence | Notes |
| BALI_ | 96 | [acrylate] cgacatcggca | |
| 92 | | agCTACTACGGCTAGCC | |
| | | TAATACGACTCACTATAGGGAG | |
| | | ACTCTAATTG GTATAGAATTCCGCG | |
| | | GCTAGCTCTCGACAT | |
| BALI_ | 97 | [phosphate] GTGACGT | |
| 93 | | ATGTCGAGAGCTAGC CGCGGAATTCTATAC | |
| | | CAATTAGAGT | |
| | | CTCCCTATAGTGAGTCGTATTA GGC | |
| BALI_ | 98 | [cy5][cage] GaGCGTgcaccacagtcg | Bit0 |
| 94 | | | |
| BALI_ | 99 | ACGTCACcgactgtggtgc | Bit0 |
| 95 | | | |
| BALI_ | 100 | [cy3][cage] GTGGACCgcacgtatggcg | 1a |
| 96 | | | |
| BALI_ | 101 | ACGCtCcgccatacgtgc | 1a |
| 97 | | | |
| BALI_ | 102 | [cy3][cage] GTGGACCgcacgagtcccg | 1b |
| 98 | | | |
| BALI_ | 103 | ACGCtCcgggactcgtgc | 1b |
| 99 | | | |
| BALI_ | 104 | AGACGTGTGCTCTTCCGAT | 2a |
| 100 | | CTACTGAGGTGAGC | |
| BALI_ | 105 | GGTCCACGCTCACCTCAGTA | 2a |
| 101 | | GATCGGAAGAGCACACGTCT | |
| BALI_ | 106 | AGACGTGTGCTCTTCCGATC | 2b |
| 102 | | TCGACGATCTAGC | |
| BALI_ | 107 | GGTCCACGCTAGATCGTCGAGA | 2b |
| 103 | | TCGGAAGAGCACACGTCT | |
| BALI_ | 108 | AGACGTGTGCTCTTCCGATCT | |
| 104 | | | |
| BALI_ | 109 | AATGATACGGCGACCACCGAGA | |
| 105 | | TCTACACTCTTTCCCTACACGA | |
| | | CGCTCTTCCGATCTNNNNNNGC | |
| | | GGCTAGCTCTCGACAT | |

(“cage” refers to the Photocleavable spacer modification as shown in FIG. 3, “acrylate” to a 5′ acrydite group, “cy3” refers to a cyanine 3 fluorescent group bound to the 5′ of the molecule, “cy5” refers to a cyanine 5 fluorescent group bound to the 5′ end of a molecule, ‘N’ refers to any nucleotide of A, T, G, or C)

This experiment is designed to measure whether the amount of detection probes bound to a spatial region of a sample (in this case a functionalised hydrogel), and spatially indexed by our technology, can be measured by sequencing. Detection probes are homogeneously distributed on a functionalised coverslip, and two areas (a large one and a small one) are functionalised using different 2-bit spatial barcodes (two indexes each). Sequencing is then used to validate that the barcode assigned to the “large” area is more abundant than the barcode assigned to the “small” area.

An oligo-functionalised hydrogel was prepared by first pre-annealing oligos BALI_92 and BALI_93 by combining them to a final concentration of 15 uM in 2×SSC, heating to 95 C for 2 minutes and cooling down to room temperature for 30 minutes, and then diluting the annealed oligos to a final concentration of 1 uM in degassed gel buffer (4% 19:1 acrylamide:bisacrylamide, 0.3 M NaCl, 60 mM Tris-HCl pH 8). A 80 ul drop of the gel solution was used to coat coverslips functionalised in BIND-Silane (GE healthcare) by incubation for 1 h at room temperature. BALI_92 and BALI_93 are designed to mimic a detection probe with an annealed stabiliser region.

The functionalised gel was first washed 3 times for 5 minutes at in 2×SSC (room temperature). A first ligation was then performed to attach a caged “bridge” molecule to the detection probes. Oligos BALI_94 and BALI_95 were annealed by combining them to a final concentration of 5 uM in 2×SSC, heating to 95 C for 2 minutes and cooling down to room temperature for 30 minutes, and then further diluted to a final concentration of 500 nM in a ligation mix including 1× Quick ligation buffer (NEB) and 100 U/ul T4 DNA ligase. The functionalised coverslips were incubated with the ligation mix for 30 minutes at room temperature, and washed 3 times for 3 minutes at room temperature in 2×SSC.

Following the ligation, a dephosphorylation reaction was performed to remove any phosphate group produced by spontaneous a specific uncaging of the photocage group. This was done by incubating the samples for 30 minutes at 37 C in a mixture including 1× Outsmart buffer (NEB) and 0.05 U/ul shrimp alkaline phosphatase, followed by three washes at room temperature for 5 minutes in 2×SSC.

Uncaging of the first “large” area was then performed on a leica SP5 confocal microscope equipped with a 30 mW 405 nm laser, using a 10× objective and 100% laser power. Uncaging was done for 5 minutes on 20 fields of view (approx 1 mm² each). Following this, the first bit of the spatial barcode was ligated to this area by incubating the sample for 30 minutes at room temperature in a ligation mix including 1× Quick ligation buffer (NEB), 100 U/ul T4 DNA ligase and 500 nM of oligos BALI_96 and BALI_97 annealed as described above. Ligation was followed by 3 washes at room temperature for 5 minutes is 2×SSC.

A second “small” area was then uncaged (as above, 4 fields of view), followed by ligation using annealed oligos BALI_98 ad BALI_99 and by another round of washes.

The first “large” area was then localized again on the microscope using the loss of cy5 fluorescence and the acquisition of cy3 fluorescence as guide, and uncaged again with the same parameters, followed by ligation with oligos BALI_100 and BALI_101. The same was done for the “small” area, with oligos BALI_102 and BALI_103. In between ligation/uncaging steps the sample was washed three times at room temperature for 5 minutes in 2×SSC.

After completion of the spatial barcoding, the signal from the barcoded detection probes was amplified by in-situ RNA transcription by incubating the sample in a transcription mixture containing 130 ul ultrapure H2O, 72 ul NTP mix (from the NEB Hiscribe T7 quick kit) and 14.4 ul of T7 RNA polymerase. Transcription was performed for 2 h at 37 C, after which the gel and transcription mixture were collected, diluted with 130 ul ultrapure H2O, and purified via ethanol precipitation in presence of 0.3 M Sodium acetate.

The recovered RNA was reverse transcribed using the superscript III kit (thermo scientific) according to standard protocols, using BALI_104 as a gene-specific primer. The resulting cDNA was then converted in an Illumina sequencing library using primers BALI_105 and the standard reverse indexed Truseq LT primer (index 006)

The libraries were sequenced using an Illumina MiSeq sequencer (paired end 150 reads) and analysed through a custom bioinformatics pipeline to quantify the abundance of each spatial index combination. Results are shown in FIG. 14.

### Example 11

Increased Signal to Noise Ratio by Using Detection Probes Against Pre-Amplified Transcripts.

TABLE 13

| | SEQ | |
|---|---|---|
| | ID | |
| Name | NO: | Sequence |
| BALI_106 | 110 | [Phosphate]ACATTAGATTGCC |
| | | TGATCGCTGTCGGATTATTACTAT |
| | | CGCATACACTAAAGATA |
| BALI_107 | 111 | CAACGCTGTCAGCTAAGCGCTAAT |
| | | GTTATCTT |
| BALI_108 | 112 | [Phosphate]GTGACGT |
| | | ATGTCGAGAGCTAGC |
| | | CCGTGGACATCT |
| | | NNNNNNNNNNNNNNN |
| | | AGATCGGAAGAGCGTCGTGTAG |
| | | CTCCCTATAGTGAGTCGTATTA |
| | | CTAGCTAGCG |
| | | GATTGCCTGATCGCTGTCG |
| BALI_109 | 113 | [Atto 565]GATTGCCTGATCGCTGTCG |

(‘N’ refers to any nucleotide of A, T, G, or C, “Atto565” refers to the atto565 red fluorescent group bound to the 5′ end of a molecule)

In this experiment we demonstrate the possibility of targeting detection probes against amplified molecules which are produced on top of target RNA transcripts. Specifically, we are producing a DNA concatemer by rolling circle amplification (RCA) following the circularization of a detection probe for an artificial barcode expressed in the genome of a cell population. The circularization is performed through splint ligation, using a second probe (targeted just downstream of the first one on the same expressed barcode) as stabiliser. This signal amplification protocol is known in the art and described in a technique called “starMAP” (see reference at Pubmed ID 29930089).

The detection probe, in this experiment, is targeted to a unique sequence found on the DNA concatemer produced by the amplification. The amplification technique can be used to increase the signal from each target of a detection probe, resulting in increased signal-to-noise ratio for detection.

In this experiment, we detect the DNA concatamers both by direct hybridization with a fluorescent probe (BALI_109), and then by hybridization of a detection probe followed by ligation of a caged bridge molecule, showing that the same pattern of binding is obtained.

Cells expressing an artificial DNA barcode (4t1_barcode cells, provided by a collaborator in our laboratory) were cultured on #1.5 thickness glass coverslips functionalised first with BIND-silane (GE Healthcare), and then overnight with 0.01% poly-L-lysine in complete culture medium (DMEM, 10% fetal bovine serum). Two samples were prepared, one for direct detection by fluorescence in-situ hybridization and one for detection via detection probe hybridization and ligation.

Prior to the experiment, cells were fixed in 4% paraformaldehyde for 10 minutes, washed in PBS, and permeabilized by incubation in Methanol for 10 minutes at −20 C.

After permeabilization, the samples were washed once at room temperature for 5 minutes in PBS supplemented with 0.1% Tween 20 and 0.1 U/ul superase RNAse inhibitor (Thermo Scientific) (from now: PBSTR) and once at room temperature for 5 minutes in hybridization buffer (2×SSC, 10% formamide, 1% Tween 20, 20 mM vanadyl ribonuclease complex, 0.1 mg/ml salmon sperm DNA). The two hybridization probes (BALI_106 and BALI_107) were diluted to 25 uM in ultrapure H2O, heat up at 95 C for 2 minutes, and cooled down to room temperature for 30 minutes and then further diluted to a 100 nM final concentration in hybridization buffer. Hybridization was performed at 40 C overnight.

The following day, the samples were washed twice in PBSTR for 20 minutes each at 37 C, and once in a 1:1 solution of 4×SSC/PBSTR for 20 minutes at 37 C. A ligation mix was then added, including 40 U/ul T4 DNA ligase, 0.1 U/ul Superase RNAse inhibitor, 1×T4 ligase buffer (NEB), and 0.2 mg/ml BSA. The ligation was carried out for 2 h at room temperature, and the samples were then washed twice at room temperature for 5 minutes in PBSTR. Signal amplification was then performed by incubating the samples 2 h at 30 C in an amplification mix including 0.2 U/ul Phi29 DNA polymerase, 250 uM dNTP, 20 uM aminoallyl dUTP, 0.1 U/ul Superase RNAse inhibitor, and 1×Phi29 polymerase buffer (NEB). Finally, the sample was washed twice at room temperature for 5 minutes in PBSTR, and once at room temperature for 5 minutes in PBS.

The amplicons produced in the sample were functionalised with acrylic acid by incubating the samples in 20 mM Acrylic Acid NHS ester in PBS for 2 h at room temperature, followed by two washed at room temperature for 5 minutes in PBS. A thin hydrogel was cast over the cells by coating the coverslips with a 80 ul drop of degassed hydrogel buffer (4% 19:1 acrylamide:bis-acrylamide mix, 2×SSC, 0.05% TEMED, 0.05% Ammonium persulfate) and incubating for 1 h at room temperature. The samples were then digested in digestion buffer (1% SDS, 2×SSC, 0.2 mg/ml NEB Proteinase K enzyme) for 1 h at 37 C in a humidified chamber, and washed 3 times at room temperature for 5 minutes in PBS.

For direct FISH detection, the amplicons were detected by incubating the sample for 30 minutes in presence of a 500 nM dilution of the detection probe (BALI_109) in 2×SSC/10% Formamide, followed by three washes at room temperature for 5 minutes in 2×SSC. Images were acquired on a Leica SP5 confocal microscope.

For detection probe binding and ligation, the samples were incubated for 5 minutes at room temperature in encoding hybridization buffer (2×SSC, 30% formamide), and hybridized with the BALI_108 probe diluted to a final concentration of 225 nM in a encoding hybridization mix including 2×SSC buffer, 30% formamide, 10% dextran sulphate, 1 mg/ml yeast tRNA, and 1:100 NEB murine ribonuclease inhibitor. The samples were then washed twice at 47 C for 30 minutes in encoding hybridization buffer, and once at room temperature for 5 minutes in 2×SSC. A ligation was then performed to attach the caged and fluorescent “bridge” molecule to the detection probes. Oligos BALI_94 and BALI_95 were annealed by combining them to a final concentration of 5 uM in 2×SSC, heating to 95 C for 2 minutes and cooling down to room temperature for 30 minutes, and then further diluted to a final concentration of 500 nM in a ligation mix including 1× Quick ligation buffer (NEB) and 100 U/ul T4 DNA ligase. The functionalised coverslips were incubated with the ligation mix for 30 minutes at room temperature, and washed 3 times for 3 minutes at room temperature in 2×SSC. The samples were then imaged to detect the caged detection probe on the same microscope described above. For both imaging experiments, counter-staining of nuclei was performed in SYTO 16 at 0.33 uM concentration for 10 minutes in 2×SSC. Results are shown in FIG. 15.

1. A method of spatially barcoding one or more locations of a substrate, comprising:
(a) Binding one or more root nucleic acid molecules to the or each location on which the spatial barcode will be constructed, wherein the or each root molecule may comprise a photocleavable group;
(b) Optionally, if the or each root molecule does not comprise a photocleavable group, adding a photocleavable group to the or each root molecule;
(c) Illuminating a location of interest on the substrate to be spatially barcoded, wherein the illumination cleaves or alters the photocleavable group of the or each root molecule present within the location;
(d) Adding an index sequence to the or each root molecule within the location illuminated in step (b), wherein the index sequence comprises a photocleavable group;
(e) Repeating steps (c) and (d) until the desired index sequences are added to form a spatial barcode attached to the or each root molecule within the location.

2. The method according to claim 1, wherein the substrate is inert or living, preferably the substrate is living, preferably the substrate is a tissue.

3. A method of spatially barcoding one or more detection probes, comprising:
(a) Providing a tissue with one or more detection probes bound to one or more biological molecules of interest, wherein the or each detection probe may comprise a photocleavable group;
(b) Optionally, if the or each detection probe does not comprise a photocleavable group, adding a photocleavable group to the or each detection probe;
(c) Illuminating an area of interest within the tissue to be spatially barcoded, wherein the illumination cleaves or alters the photocleavable group of the or each detection probe within the area;
(d) Adding an index sequence of the spatial barcode to the or each detection probe within the area illuminated in step (b), wherein the index sequence comprises a photocleavable group;
(e) Repeating steps (c) and (d) until the desired index sequences are added to form a spatial barcode attached to the or each detection probe within the area of interest.

4. The method according to claim 3, wherein the one or more biological molecules are selected from: nucleic acids, proteins, post-translational protein modifications, metabolites, small bioactive molecules, nucleotides, and drugs.

5. The method according to claim 3 or 4, wherein the one or more detection probes comprise a binding region to bind to a biological molecule, preferably the binding region may be an aptamer, nucleic acid, nucleic acid mimic, protein, or a mixture thereof.

6. A method of analysing one or more transcripts in a tissue, comprising:
(a) Contacting the tissue with one or more detection probes to allow the or each detection probe to bind to a transcript of interest, wherein the or each detection probe comprises a photocleavable group;
(b) Optionally, if the or each detection probe does not comprise a photocleavable group, adding a photocleavable group to the or each detection probe;
(c) Illuminating an area of interest within the tissue to be spatially barcoded, wherein the illumination cleaves or alters the photocleavable group of the or each detection probe within the area;
(d) Adding an index sequence of the spatial barcode to the or each detection probe within the area illuminated in step (b), wherein the index sequence comprises a photocleavable group;
(e) Repeating steps (c) and (d) until the desired index sequences are added to form a spatial barcode attached to the or each detection probe within the area of interest;
(f) Sequencing the one or more spatially barcoded detection probes of step (d) or a derivative thereof.

7. The method according to claim 6, wherein the transcript is RNA, preferably mRNA.

8. The method according to claim 6 or 7, wherein the or each detection probe binds to the polyA region of a transcript of interest.

9. The method according to claim 8, wherein the method further comprises a step of elongating the or each detection probe, preferably at the 3′ end, preferably by reverse transcription.

10. The method according to claim 9, wherein the step of elongating takes places between steps (a) and (b).

11. The method according to any of claims 6-10, wherein the or each detection probe comprises a binding region, wherein the binding region is a nucleic acid, or a nucleic acid mimic.

12. A method of analysing one or more markers within a tissue, comprising:
(a) Contacting the tissue with one or more detection probes to allow the or each detection probe to bind to a marker of interest, wherein the or each detection probe comprises a photocleavable group;
(b) Optionally, if the or each detection probe does not comprise a photocleavable group, adding a photocleavable group to the or each detection probe;
(c) Illuminating an area of interest within the tissue to be spatially barcoded, wherein the illumination cleaves or alters the photocleavable group of the or each detection probe within the area;
(d) Adding an index sequence of the spatial barcode to the or each detection probe within the area illuminated in step (b), wherein the index sequence comprises a photocleavable group;
(e) Repeating steps (c) and (d) until the desired index sequences are added to form a spatial barcode attached to the or each detection probe within the area of interest;
(f) Sequencing the one or more spatially barcoded detection probes of step (d) or a derivative thereof.

13. The method according to claim 12, wherein the or each marker is a biological molecule, preferably selected from: proteins, post-translational protein modifications, metabolites, small bioactive molecules, nucleotides, or drugs.

14. The method according to claim 13, wherein the or each marker is a protein, and the method is a method of analysing one or more proteins in the tissue.

15. The method according to claims 12-14, wherein the or each detection probe comprises binding region, preferably wherein the binding region is a protein, aptamer, nucleic acid, nucleic acid mimic or a mixture thereof, preferably wherein the binding region is an antibody or a nanobody.

16. A method of analysing one or more transcripts and one or more markers in a tissue, comprising:
(a) Contacting the tissue with a plurality of detection probes to allow the detection probes to bind to both a nucleic acid and a marker of interest in the tissue, wherein the or each detection probe comprises a photocleavable group;
(b) Optionally, if the or each detection probe does not comprise a photocleavable group, adding a photocleavable group to the or each detection probe;
(c) Illuminating an area of interest within the tissue to be spatially barcoded, wherein the illumination cleaves or alters the photocleavable group of the or each detection probe within the area;
(d) Adding an index sequence of the spatial barcode to the or each detection probe within the area illuminated in step (b), wherein the index sequence comprises a photocleavable group;
(e) Repeating steps (c) and (d) until the desired index sequences are added to form a spatial barcode attached to the or each detection probe within the area of interest;
(f) Sequencing the one or more spatially barcoded detection probes of step (d) or derivatives thereof.

17. The method according to claim 16, wherein the one or more markers are selected from: proteins, post-translational protein modifications, metabolites, small bioactive molecules, nucleotides, or drugs, preferably the one or more markers are proteins.

18. The method according to claim 16 or 17, wherein the plurality of detection probes comprises: one or more detection probes comprising a binding region which is a nucleic acid, nucleic acid mimic, or aptamer, and one or more detection probes comprising a binding region which is a protein, preferably the protein binding region is an antibody or a nanobody.

19. The method according to any of claims 1-18, wherein the method further comprises a step of assigning a unique spatial barcode to each location or area of interest before step (c).

20. The method according to any of claims 1-19, wherein the location or area of interest may be a two-dimensional or three-dimensional region, preferably a three-dimensional region.

21. The method according to claim 20, wherein the three-dimensional region is between 1 μm³-150 mm³ in size, between 1 μm³-1 mm³ in size, between 1 μm³-1,000,000 μm³ in size, between 1 μm³-200,000 μm³ in size, between 1 μm³-20,000 μm³ in size, or between 1 μm³- 1000 μm³ in size.

22. The method according to any preceding claim, wherein the area or location of interest comprises a collection of cells, preferably from 1 up to 100,000,000 cells, 1,000,000 cells, 1000 cells, 100 cells, 10 cells, preferably the area or location of interest comprises a single cell or a sub-cellular region or compartment.

23. The method according to any preceding claim, wherein the method further comprises a step of selecting one or more locations or areas of interest, preferably multiple locations or areas of interest are selected, preferably prior to step (a).

24. The method according to any of claim 3-11, or 16-23, wherein the biological molecule is a nucleic acid, and wherein the method further comprises a step of pre-amplification, preferably pre-amplification of the nucleic acids of interest or the transcripts of interest, preferably prior to step (a).

25. The method according to any of claim 3-11, or 16-24, wherein the biological molecule is a nucleic acid, and wherein step (a) of the method may comprise contacting the tissue with one or more, or a plurality of, split detection probes to allow the or each split detection probe to bind to a nucleic acid of interest and form a whole detection probe, preferably wherein contacting the tissue with the split detection probes comprises contacting the tissue with first and second parts of each detection probe.

26. The method according to any preceding claim, wherein the or each detection probe is as defined in claim 36.

27. The method according to any preceding claim, wherein the or each index sequence is selected from the library of index sequences as defined in claim 42.

28. The method according to any preceding claim, wherein step (b) comprises the addition of a bridge molecule to the or each root molecule or detection probe, wherein the bridge molecule is between 5 to 40 nucleotides in length and comprises a photocleavable group at the 5′ end or 3′end.

29. The method according to any preceding claim, wherein the photocleavable group is a light-sensitive group which protects the 5′ or 3′ end, preferably the photocleavable group comprises a cage, preferably the photocleavable group comprises a nitrobenzyl group, dimethoxy-nitrobenzyl group, nitrophenyl group, or nitroveratryl group.

30. The method according to any preceding claim, wherein the photocleavable group is cleaved or altered by illumination, preferably illumination cleaves or alters the photocleavable groups in the illuminated location or area.

31. The method according to claim 30, wherein the or each location or area of interest is illuminated by light having a wavelength between 300-600 nm, between 310 nm-570 nm, between 320 nm-550 nm, between 330 nm-520 nm, between 340 nm-480 nm, between 350 nm-450 nm, or between 360 nm-420 nm, preferably in a one-photon photorelease process.

32. The method according to claim 30, wherein the or each location or area of interest is illuminated by light having a wavelength between 680 nm and 900 nm, between 700 and 850 nm, or between 720 and 800 nm, preferably in a two-photon photorelease process.

33. The method according to any preceding claim, wherein the or each index sequence is added by ligation, preferably by ligation onto the 5′ or 3′ end of a root molecule, bridge molecule, or detection probe present in the location or area illuminated in step (c).

34. The method according to claim 33, wherein the ligation is by a ligase enzyme, preferably a ligase selected from T4 ligase, T3 ligase, or Taq ligase.

35. A tissue produced by the method of any of claims 3-34, wherein the tissue comprises spatially barcoded detection probes.

36. A detection probe comprising:
(i) A binding region;
(ii) A species barcode; and
(iii) A photocleavable group

37. The detection probe according to claim 36, wherein the binding region allows the detection probe to bind to a biological molecule, preferably the binding region comprises a nucleic acid, nucleic acid mimic, aptamer, or a protein.

38. The detection probe according to claim 36 or 37, further comprising an amplification region, preferably wherein the amplification region comprises a promoter for a polymerase, preferably the amplification region is a nucleic acid.

39. The detection probe according to claims 36-38 wherein the species barcode allows identification of the biological molecule that the detection probe binds to, preferably the species barcode is a nucleic acid.

40. The detection probe according to claims 36-39, further comprising a unique molecule identifier (UMI), preferably wherein the UMI allows quantification of detection probes, preferably wherein the UMI is unique to the detection probe, preferably wherein the UMI is a nucleic acid.

41. The detection probe according to claims 36-40, wherein the photocleavable group is a light-sensitive group which protects the 5′ or 3′ end of a the detection probe, preferably the photocleavable group comprises a cage, preferably the photocleavable group comprises a nitrobenzyl group, dimethoxy-nitrobenzyl group, nitrophenyl group, or nitroveratryl group.

42. A library of index sequences, wherein each index sequence comprises:
(i) A total length of between 5 and 50 nucleotides; and
(ii) A photocleavable group bound to one or both of the 5′ or 3′ ends.

43. The library according to claim 42, wherein each index sequence is a nucleic acid.

44. The library according to claim 42 or 43 wherein each index sequence comprises an overhang of preferably 4-15 nucleotides in length at the 5′ and 3′ end, preferably 6 or 7 nucleotides in length at the 5′ and 3′ end.

45. The library according to claims 42-44, wherein each index sequence has a total length of between 19-20 nucleotides.

46. A spatial barcode comprising a plurality of index sequences, wherein the index sequences are selected from the library according to any of claims 43-45.

47. The spatial barcode according to claim 46, wherein the spatial barcode comprises between 1 to 50 index sequences.

48. A spatial barcode according to claim 46 or 47, wherein the spatial barcode is between 10 and 250 nucleotides in length.

49. A spatially barcoded detection probe comprising a detection probe linked to a spatial barcode, wherein the spatial barcode is as defined in any of claims 46-48.

50. The spatially barcoded detection probe according to claim 49, wherein the detection probe is as defined in any of claims 36-41.

51. A kit, the kit comprising:
a library of index sequences as defined in any of claims 42-45, one or more detection probes as defined in any of claims 36-41, optionally a ligase enzyme, and optionally one or more reagents.

52. A system for spatial barcoding, the system comprising:
(i) an instrument for viewing a substrate;
(ii) a light source for illuminating one or more locations of the substrate;
(iii) microfluidic circuit for delivering one or more index sequences and reagents to the substrate; and
(iv) a processor for implementing software operable to control the instrument, light source, and microfluidic circuit.

53. The system according to claim 52, wherein the substrate is a tissue.

54. The system according to claim 52 or 53, wherein the system is for spatially barcoding one or more locations, detection probes and/or markers.

55. The system according to any of claims 52-54, wherein the one or more locations are areas.

56. The system according to any of claims 52-55 wherein the instrument is further for directing the light source, preferably the instrument is a microscope, preferably a light microscope.

57. The system according to any of claims 52-56 further comprising an optical system, wherein the optical system comprises an element to direct illumination to the or each location or area of interest, preferably the element is a movable mirror, preferably the optical system is comprised within a microscope.

58. The system according to any of claims 52-57, wherein the processor implements software which is operable to:
(i) conduct image processing of the tissue;
(ii) assign a spatial barcode to each selected location or area of interest of the substrate or tissue;
(iii) control illumination of the selected locations or areas of interest; and/or
(iv) control fluid flow through the microfluidic circuit.

59. The system according to any of claims 52-58, wherein the microfluidic circuit comprises one or more channels for delivering one or more index sequences and reagents to the substrate, preferably wherein the channels are in fluid communication with the substrate.

60. The system according to any of claims 52-59, wherein the microfluidic circuit comprises one or more storage chambers for storing the index sequences and reagents, preferably wherein the one or more storage chambers are in fluid communication with the channels and the substrate.


SEQUENCE LISTING (113 sequences)

SEQ ID NO: 1 - DNA, 80 bp, Artificial Sequence
  BALI_01 oligonucleotide
  misc_feature (1)..(1): [Cy3]  [cage]
ctaggtcgatgtcgagagcaattagagtcgcgccttaagatacagatcggaagagcgtcgtgtagcgcgccttaagatac

SEQ ID NO: 2 - DNA, 35 bp, Artificial Sequence
  BALI_02 oligonucleotide
gtatcttaaggcgcgactctaattgctctcgacat

SEQ ID NO: 3 - DNA, 18 bp, Artificial Sequence
  BALI_03 oligonucleotide
ggtagtatagctaccatg

SEQ ID NO: 4 - DNA, 18 bp, Artificial Sequence
  BALI_04 oligonucleotide
cgacctagcatggtagct

SEQ ID NO: 5 - DNA, 80 bp, Artificial Sequence; identical to SEQ ID NO: 1
  BALI_05 oligonucleotide
  misc_feature (1)..(1): [Cy3]  [cage]
  misc_feature (80)..(80): [Aminolink C6]

SEQ ID NO: 6 - DNA, 35 bp, Artificial Sequence; identical to SEQ ID NO: 2
  BALI_06 oligonucleotide

SEQ ID NO: 7 - DNA, 18 bp, Artificial Sequence
  BALI_07 oligonucleotide
  misc_feature (1)..(1): [Cy5]
ggtagtatagctaccatg

SEQ ID NO: 8 - DNA, 18 bp, Artificial Sequence
  BALI_08 oligonucleotide
cgacctagcatggtagct

SEQ ID NO: 9 - DNA, 102 bp, Artificial Sequence
  BALI_09 oligonucleotide
  misc_feature (102)..(102): [Aminolink C6]
ggtagtatatgtcgagagctagccgcgccttaagataccaattagagtagatcggaagagcgtcgtgtagctccctatagtgagtcgtattactagctagcg

SEQ ID NO: 10 - DNA, 15 bp, Artificial Sequence
  BALI_10 oligonucleotide
gctagctctcgacat

SEQ ID NO: 11 - DNA, 19 bp, Artificial Sequence
  BALI_11 oligonucleotide
  misc_feature (1)..(1): [Atto488] [cage]
gtacctgtcagctaccatg

SEQ ID NO: 12 - DNA, 19 bp, Artificial Sequence
  BALI_12 primer
  misc_feature (1)..(1): phosphate
atactacccatggtagctg

SEQ ID NO: 13 - DNA, 18 bp, Artificial Sequence
  BALI_13 oligonucleotide
  misc_feature (1)..(1): [Cy5] [cage]
gtatcgagctctatacac

SEQ ID NO: 14 - DNA, 18 bp, Artificial Sequence
  BALI_14 oligonucleotide
  misc_feature (1)..(1): phosphate
acaggtacgtgtatagag

SEQ ID NO: 15 - DNA, 19 bp, Artificial Sequence
  BALI_15 oligonucleotide
  misc_feature (1)..(1): [Atto565] [cage]
gtgagcgtcggacacctac

SEQ ID NO: 16 - DNA, 19 bp, Artificial Sequence
  BALI_16 oligonucleotide
  misc_feature (1)..(1): phosphate
ctcgatacgtaggtgtccg

SEQ ID NO: 17 - DNA, 18 bp, Artificial Sequence
  BALI_017 oligonucleotide
  misc_feature (1)..(1): phosphate
gtgcgtgcaccacagtcg

SEQ ID NO: 18 - DNA, 18 bp, Artificial Sequence
  BALI_018 oligonucleotide
  misc_feature (1)..(1): phosphate
gtgaccgcaccacagtcg

SEQ ID NO: 19 - DNA, 18 bp, Artificial Sequence
  BALI_019 oligonucleotide
acgcaccgggactcgtgc

SEQ ID NO: 20 - DNA, 18 bp, Artificial Sequence
  BALI_020 oligonucleotide
ggtcaccgggactcgtgc

SEQ ID NO: 21 - DNA, 19 bp, Artificial Sequence
  BALI_021 oligonucleotide
  misc_feature (1)..(1): phosphate
gtgacgtgcaccacagtcg

SEQ ID NO: 22 - DNA, 19 bp, Artificial Sequence
  BALI_022 oligonucleotide
  misc_feature (1)..(1): phosphate
gtggaccgcaccacagtcg

SEQ ID NO: 23 - DNA, 19 bp, Artificial Sequence
  BALI_023 oligonucleotide
acgtcaccgggactcgtgc

SEQ ID NO: 24 - DNA, 19 bp, Artificial Sequence
  BALI_024 oligonucleotide
ggtccaccgggactcgtgc

SEQ ID NO: 25 - DNA, 12 bp, Artificial Sequence
  BALI_025 oligonucleotide
gcacgagtcccg

SEQ ID NO: 26 - DNA, 100 bp, Artificial Sequence
  BALI_26 oligonucleotide
  misc_feature (1)..(1): phosphate
  misc_feature (100)..(100): [aminolink C6]
gtgcgtatgtcgagagctagccgcgccttaagataccaattagagtagatcggaagagcgtcgtgtagctccctatagtgagtcgtattactagctagcg

SEQ ID NO: 27 - DNA, 18 bp, Artificial Sequence
  BALI_27 oligonucleotide
acgcaccgggactcgtgc

SEQ ID NO: 28 - DNA, 19 bp, Artificial Sequence
  BALI_28 oligonucleotide
  misc_feature (1)..(1): phosphate
gtggaccgcacgagtcccg

SEQ ID NO: 29 - DNA, 19 bp, Artificial Sequence
  BALI_29 oligonucleotide
ggtccaccgactgtggtgc

SEQ ID NO: 30 - DNA, 18 bp, Artificial Sequence
  BALI_30 oligonucleotide
  misc_feature (1)..(1): phosphate
gtgcgtgcaccacagtcg

SEQ ID NO: 31 - DNA, 22 bp, Artificial Sequence
  T7 promoter
taatacgactcactatagggag

SEQ ID NO: 32 - DNA, 77 bp, Artificial Sequence
  BALI_31 oligonucleotide
  misc_feature (1)..(1): [NH2]
ctactacggctagcctaatacgactcactatagggagactctaattggtatagaattccgcggctagctctcgacat

SEQ ID NO: 33 - DNA, 71 bp, Artificial Sequence
  BALI_32 oligonucleotide
  misc_feature (1)..(1): phosphate
  misc_feature (71)..(71): [cy5]
gtgcgtatgtcgagagctagccgcggaattctataccaattagagtctccctatagtgagtcgtattaggc

SEQ ID NO: 34 - DNA, 18 bp, Artificial Sequence
  BALI_33 oligonucleotide
acgcaccgggactcgtgc

SEQ ID NO: 35 - DNA, 19 bp, Artificial Sequence
  BALI_34 oligonucleotide
  misc_feature (1)..(1): phosphate
gtggaccgcacgagtcccg

SEQ ID NO: 36 - DNA, 18 bp, Artificial Sequence
  BALI_35 oligonucleotide
  misc_feature (1)..(1): phosphate
gtgcgtgcacgtatggcg

SEQ ID NO: 37 - DNA, 19 bp, Artificial Sequence
  BALI_36 oligonucleotide
ggtccaccgccatacgtgc

SEQ ID NO: 38 - DNA, 18 bp, Artificial Sequence
  BALI_37 oligonucleotide
  misc_feature (1)..(1): phosphate
gtgcgtgcaccacagtcg

SEQ ID NO: 39 - DNA, 19 bp, Artificial Sequence
  BALI_38 oligonucleotide
ggtccaccgactgtggtgc

SEQ ID NO: 40 - DNA, 18 bp, Artificial Sequence
  BALI_39 oligonucleotide
  misc_feature (1)..(1): phosphate
gtgcgtgcaccgacctcg

SEQ ID NO: 41 - DNA, 19 bp, Artificial Sequence
  BALI_40 oligonucleotide
ggtccaccgaggtcggtgc

SEQ ID NO: 42 - DNA, 18 bp, Artificial Sequence
  BALI_41 oligonucleotide
  misc_feature (1)..(1): phosphate
gtgcgtgcaccgtgtacg

SEQ ID NO: 43 - DNA, 19 bp, Artificial Sequence
  BALI_42 oligonucleotide
ggtccaccgtacacggtgc

SEQ ID NO: 44 - DNA, 18 bp, Artificial Sequence
  BALI_43 oligonucleotide
  misc_feature (1)..(1): phosphate
gtgcgtgcacctagatcg

SEQ ID NO: 45 - DNA, 19 bp, Artificial Sequence
  BALI_44 oligonucleotide
ggtccaccgatctaggtgc

SEQ ID NO: 46 - DNA, 18 bp, Artificial Sequence
  BALI_45 oligonucleotide
  misc_feature (1)..(1): phosphate
gtgcgtgcacgagtcccg

SEQ ID NO: 47 - DNA, 19 bp, Artificial Sequence
  BALI_46 oligonucleotide
ggtccaccgggactcgtgc

SEQ ID NO: 48 - DNA, 18 bp, Artificial Sequence
  BALI_47 oligonucleotide
  misc_feature (1)..(1): phosphate
gtgcgtgcacggacgacg

SEQ ID NO: 49 - DNA, 19 bp, Artificial Sequence
  BALI_48 oligonucleotide
ggtccaccgtcgtccgtgc

SEQ ID NO: 50 - DNA, 18 bp, Artificial Sequence
  BALI_49 oligonucleotide
  misc_feature (1)..(1): phosphate
gtgcgtgcacgtatggcg

SEQ ID NO: 51 - DNA, 19 bp, Artificial Sequence
  BALI_50 oligonucleotide
ggtccaccgccatacgtgc

SEQ ID NO: 52 - DNA, 18 bp, Artificial Sequence
  BALI_51 oligonucleotide
  misc_feature (1)..(1): phosphate
gtgcgtgcactatctgcg

SEQ ID NO: 53 - DNA, 19 bp, Artificial Sequence
  BALI_52 oligonucleotide
ggtccaccgcagatagtgc

SEQ ID NO: 54 - DNA, 18 bp, Artificial Sequence
  BALI_53 oligonucleotide
  misc_feature (1)..(1): phosphate
gtgcgtgcactcaggtcg

SEQ ID NO: 55 - DNA, 19 bp, Artificial Sequence
  BALI_54 oligonucleotide
ggtccaccgacctgagtgc

SEQ ID NO: 56 - DNA, 18 bp, Artificial Sequence
  BALI_55 oligonucleotide
  misc_feature (1)..(1): phosphate
gtgcgtgcagagatgacg

SEQ ID NO: 57 - DNA, 19 bp, Artificial Sequence
  BALI_56 oligonucleotide
ggtccaccgtcatctctgc

SEQ ID NO: 58 - DNA, 18 bp, Artificial Sequence
  BALI_57 oligonucleotide
  misc_feature (1)..(1): phosphate
gtgcgtgccacacatccg

SEQ ID NO: 59 - DNA, 19 bp, Artificial Sequence
  BALI_58 oligonucleotide
ggtccaccggatgtgtggc

SEQ ID NO: 60 - DNA, 18 bp, Artificial Sequence
  BALI_59 oligonucleotide
  misc_feature (1)..(1): phosphate
gtgcgtgccacatgctcg

SEQ ID NO: 61 - DNA, 19 bp, Artificial Sequence
  BALI_60 oligonucleotide
ggtccaccgagcatgtggc

SEQ ID NO: 62 - DNA, 18 bp, Artificial Sequence
  BALI_61 oligonucleotide
  misc_feature (1)..(1): phosphate
gtgcgtgccacggtagcg

SEQ ID NO: 63 - DNA, 19 bp, Artificial Sequence
  BALI_62 oligonucleotide
ggtccaccgctaccgtggc

SEQ ID NO: 64 - DNA, 18 bp, Artificial Sequence
  BALI_63 oligonucleotide
  misc_feature (1)..(1): phosphate
gtgcgtgccagccgatcg

SEQ ID NO: 65 - DNA, 19 bp, Artificial Sequence
  BALI_64 oligonucleotide
ggtccaccgatcggctggc

SEQ ID NO: 66 - DNA, 18 bp, Artificial Sequence
  BALI_65 oligonucleotide
  misc_feature (1)..(1): phosphate
gtgcgtgccagtatagcg

SEQ ID NO: 67 - DNA, 19 bp, Artificial Sequence
  BALI_66 oligonucleotide
ggtccaccgctatactggc

SEQ ID NO: 68 - DNA, 18 bp, Artificial Sequence
  BALI_67 oligonucleotide
  misc_feature (1)..(1): phosphate
gtgcgtgctacaccggcg

SEQ ID NO: 69 - DNA, 19 bp, Artificial Sequence
  BALI_68 oligonucleotide
ggtccaccgccggtgtagc

SEQ ID NO: 70 - DNA, 18 bp, Artificial Sequence
  BALI_69 oligonucleotide
  misc_feature (1)..(1): phosphate
gtgcgtgctactcgtccg

SEQ ID NO: 71 - DNA, 19 bp, Artificial Sequence
  BALI_70 oligonucleotide
ggtccaccggacgagtagc

SEQ ID NO: 72 - DNA, 18 bp, Artificial Sequence
  BALI_71 oligonucleotide
  misc_feature (1)..(1): phosphate
gtgcgtgctagactcccg

SEQ ID NO: 73 - DNA, 19 bp, Artificial Sequence
  BALI_72 oligonucleotide
ggtccaccgggagtctagc

SEQ ID NO: 74 - DNA, 18 bp, Artificial Sequence
  BALI_73 oligonucleotide
  misc_feature (1)..(1): phosphate
gtgcgtgctagctgagcg

SEQ ID NO: 75 - DNA, 19 bp, Artificial Sequence
  BALI_74 oligonucleotide
ggtccaccgctcagctagc

SEQ ID NO: 76 - DNA, 18 bp, Artificial Sequence
  BALI_75 oligonucleotide
  misc_feature (1)..(1): phosphate
gtgcgtgctcactcaccg

SEQ ID NO: 77 - DNA, 19 bp, Artificial Sequence
  BALI_76 oligonucleotide
ggtccaccggtgagtgagc

SEQ ID NO: 78 - DNA, 136 bp, Artificial Sequence
  BALI_77 oligonucleotide
  misc_feature (1)..(1): [CAGE]
  misc_feature (22)..(36): N is any nucleotide
gtgaccatgtcgagagctagcnnnnnnnnnnnnnnncatcgtgagtagatcggaagagcgtcgtgtagctccctatagtgagtcgtattactagctagcgttgaagaagatggtgcgctcctggacgtagccttcg

SEQ ID NO: 79 - DNA, 136 bp, Artificial Sequence
  BALI_78 oligonucleotide
  misc_feature (1)..(1): [CAGE]
  misc_feature (22)..(36): N is any nucleotide
gtgaccatgtcgagagctagcnnnnnnnnnnnnnnncatcgtgagtagatcggaagagcgtcgtgtagctccctatagtgagtcgtattactagctagcgcttgaagtcgatgcccttcagctcgatgcggttcac

SEQ ID NO: 80 - DNA, 136 bp, Artificial Sequence
  BALI_79 oligonucleotide
  misc_feature (1)..(1): [CAGE]
  misc_feature (22)..(36): N is any nucleotide
gtgaccatgtcgagagctagcnnnnnnnnnnnnnnncatcgtgagtagatcggaagagcgtcgtgtagctccctatagtgagtcgtattactagctagcgtgcttgtcggccatgatatagacgttgtggctgttg

SEQ ID NO: 81 - DNA, 136 bp, Artificial Sequence
  BALI_80 oligonucleotide
  misc_feature (1)..(1): [CAGE]
  misc_feature (22)..(36): N is any nucleotide
gtgaccatgtcgagagctagcnnnnnnnnnnnnnnncatcgtgagtagatcggaagagcgtcgtgtagctccctatagtgagtcgtattactagctagcgcatgtgatcgcgcttctcgttggggtctttgctcag

SEQ ID NO: 82 - DNA, 136 bp, Artificial Sequence
  BALI_81 oligonucleotide
  misc_feature (1)..(1): [CAGE]
  misc_feature (22)..(36): N is any nucleotide
gtgaccatgtcgagagctagcnnnnnnnnnnnnnnnggtacagatgagatcggaagagcgtcgtgtagctccctatagtgagtcgtattactagctagcggatggccatgttatcctcctcgcccttgctcaccat

SEQ ID NO: 83 - DNA, 136 bp, Artificial Sequence
  BALI_82 oligonucleotide
  misc_feature (1)..(1): [CAGE]
  misc_feature (22)..(36): N is any nucleotide
gtgaccatgtcgagagctagcnnnnnnnnnnnnnnnggtacagatgagatcggaagagcgtcgtgtagctccctatagtgagtcgtattactagctagcgctcggggaaggacagcttcaagtagtcggggatgtc

SEQ ID NO: 84 - DNA, 136 bp, Artificial Sequence
  BALI_83 oligonucleotide
  misc_feature (1)..(1): [CAGE]
  misc_feature (22)..(36): N is any nucleotide
gtgaccatgtcgagagctagcnnnnnnnnnnnnnnnggtacagatgagatcggaagagcgtcgtgtagctccctatagtgagtcgtattactagctagcgcttgtaggtggtcttgacctcagcgtcgtagtggcc

SEQ ID NO: 85 - DNA, 136 bp, Artificial Sequence
  BALI_84 oligonucleotide
  misc_feature (1)..(1): [CAGE]
  misc_feature (22)..(36): N is any nucleotide
gtgaccatgtcgagagctagcnnnnnnnnnnnnnnnggtacagatgagatcggaagagcgtcgtgtagctccctatagtgagtcgtattactagctagcgctgttccacgatggtgtagtcctcgttgtgggaggt

SEQ ID NO: 86 - DNA, 15 bp, Artificial Sequence
  BALI_85 oligonucleotide
gctagctctcgacat

SEQ ID NO: 87 - DNA, 32 bp, Artificial Sequence
  BALI_86 oligonucleotide
agacgtgtgctcttccgatctcagctaccatg

SEQ ID NO: 88 - DNA, 38 bp, Artificial Sequence
  BALI_87 oligonucleotide
ggtcaccatggtagctgagatcggaagagcacacgtct

SEQ ID NO: 89 - DNA, 32 bp, Artificial Sequence
  BALI_88 oligonucleotide
agacgtgtgctcttccgatctgacaatgaggc

SEQ ID NO: 90 - DNA, 38 bp, Artificial Sequence
  BALI_89 oligonucleotide
ggtcacgcctcattgtcagatcggaagagcacacgtct

SEQ ID NO: 91 - DNA, 21 bp, Artificial Sequence
  BALI_90 oligonucleotide
agacgtgtgctcttccgatct

SEQ ID NO: 92 - DNA, 28 bp, Artificial Sequence
  BALI_91 oligonucleotide
taatacgactcactatagggagctacac

SEQ ID NO: 93 - DNA, 58 bp, Artificial Sequence
  Illumina universal FW Truseq
aatgatacggcgaccaccgagatctacactctttccctacacgacgctcttccgatct

SEQ ID NO: 94 - DNA, 63 bp, Artificial Sequence
  Illumina Truseq LT rev. index 006
caagcagaagacggcatacgagatattggcgtgactggagttcagacgtgtgctcttccgatc

SEQ ID NO: 95 - DNA, 63 bp, Artificial Sequence
  Illumina Truseq LT rev. index 012
caagcagaagacggcatacgagattacaaggtgactggagttcagacgtgtgctcttccgatc

SEQ ID NO: 96 - DNA, 90 bp, Artificial Sequence
  BALI_92 oligonucleotide
  misc_feature (1)..(1): [acrylate]
cgacatcggcaagctactacggctagcctaatacgactcactatagggagactctaattggtatagaattccgcggctagctctcgacat

SEQ ID NO: 97 - DNA, 72 bp, Artificial Sequence
  BALI_93 oligonucleotide
  misc_feature (1)..(1): phosphate
gtgacgtatgtcgagagctagccgcggaattctataccaattagagtctccctatagtgagtcgtattaggc

SEQ ID NO: 98 - DNA, 18 bp, Artificial Sequence
  BALI_94 oligonucleotide
  misc_feature (1)..(1): [cy5] [cage]
gagcgtgcaccacagtcg

SEQ ID NO: 99 - DNA, 19 bp, Artificial Sequence
  BALI_95 oligonucleotide
acgtcaccgactgtggtgc

SEQ ID NO: 100 - DNA, 19 bp, Artificial Sequence
  BALI_96 oligonucleotide
  misc_feature (1)..(1): [cy3] [cage]
gtggaccgcacgtatggcg

SEQ ID NO: 101 - DNA, 18 bp, Artificial Sequence
  BALI_97 oligonucleotide
acgctccgccatacgtgc

SEQ ID NO: 102 - DNA, 19 bp, Artificial Sequence
  BALI_98 oligonucleotide
  misc_feature (1)..(1): [cy3] [cage]
gtggaccgcacgagtcccg

SEQ ID NO: 103 - DNA, 18 bp, Artificial Sequence
  BALI_99 oligonucleotide
acgctccgggactcgtgc

SEQ ID NO: 104 - DNA, 33 bp, Artificial Sequence
  BALI_100 oligonucleotide
agacgtgtgctcttccgatctactgaggtgagc

SEQ ID NO: 105 - DNA, 40 bp, Artificial Sequence
  BALI_101 oligonucleotide
ggtccacgctcacctcagtagatcggaagagcacacgtct

SEQ ID NO: 106 - DNA, 33 bp, Artificial Sequence
  BALI_102 oligonucleotide
agacgtgtgctcttccgatctcgacgatctagc

SEQ ID NO: 107 - DNA, 40 bp, Artificial Sequence
  BALI_103 oligonucleotide
ggtccacgctagatcgtcgagatcggaagagcacacgtct

SEQ ID NO: 108 - DNA, 21 bp, Artificial Sequence
  BALI_104 oligonucleotide
agacgtgtgctcttccgatct

SEQ ID NO: 109 - DNA, 82 bp, Artificial Sequence
  BALI_105 oligonucleotide
  misc_feature (59)..(64): N is any nucleotide
aatgatacggcgaccaccgagatctacactctttccctacacgacgctcttccgatctnnnnnngcggctagctctcgacat

SEQ ID NO: 110 - DNA, 54 bp, Artificial Sequence
  BALI_106 oligonucleotide
  misc_feature (1)..(1): phosphate
acattagattgcctgatcgctgtcggattattactatcgcatacactaaagata

SEQ ID NO: 111 - DNA, 32 bp, Artificial Sequence
  BALI_107 oligonucleotide
caacgctgtcagctaagcgctaatgttatctt

SEQ ID NO: 112 - DNA, 122 bp, Artificial Sequence
  BALI_108 oligonucleotide
  misc_feature (1)..(1): phosphate
  misc_feature (35)..(49): N is any nucleotide
gtgacgtatgtcgagagctagcccgtggacatctnnnnnnnnnnnnnnnagatcggaagagcgtcgtgtagctccctatagtgagtcgtattactagctagcggattgcctgatcgctgtcg

SEQ ID NO: 113 - DNA, 19 bp, Artificial Sequence
  BALI_109 oligonucleotide
  misc_feature (1)..(1): [Atto 565]
gattgcctgatcgctgtcg